(19) United States
(12) Patent Application Publication
(10) Pub. No.: US 2009/0264469 A1
(43) Pub. Date: Oct. 22, 2009

(54) NOVEL DICARBOXYLIC ACID DERIVATIVES

(75) Inventors: Marc Capet, Melesse (FR); Nicolas Levoin, Mordelles (FR); Isabelle Berrebi-Bertrand, Pace (FR); Olivia Poupardin, Varois Et Chaignot (FR); Philippe Robert, Pace (FR); Jean-Charles Schwartz, Paris (FR); Jeanne-Marie Lecomte, Paris (FR); Thennati Rajamannar, Baroda (IN); Ranjan Kumar Pal, Baroda (IN); Biswajit Samanta, Baroda (IN); Jignesh K. Jivani, Baroda (IN); Bhavesh M. Panchal, Vallabh Vidyanagar (IN); Isha H. Bhatt, Baroda (IN); Jayraj D. Aradhye, Baroda (IN)

Correspondence Address: VOLPE AND KOENIG, P.C., UNITED PLAZA, SUITE 1600, 30 SOUTH 17TH STREET, PHILADELPHIA, PA, 19103, US

(73) Assignees: BIO-PROJET, Paris, FR (FR); SUN PHARMACEUTICAL INDUSTRIES LTD., Baroda Gujarat, IN (IN)

(21) Appl. No.: 11/577,792

(22) PCT Filed: Oct. 18, 2005
(86) PCT No.: PCT/IB05/03113

§ 371 Date: Jul. 1, 2009

(30) Foreign Application Priority Data
Oct. 22, 2004 (EP) 04292517.2

(51) Int. Cl. A61K 31/196; A61K 31/4535; A61K 31/445; A61K 31/381; A61K 31/216; C07D 409/12; C07D 211/62; C07C 229/48; C07C 229/34; A61P 37/00; A61P 29/00

(52) U.S. Cl. 514/326; 514/330; 514/438; 514/530; 514/563; 514/567; 546/213; 546/225; 560/43; 562/452; 562/456; 562/457

(57) ABSTRACT

The present patent application concerns new compounds of formula (I): displaying agonistic activity at sphingosine-1-phosphate (S1P) receptors, their process of preparation and their use as immunosuppressive agents.

The present patent application concerns new compounds displaying agonistic activity at sphingosine-1-phosphate (S1P) receptors, their process of preparation and their use as immunosuppressive agents.

Among other effects, indirect or direct S1P receptor agonists inhibit thymic egress and lymphocyte recirculation (Rosen et al, Immunol. Rev. 2003, 195, 160).

Inhibition of lymphocyte egress is associated with clinically useful immunosuppression in both transplantation and autoimmune diseases.

Agonism of sphingosine-1-phosphate receptors (particularly S1P1 receptors) induces accelerated homing of lymphocytes to lymph nodes and Peyer's patches without lymphodepletion. Such immunosuppression is desirable to prevent rejection after organ transplantation and in the treatment of autoimmune disorders.

Immunosuppressive agents have been shown to be useful in a wide variety of autoimmune and chronic inflammatory diseases including transplant rejection, multiple sclerosis, lupus erythematosus, rheumatoid arthritis, inflammatory bowel disease, psoriasis, asthma, myocarditis, atopic dermatitis, type-1 diabetes, athero-sclerosis, glomerulonephritis, lymphocytic leukemias, lymphomas, multiorgan failure, sepsis, pneumonia, etc.

This mechanism was recently shown to be operant in the well demonstrated immunosuppression of FTY 720, a drug currently submitted to clinical trials in the prevention of organ transplant rejection (Matioubian et al, Nature, 427, pp. 355-360, 2004). FTY 720 is a synthetic analog of a natural compound derived from the fungus Isaria sinclairii. FTY 720 induces a decrease in circulating lymphocytes and the efficacy has been attributed to arise from the agonist driven functional mechanism of S1P1. This compound advanced to Phase III clinical trials for the treatment of organ transplantation and in Phase II for multiple sclerosis.

However, FTY 720 is reported to have an adverse event of transient asymptomatic bradycardia (J. Am. Soc. Nephrol., 13, 1073, 2002) and the toxicity potential is mechanism based due to non selective agonism on S1P3 receptor (Bioorg. & Med. Chem. Lett., 14, 3501, 2004).

Recently, further selective agonists of SiP have also been described in the patent literature (WO 04 058149, WO 04 024673, WO 03 061567).

However, FTY 720 and congeners suffer from two potential drawbacks in this indication: they require to be phosphorylated in vivo to become active (Brinkmann et al, J. Biol. Chem., 277, 24, pp. 21453-21457, 2002) and suffer from a lack of selectivity towards the five SIP receptor subtypes (Mandala et al, Science, 296, pp. 346-349, 2002).

There is therefore potential interest in developing direct SIP receptor agonists displaying receptor selectivity, particularly compounds with low relative activity at the S1P3 receptor subtype expressed in cardiac tissues and whose activation results in bradycardia and cardiac depression (Forrest et al, JPET, 309, pp. 758-768, 2004, Sanna et al, J. Biol. Chem., 279 (14), pp. 13839-13848, 2004)

It is thus an object of the present invention to provide compounds that are SIP receptor agonists, having immunosuppressive activity preferably with low affinity to edg3/S1 P3 receptor.

Surprisingly, the present inventors have identified new compounds of formula (I) which fulfill these requirements.

Compounds of close structures have been disclosed (Hayakawa et al, Bulletin of the chemical society of Japan, 46, 6, 1973, 1886-1887; U.S. Pat. No. 3,325,360, Sugasawa et al Pharmaceutical bulletin, Pharmaceutical society of Japan 3, 1, 47-52; GB 917817). Nevertheless, their SP activity is neither taught nor suggested.

According to a first object, the present invention concerns new compounds of formula (I):

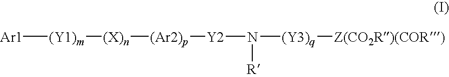

wherein
Ar1 represents an Aryl or Heteroaryl group optionally substituted by 1 up to 5 R group(s),

wherein, each R, identical or different, is selected from the group consisting in Halogen atom, perhalogenoalkyl, -Alkyl, —OAlkyl, —OH, —COOR7, —CONR7R8, -Alkyl-Hal, —OAlkyl-Hal, NO2, —CN, —NR7R8, -AlkylAryl, -Aryl, —S(O)lR7, -Alkenyl, —Si(Alkyl)₃;

wherein R7 and R8, identical or different, are chosen from the group consisting in H; Alkyl; Cycloalkyl; Aryl; -AlkylAryl; Heteroaryl; wherein Alkyl, Cycloalkyl, and/or Aryl is(are) optionally substituted by one or more identical or different Halogen atom, polyfluoroalkyl, Aryl, —COOR7
or R7 and R8 form together with the N atom to which they are attached a Heterocycle;
l is O, 1 or 2
Y1 represents an -Alkyl-chain, optionally substituted by one or more R as defined above, and optionally comprising one or more unsaturation(s) and/or heteroatom(s) and/or a residue chosen from the group consisting in -Alkenyl-, -Alkynyl-, —CH═N—, —N═CH—, —CH═N—O—, —O—N═CH—, —C(═O)—, —C(═O)(OR7)-, —C(═O)(NR7)-, —N═N—, —S(O)l—;
m is 0 or 1;
X represents a heteroatom;
n is 0 or 1;

—Ar2- represents an -Aryl-group optionally substituted by one or more R group as defined above or wherein 2 R may form together with the atoms to which they are attached a fused cyclic, aryl or heteroaryl ring
p is 0 or 1;
—Y2- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH—N—O—, —O—N═CH—, —C(═O)—, —C(═O)O—, —C(═O)(NR7), —N═N—, —S(O)l—;
—R′ represents a hydrogen atom, or a -Cycloalkyl or an -Alkyl chain, the cycloalkyl or alkyl being optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)O—, —C(═O)(NR7)-, —N═N—, —S(O)l—, such as -Alkenyl-Alkyl, -Alkynyl-Alkyl, —CH═N-Alkyl, —N═CH-Alkyl, —CH═N—O-Alkyl, —O—N═CH-Alkyl, —C(═O)-Alkyl, —C(═O)O-Alkyl, —C(═O)(NR7)-Alkyl, —N═N-Alkyl, —S(O)l-Alkyl;
—Y3- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7)-, —N═N—, —S(O)l—;
q is 0 or 1;
Z represents a -Cycloalkyl< group or an -Aryl<, -Heteroaryl< group or a —C(alkyl)< group, or R′ and Z form together with the N atom to which they are attached a Heterocyclic or Heteroaryl group;
   wherein (CO2R″) and (COR′″) can be attached to the same atom or two adjacent atoms;
—R″ represents a hydrogen atom or an Alkyl chain, optionally substituted by one or more R as defined above, and optionally comprising one or more unsaturation(s);
—R′″ represents —OH, —OAlkyl, H, —NR7OR8, —NR7R8, natural or synthetic amino acids, wherein R7 and R8 are defined as above;

or R″ and R′″ form together a ring with the atom(s) to which they are attached to form a cyclic intramolecular bis carbonyl group, including Meldrum acid derivatives;

as well as their enantiomers, diastereoisomers, geometrical isomers, mixtures thereof, free forms and pharmaceutically acceptable salts, hydrates, solvates and esters

with the exception of compounds where:
m=n=p=0, q=0 or 1, R′═H or Alkyl, Y2=-Alkyl-, Y3=-Alkyl-, Z=-C(Alkyl)< and Ar1=unsubstituted phenyl or phenyl substituted with one or more identical R where R is Halogen; and
m=n=p=0, q=1, R′═H, Y2=—CH₂—, Y3=—CH₂—, Z=-cyclobutyl< and Ar1=unsubstituted phenyl.

Preferably, the compounds of the invention are represented by the following general formula (II):

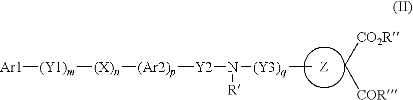

in which Ar1, Y1, X, Ar2, Y2, R, R′, Y3, R″, R′″, m, n, p, q are defined as above and

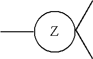

represents a -Cycloalkyl< group or an -Heterocyclic<, -Aryl<, -Heteroaryl< group;
wherein (CO2R″) and (COR′″) can be attached to the same atom or two adjacent atoms of the Z ring;
as well as their enantiomers, diastereoisomers, geometrical isomers, mixtures thereof and pharmaceutically acceptable salts, hydrates, solvates and esters.

Preferably, in the general formula (II):
Ar1 represents an Aryl or Heteroaryl group optionally substituted, preferably, substituted, by 1 up to 5 R group(s),
   wherein, each R, identical or different, is selected from the group consisting in Halogen atom, perhalogenoalkyl, -Alkyl, —OAlkyl,
Y1 represents an -Alkyl-chain or a —S-Alkyl-chain;
m is 0 or 1;
X represents a heteroatom;
n is 0 or 1;
—Ar2- represents an -Aryl-group;
p is 0 or 1;
—Y2- represents an -Alkyl-chain;
—R′ represents a hydrogen atom or a cycloalkyl or an alkyl chain;
q is 0;

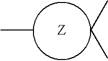

represents a -Cycloalkyl< group and (CO2R″) and (COR′″) are attached to the same atom of the Z ring;
—R″ represents a hydrogen atom;
—R′″ represents —OH, —OAlkyl, —NR7R8.

In the general formula (I) or (II):

Preferably, Ar1 represents a Phenyl or Thienyl group optionally substituted, preferably substituted, by 1 up to 5 R group(s),

wherein, each R identical or different, is selected from the group consisting in Halogen atom, perhalogenoalkyl, -Alkyl, —OAlkyl,

Preferably, Y1 represents an -Alkyl-chain or a —S-Alkyl-chain;

Preferably, m is 0 or 1;

Preferably, X represents an Oxygen atom;

Preferably, n is 0 or 1;

Preferably, —Ar2- represents an -Phenyl-group;

Preferably, p is 0 or 1;

Preferably, —Y2- represents an -Alkyl-chain;

Preferably, —R′ represents a hydrogen atom or a cycloalkyl or an alkyl chain or —COOAlkyl;

Preferably, q is 0 or 1;

Preferably, Y3 represents an alkynyl chain;

Preferably, Z represents a —C(alkyl)< group; or

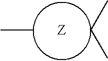

represents a -Cycloalkyl< group, wherein (CO2R″) and (COR′″) are attached to the same atom of the Z ring

Preferably, —R″ represents a hydrogen atom;

Preferably, —R′″ represents —OH, —OAlkyl, —NR7R8

According to a preferred aspect, compounds of the invention are selected from the group consisting in:
3-(4-octylbenzylamino)cyclopentane-1,1-dicarboxylic acid
3-(4-octylbenzylamino)cyclopentane-1,1-dicarboxylic acid ethyl ester
3-(4-nonylbenzylamino)cyclopentane-1,1-dicarboxylic acid
3-(4-octyloxybenzylamino)cyclopentane-1,1-dicarboxylic acid
3-(3-methoxy-4-octyloxybenzylamino)cyclopentane-1,1-dicarboxylic acid
3-(3-methoxy-4-butyloxybenzylamino)cyclopentane-1,1-dicarboxylic acid
3-(3-methoxy-4-hexylyloxybenzylamino)cyclopentane-1,1-dicarboxylic acid
3-(3-bromo-4-octyloxy-5-methoxybenzylamino)cyclopentane-1,1-dicarboxylic acid
3-(3-methoxy-4-octyloxy-5-methylbenzylamino)cyclopentane-1,1-dicarboxylic acid
3-(3-chloro-4-octyloxy-5-methoxybenzylamino)cyclopentane-1,1-dicarboxylic acid
3-(4-nonyloxybenzylamino)cyclopentane-1,1-dicarboxylic acid
3-(4-decylbenzylamino)cyclopentane-1,1-dicarboxylic acid
3-(3-methoxy-4-nonyloxybenzylamino)cyclopentane-1,1-dicarboxylic acid
3-(3-chloro-4-nonyloxybenzylamino)cyclopentane-1,1-dicarboxylic acid
3-[4-[4-(4-methoxyphenyl)butoxy]benzylamino]cyclopentane-1,1-dicarboxylic acid
3-[4-[4-(3-chloro-4-methoxyphenyl)butoxy]benzylamino]-cyclopentane-1,1-dicarboxylic acid
3-(4-nonylbenzylamino)cyclopentane-1,1-dicarboxylic acid ethyl ester hydrochloride
3-{4-[3-fluoro-(4-hexylphenyl)benzyl]amino}cyclopentane-1,1-dicarboxylic acid hydrochloride
1-(2,2,2-trifluoroethylcarbamoyl)-3-(4-nonylbenzylamino)cyclopentanecarboxylc acid hydrochloride (isomer A)
1-(2,2,2-trifluoroethylcarbamoyl)-3-(4-nonylbenzylamino)cyclopentanecarboxylic acid hydrochloride (isomer B)
3-(4-decyloxybenzylamino)cyclopentane-1,1-dicarboxylic acid hydrochloride
1-(methylcarbamoyl)-3-(4-nonylbenzylamino)cyclopentanecarboxylic acid hydrochloride (isomer A)
1-(methylcarbamoyl)-3-(4-nonylbenzylamino)cyclopentanecarboxylic acid hydrochloride (isomer B)
3-(methyl-4-nonylbenzylamino)cyclopentane-1,1-dicarboxylic acid hydrochloride
3-[4-(4-phenylsulfanylbutoxy)benzylamino]cyclopentane-1,1-dicarboxylic acid hydrochloride
3-{4-[4-(4-ethoxy-3-chlorophenyl)butoxy]benzylamino}cyclopentane-1,1-dicarboxylic acid hydrochloride
2-methyl-2-[4-(4-nonylbenzylamino)but-2-ynyl]malonic acid hydrochloride
2-[4-(3-chloro-4-nonyloxybenzylamino)but-2-ynyl]-2-methylmalonic acid hydrochloride
2-methyl-2-[4-(4-octylbenzylamino)but-2-ynyl]malonic acid hydrochloride
2-[4-(4-decylbenzylamino)but-2-ynyl]-2-methylmalonic acid hydrochloride
2-[3-fluoro-4-(4-hexylphenyl)benzylamino]but-2-ynyl]-2-methylmalonic acid hydrochloride
3-[(2-Fluoro-4′-heptylbiphenyl-4-ylmethyl)amino]cyclopentane-1,1-dicarboxylic acid
1-Carbamoyl-3-(4-nonylbenzylamino)cyclopentanecarboxylic acid—Isomer B 3-(3-Chloro-4-decyloxybenzylamino)cyclopentane-1,1-dicarboxylic acid
3-(3-Chloro-4-octyloxybenzylamino)cyclopentane-1,1-dicarboxylic acid
1-Carbamoyl-3-(4-nonylbenzylamino)cyclopentanecarboxylic acid—Isomer A 3-[Cyclopropyl-(4-nonylbenzyl)amino]cyclopentane-1,1-dicarboxylic acid
3-[(2-Fluoro-4′-heptylbiphenyl-4-ylmethyl)methylamino]cyclopentane-1,1-dicarboxylic acid
3-(2,3-Difluoro-4-nonylbenzylamino)cyclopentane-1,1-dicarboxylic acid
3-(2,3-Difluoro-4-nonylbenzylamino)cyclopentane-1,1-dicarboxylic acid ethyl ester
3-(4-Nonyl-benzylamino)cyclopentane-1,1-dicarboxylic acid isobutyl ester
3-[Methyl-(4-nonylbenzyl)amino]cyclopentane-1,1-dicarboxylic acid ethyl ester
3-(4-Nonylbenzylamino)cyclopentane-1,1-dicarboxylic acid methyl ester
3-(4-Nonylbenzylamino)cyclopentane-1,1-dicarboxylic acid benzyl ester
3-[(2-Fluoro-4′-octylbiphenyl-4-ylmethyl)amino]cyclopentane-1,1-dicarboxylic acid ethyl ester
3-[(2-Fluoro-4′-octylbiphenyl-4-ylmethyl)amino]cyclopentane-1,1-dicarboxylic acid
3-[Cyclopropyl-(2-fluoro-4′-heptylbiphenyl-4-ylmethyl)amino]cyclopentane-1,1-dicarboxylic acid
3-(4-Nonylbenzylamino)cyclopentane-1,1-dicarboxylic acid isopropylester
3-(2,3-Difluoro-4-nonylbenzylamino)-1-methylcarbamoylcyclopentane carboxylic acid—Isomer A
3-(2,3-Difluoro-4-nonylbenzylamino)-1-methylcarbamoylcyclopentane carboxylic acid—Isomer B
3-(4-Nonylbenzylamino)-1-propylcarbamoylcyclopentanecarboxylic acid—Isomer A
3-(4-Nonylbenzylamino)-1-propylcarbamoylcyclopentanecarboxylic acid—Isomer B
3-(4-Decyl-2,3-difluorobenzylamino)cyclopentane-1,1-dicarboxylic acid
3-(4-Decylbenzylamino)-1-methylcarbamoylcyclopentanecarboxylic acid-Isomer A
3-(4-Decylbenzylamino)-1-methylcarbamoylcyclopentanecarboxylic acid—Isomer B
1-Ethylcarbamoyl-3-(4-nonylbenzylamino)cyclopentanecarboxylic acid—Isomer A
1-Ethylcarbamoyl-3-(4-nonylbenzylamino)cyclopentanecarboxylic acid—Isomer B
1-Isopropylcarbamoyl-3-(4-nonylbenzylamino)cyclopentanecarboxylic acid—Isomer A
1-Isopropylcarbamoyl-3-(4-nonylbenzylamino)cyclopentanecarboxylic acid—Isomer B
3-(4-Decyl-2,3-difluorobenzylamino)-1-methylcarbamoylcyclopentanecarboxylic acid—Isomer A
3-[(2,3-Difluoro-4-nonylbenzyl)methylamino]-1-methylcarbamoylcyclopentanecarboxylic acid—Isomer A
3-[(2,3-Difluoro-4-nonylbenzyl)methylamino]-1-methylcarbamoyl-cyclopentanecarboxylic acid—Isomer B
3-[(4-Decylbenzyl)methylamino]cyclopentane-1,1-dicarboxylic acid
3-(4-Nonylbenzylamino)cyclopentane-1,1-dicarboxylic acid butyl ester
3-(4-Nonylbenzylaminocyclopentane-1,1-dicarboxylic acid propyl ester 1-(3,4-Difluorophenyl carbamoyl)-3-(4-nonylbenzylamino)cyclopentanecarboxylic acid
3-[(2,3-Difluoro-4-nonylbenzyl)methylamino]cyclopentane-1,1-dicarboxylic acid
3-(4-Decyl-2,3-difluorobenzylamino)cyclopentane-1,1-dicarboxylic acid isopropyl ester
3-(4-Decylbenzylamino)cyclopentane-1,1-dicarboxylic acid isopropyl ester
3-[(2-Fluoro-4′-heptylbiphenyl-4-ylmethyl)amino]-1-methylcarbamoylcyclopentanecarboxylic acid—Isomer A
3-[(2-Fluoro-4′-heptylbiphenyl-4-ylmethyl)amino]-1-methylcarbamoylcyclopentanecarboxylic acid—Isomer B
3-(4-Decylbenzylamino)cyclopentane-1,1-dicarboxylic acid enantiomer A
3-(4-Decylbenzylamino)cyclopentane-1,1-dicarboxylic acid enantiomer B
3-[(3-Chloro-4-decyloxybenzyl)methylamino]cyclopentane-1,1-dicarboxylic acid
3-[(3-Chloro-4-nonylloxybenzyl)methylamino]cyclopentane-1,1-dicarboxylic acid
1-Dimethylcarbamoyl-3-(4-nonylbenzylamino)cyclopentanecarboxylic acid
1-Ethylcarbamoyl-3-(4-octylbenzylamino)cyclopentanecarboxylic acid—Isomer A
1-Ethylcarbamoyl-3-(4-octylbenzylamino)cyclopentanecarboxylic acid—Isomer B
1-Methylcarbamoyl-3-(4-octylbenzylamino)cyclopentanecarboxylic acid—Isomer A
1-Methylcarbamoyl-3-(4-octylbenzylamino)cyclopentanecarboxylic acid—Isomer B
3-(4-octylbenzylamino)cyclopentane-1,1-dicarboxylic acid isopropyl ester
3-[(4-Decyl-2,3-difluorobenzyl)methylamino]cyclopentane-1,1-dicarboxylic acid
3-(4-Decyl-2,3-difluorobenzylamino)cyclopentane-1,1-dicarboxylic acid enantiomer B
3-(4-Decyl-2,3-difluorobenzylamino)cyclopentane-1,1-dicarboxylic acid enantiomer A
3-[(2-Fluoro-4′-heptylbiphenyl-4-ylmethyl)amino]cyclopentane-1,1-dicarboxylic acid enantiomer B
3-(4-Decylbenzylamino)cyclopentane-1,1-dicarboxylic acid ethyl ester—Isomer A
3-(4-Decylbenzylamino)cyclopentane-1,1-dicarboxylic acid ethyl ester—Isomer B
3-[(4-Decylbenzyl)methylamino]cyclopentane-1,1-dicarboxylic acid ethyl ester
3-[(4-Decyl-2,3-difluorobenzyl)methylamino]cyclopentane-1,1-dicarboxylic acid enantiomer B
3-[(4-Decyl-2,3-difluorobenzyl)methylamino]cyclopentane-1,1-dicarboxylic acid ethyl ester enantiomer B
3-[(4-Decyl-2,3-difluorobenzyl)methylamino]cyclopentane-1,1-dicarboxylic acid enantiomer A
3-[(4-Decyl-2,3-difluorobenzyl)methylamino]cyclopentane-1,1-dicarboxylic acid ethyl ester enantiomer A
3-(4-Decyl-2,6-difluorobenzylamino)cyclopentane-1,1-dicarboxylic acid
3-[(4-Decyl-2,6-difluorobenzyl)methylamino]cyclopentane-1,1-dicarboxylic acid
3-[(4-Decyl-2,3-difluorobenzyl)ethoxycarbonylamino]cyclopentane-1,1-dicarboxylic acid
3-[(4-Decyl-2,3-difluorobenzyl)ethoxycarbonylamino]cyclopentane-1,1-dicarboxylic acid ethyl ester—Isomer A
3-[(4-Decyl-2,3-difluorobenzyl)ethoxycarbonylamino]cyclopentane-1,1-dicarboxylic acid ethyl ester—Isomer B
3-[(4-Decyl-2,3-difluorobenzyl)methoxycarbonylamino]cyclopentane-1,1-dicarboxylic acid
3-[(4-Decyl-2,3-difluorobenzyl)methoxycarbonylamino]cyclopentane-1,1-dicarboxylic acid ethyl ester
2-Methyl-2-[4-(4-nonylbenzylamino)but-2-enyl]malonic acid
3-[2-(4-Octylphenyl)ethylamino]cyclopentane-1,1-dicarboxylic acid
1-(1-Carboxy-2-phenylethylcarbamoyl)-3-(4-nonylbenzylamino)cyclopentane-carboxylic acid
3-{4-[5-(3-Chloro-4-ethoxyphenyl)pentyloxy]benzylamino}cyclopentane-1,1-dicarboxylic acid
3-[4-(4-Phenyl-5-trifluoromethylthiophen-2-ylmethoxy)benzylamino]cyclopentane-1,1-dicarboxylic acid enantiomer B
3-{Methyl-[4-(4-phenyl-5-trifluoromethylthiophen-2-ylmethoxy)benzyl]amino}cyclopentane-1,1-dicarboxylic acid
3-[(2-Fluoro-4′-heptylbiphenyl-4-ylmethyl)amino]cyclobutane-1,1-dicarboxylic acid
3-(4-Nonylbenzylamino)cyclobutane-1,1-dicarboxylic acid
3-(4-Decylbenzylamino)cyclobutane-1,1-dicarboxylic acid
3-[(4-Decylbenzyl)methylamino]cyclobutane-1,1-dicarboxylic acid
3-(4-Decyl-2,3-difluorobenzylamino)cyclobutane-1,1-dicarboxylic acid
3-[(2-Fluoro-4′-heptylbiphenyl-4-ylmethyl)amino]-1-methylcarbamoylcyclobutanecarboxylic acid—Isomer A
3-[(2-Fluoro-4′-heptylbiphenyl-4-ylmethyl)amino]-1-methylcarbamoylcyclobutanecarboxylic acid—Isomer B
3-[(4-Decylbenzyl)methylamino]cyclobutane-1,1-dicarboxylic acid ethyl ester
3-[(4-Decyl-2,3-difluorobenzyl)methylamino]cyclobutane-1,1-dicarboxylic acid
3-[(4-Decyl-2,3-difluorobenzyl)methylamino]cyclobutane-1,1-dicarboxylic acid ethyl ester
3-[4-(4-Phenyl-5-trifluoromethylthiophen-2-ylmethoxy)benzylamino]cyclobutane-1,1-dicarboxylic acid
1-(4-Decylbenzyl)piperidine-4,4-dicarboxylic acid
1-(4-Nonylbenzyl)piperidine-4,4-dicarboxylic acid
1-(4-Decyl-2,3-difluorobenzyl)piperidine-4,4-dicarboxylic acid
1-(4-Octylbenzyl)piperidine-4,4-dicarboxylic acid
1-(3-Chloro-4-nonyloxybenzyl)piperidine-4,4-dicarboxylic acid
1-(2-Fluoro-4′-heptylbiphenyl-4-ylmethyl)piperidine-4,4-dicarboxylic acid
1-(4-Decylbenzyl)piperidine-4,4-dicarboxylic acid ethyl ester
1-(3-Chloro-4-decyloxybenzyl)piperidine-4,4-dicarboxylic acid ethyl ester
1-(4-Decyl-2,3-difluorobenzyl)piperidine-4,4-dicarboxylic acid ethyl ester
1-(2-Fluoro-4′-heptylbiphenyl-4-ylmethyl)piperidine-4,4-dicarboxylic acid ethyl ester
1-(4-Decyl-2,3-difluorobenzyl)piperidine-4,4-dicarboxylic acid methyl ester
1-(4-Decyl-2,3-difluorobenzyl)piperidine-4,4-dicarboxylic acid isopropyl ester
1-(4-Decylbenzyl)-4-ethylcarbamoylpiperidine-4-carboxylic acid
1-(4-Decylbenzyl)-4-propylcarbamoylpiperidine-4-carboxylic acid
1-(2,3-difluoro-4-nonylbenzyl)-4-ethylcarbamoylpiperidine-4-carboxylic acid
4-[(4-Decyl-2,3-difluorobenzyl)methylamino]cyclohexane-1,1-dicarboxylic acid diethyl ester
4-[(2,3-Difluoro-4-nonylbenzyl)methylamino]cyclohexane-1,1-dicarboxylic acid diethyl ester
4-(2,3-Difluoro-4-nonylbenzylamino)cyclohexane-1,1-dicarboxylic acid
4-[(2,3-Difluoro-4-nonylbenzyl)methylamino]cyclohexane-1,1-dicarboxylic acid ethyl ester
4-[(4-Decyl-2,3-difluorobenzyl)methylamino]cyclohexane-1,1-dicarboxylic acid ethyl ester
4-(4-Decyl-2,3-difluorobenzylamino)cyclohexane-1,1-dicarboxylic acid
4-[(2,3-Difluoro-4-nonylbenzyl)methylamino]cyclohexane-1,1-dicarboxylic acid
4-[(4-Decyl-2,3-difluorobenzyl)methylamino]cyclohexane-1,1-dicarboxylic acid
1-[4-(4-Phenyl-5-trifluoromethylthiophen-2-ylmethoxy)benzyl]-piperidine-4,4-dicarboxylic acid
1-(4-Decylbenzyl)azetidine-3,3-dicarboxylic acid
2-[4-(3-Chloro-4-decyloxybenzylamino)but-2-ynyl]-2-methylmalonic acid
2-{4-[(2-Fluoro-4′-heptylbiphenyl-4-ylmethyl)amino]but-2-ynyl}-2-methylmalonic acid
2-{4-[Isopropyl(4-nonylbenzyl)amino]but-2-ynyl}-2-methylmalonic acid
2-{4-[Cyclopropyl(4-nonylbenzyl)amino]but-2-ynyl}-2-methylmalonic acid
2-{4-[(2-Fluoro-4′-hexylbiphenyl-4-ylmethyl)methylamino]but-2-ynyl}-2-methylmalonic acid
2-Methyl-2-{4-[methyl(4-nonylbenzyl)amino]but-2-ynyl}malonic acid
2-{4-[(2,3-Difluoro-4-nonylbenzyl)methylamino]but-2-ynyl}-2-methylmalonic acid.
2-{4-[Ethyl-(4-nonylbenzyl)amino]but-2-ynyl}-2-methylmalonic acid
2-{4-[(2,3-Difluoro-4-nonylbenzyl)amino]but-2-ynyl}-2-methylmalonic acid
2-[(4-(4-Decyl-2,3-difluorobenzylamino)but-2-ynyl]-2-methylmalonic acid
2-{4-[(4-Decylbenzyl)methylamino]but-2-ynyl}-2-methylmalonic acid
2-{4-[(3-Chloro-4-nonyloxybenzyl)methylamino]but-2-ynyl}-2-methylmalonic acid.
2-Ethyl-2-{4-[(2-fluoro-4′-hexylbiphenyl-4-ylmethyl)amino]but-2-ynyl}malonic acid
2-Methyl-2-{4-[(4-(4-phenyl-5-trifluoromethylthiophen-2-ylmethoxy)benzylamino]but-2-ynyl}malonic acid
   as well as their enantiomers, diastereoisomers, geometrical isomers, mixtures thereof, free forms and pharmaceutically acceptable salts, hydrates, solvates and esters.

According to a still preferred aspect, compounds of the invention are selected from the group consisting in:
3-(4-nonylbenzylamino)cyclopentane-1,1-dicarboxylic acid
3-(4-decylbenzylamino)cyclopentane-1,1-dicarboxylic acid
3-(methyl-4-nonylbenzylamino)cyclopentane-1,1-dicarboxylic acid hydrochloride
3-(2,3-Difluoro-4-nonylbenzylamino)cyclopentane-1,1-dicarboxylic acid
3-[Methyl-(4-nonylbenzyl)amino]cyclopentane-1,1-dicarboxylic acid ethyl ester
3-(4-Decyl-2,3-difluorobenzylamino)cyclopentane-1,1-dicarboxylic acid
3-[(2,3-Difluoro-4-nonylbenzyl)methylamino]cyclopentane-1,1-dicarboxylic acid
3-[(4-Decyl-2,3-difluorobenzyl)methylamino]cyclopentane-1,1-dicarboxylic acid
3-[(4-Decyl-2,3-difluorobenzyl)methylamino]cyclopentane-1,1-dicarboxylic acid enantiomer B
3-[(4-Decyl-2,3-difluoro-benzyl)methyl-amino]-cyclopentane-1,1-dicarboxylic acid ethyl ester enantiomer B
3-[(4-Decyl-2,6-difluorobenzyl)methylamino]cyclopentane-1,1-dicarboxylic acid
3-[(4-Decyl-2,3-difluorobenzyl)methylamino]cyclobutane-1,1-dicarboxylic acid
3-[(4-Decyl-2,3-difluorobenzyl)methylamino]cyclobutane-1,1-dicarboxylic acid ethyl ester
1-(4-Decyl-2,3-difluorobenzyl)piperidine-4,4-dicarboxylic acid
1-(2-Fluoro-4′-heptylbiphenyl-4-ylmethyl)piperidine-4,4-dicarboxylic acid
1-(4-Decyl-2,3-difluorobenzyl)piperidine-4,4-dicarboxylic acid ethyl ester
1-(4-Decyl-2,3-difluorobenzyl)piperidine-4,4-dicarboxylic acid methyl ester
1-(4-Decyl-2,3-difluorobenzyl)piperidine-4,4-dicarboxylic acid isopropyl ester
1-(2,3-difluoro-4-nonylbenzyl)-4-ethylcarbamoyl piperidine-4-carboxylic acid
   as well as their enantiomers, diastereoisomers, geometrical isomers, mixtures thereof, free forms and pharmaceutically acceptable salts, hydrates, solvates and esters.

As used hereabove or hereafter:

Alk refers to Alkyl, Alkenyl or Alkynyl.

“Alkyl” means an aliphatic hydrocarbon group which may be straight or branched having 1 to 20 carbon atoms in the chain. Preferred alkyl groups have 1 to 12 carbon atoms in the chain. Branched means that one or more lower alkyl groups such as methyl, ethyl or propyl are attached to a linear alkyl chain. Exemplary alkyl groups include methyl, ethyl, n-propyl, i-propyl, n-butyl, t-butyl, n-pentyl, 3-pentyl, octyl, nonyl, decyl.

“Alkenyl” means an aliphatic hydrocarbon group containing a carbon-carbon double bond and which may be straight or branched having 2 to 15 carbon atoms in the chain. Preferred alkenyl groups have 2 to 12 carbon atoms in the chain; and more preferably about 2 to 4 carbon atoms in the chain. Exemplary alkenyl groups include ethenyl, propenyl, n-butenyl, i-butenyl, 3-methylbut-2-enyl, n-pentenyl, heptenyl, octenyl, nonenyl, decenyl.

“Alkynyl” means an aliphatic hydrocarbon group containing a carbon-carbon triple bond and which may be straight or branched having 2 to 15 carbon atoms in the chain. Preferred alkynyl groups have 2 to 12 carbon atoms in the chain; and more preferably 2 to 4 carbon atoms in the chain. Exemplary alkynyl groups include ethynyl, propynyl, n-butynyl, 2-butynyl, 3-methylbutynyl, n-pentynyl, heptynyl, octynyl and decynyl.

“Halogen atom” refers to fluorine, chlorine, bromine or iodine atom; preferably fluorine and chlorine atom.

“Cycloalkyl” means a non-aromatic mono- or multicyclic hydrocarbon ring system of 3 to 10 carbon atoms, preferably of 4 to 10 carbon atoms. Preferred ring sizes of rings of the ring system include 4 to 6 ring atoms. Exemplary monocyclic cycloalkyl include cyclobutyl, cyclopentyl, cyclohexyl, cycloheptyl, and the like. Exemplary multicyclic cycloalkyl include 1-decalin, norbornyl, adamant-(1- or 2-)yl.

“Aryl” means an aromatic monocyclic or multicyclic hydrocarbon ring system of 6 to 14 carbon atoms, preferably of 6 to 10 carbon atoms. Exemplary aryl groups include phenyl or naphthyl.

As used herein, the terms “heterocycle” or “heterocyclic” refer to a saturated, partially unsaturated or unsaturated, non aromatic stable 3 to 14, preferably 5 to 10 membered mono, bi or multicyclic rings wherein at least one member of the ring is a hetero atom. Typically, heteroatoms include, but are not limited to, oxygen, nitrogen, sulfur, selenium, and phosphorus atoms. Preferable heteroatoms are oxygen, nitrogen and sulfur.

Suitable heterocycles are also disclosed in The Handbook of Chemistry and Physics, 76th Edition, CRC Press, Inc., 1995-1996, pages 2-25 to 2-26, the disclosure of which is hereby incorporated by reference.

Preferred non aromatic heterocyclic include, but are not limited to oxiranyl, tetrahydrofuranyl, dioxolanyl, tetrahydropyranyl, dioxanyl, pyrrolidinyl, piperidyl, morpholinyl, imidazolidinyl, pyranyl, imidazolinyl, pyrrolinyl, pyrazolinyl.

Preferred aromatic heterocyclic, herein called heteroaryl groups include, but are not limited to, pyridyl, pyridyl-N-oxide, pyrimidinyl, pyrrolyl, furanyl, thienyl, imidazolyl, triazolyl, tetrazolyl, quinolyl, isoquinolyl, benzoimidazolyl, thiazolyl, pyrazolyl, and benzothiazolyl groups.

As used herein, the term “heteroaryl” refers to a 5 to 14, preferably 5 to 10 membered aromatic hetero, mono-, bi- or multicyclic ring. Examples include pyrrolyl, pyridyl, pyrazolyl, thienyl, pyrimidinyl, pyrazinyl, tetrazolyl, indolyl, quinolinyl, purinyl, imidazolyl, thienyl, thiazolyl, benzothiazolyl, furanyl, benzofuranyl, 1,2,4-thiadiazolyl, isothiazolyl, triazoyl, tetrazolyl, isoquinolyl, benzothienyl, isobenzofuryl, pyrazolyl, carbazolyl, benzimidazolyl, isoxazolyl.

“Alkyl”, “cycloalkyl”, “alkenyl”, “alkynyl”, “aryl”, “heteroaryl”, “heterocycle” refers also to the corresponding “alkylene”, “cycloalkylene”, “alkenylene”, “alkynylene”, “arylene”, “heteroarylene”, “heterocyclene” which are formed by the removal of two hydrogen atoms.

As used herein, the term “patient” refers to a warm-blooded animal such as a mammal, preferably a human or a human child, which is afflicted with, or has the potential to be afflicted with one or more diseases and conditions described herein.

As used herein, a “therapeutically effective amount” refers to an amount of a compound of the present invention which is effective in reducing, eliminating, treating or controlling the symptoms of the herein-described diseases and conditions. The term “controlling” is intended to refer to all processes wherein there may be a slowing, interrupting, arresting, or stopping of the progression of the diseases and conditions described herein, but does not necessarily indicate a total elimination of all disease and condition symptoms, and is intended to include prophylactic treatment and chronic use.

As used herein, the term “pharmaceutically acceptable” refers to those compounds, materials, compositions, or dosage forms which are, within the scope of sound medical judgment, suitable for contact with the tissues of human beings and animals without excessive toxicity, irritation, allergic response, or other problem complications commensurate with a reasonable benefit/risk ratio.

As used herein, “pharmaceutically acceptable salts” refer to derivatives of the disclosed compounds wherein the parent compound is modified by making acid or base salts thereof. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, tartaric, citric, methanesulfonic, benzenesulfonic, glucoronic, glutamic, benzoic, salicylic, toluenesulfonic, oxalic, fumaric, maleic, and the like. Further addition salts include ammonium salts such as tromethamine, meglumine, epolamine, etc., metal salts such as sodium, potassium, calcium, zinc or magnesium.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two. Generally, non-aqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418 and P. H. Stahl, C. G. Wermuth, Handbook of Pharmaceutical salts—Properties, Selection, and Use Wiley-VCH, 2002, the disclosures of which are hereby incorporated by reference.

The compounds of the general formula (I) having geometrical and stereoisomers are also a part of the invention.

According to a further object, the present invention is also concerned with the process of preparation of the compounds of formula (I).

The compounds and process of the present invention may be prepared in a number of ways well known to those skilled in the art. The compounds can be synthesized, for example, by application or adaptation of the methods described below, or variations thereon as appreciated by the skilled artisan. The appropriate modifications and substitutions will be readily apparent and well known or readily obtainable from the scientific literature to those skilled in the art.

In particular, such methods can be found in R. C. Larock, Comprehensive Organic Transformations, VCH publishers, 1989.

It will be appreciated that the compounds of the present invention may contain one or more asymmetrically substituted carbon atoms, and may be isolated in optically active or racemic forms. Thus, all chiral, diastereomeric, racemic forms and all geometric isomeric forms of a structure are intended, unless the specific stereochemistry or isomeric form is specifically indicated. It is well known in the art how to prepare and isolate such optically active forms. For example, mixtures of stereoisomers may be separated by standard techniques including, but not limited to, resolution of racemic forms, normal, reverse-phase, and chiral chromatography, preferential salt formation, recrystallization, and the like, or by chiral synthesis either from chiral starting materials or by deliberate synthesis of target chiral centers.

Compounds of the present invention may be prepared by a variety of synthetic routes. The reagents and starting materials are commercially available, or readily synthesized by well-known techniques by one of ordinary skill in the arts. All substituents, unless otherwise indicated, are as previously defined.

In the reactions described hereinafter it may be necessary to protect reactive functional groups, for example hydroxy, amino, imino, thio or carboxy groups, where these are desired in the final product, to avoid their unwanted participation in the reactions. Conventional protecting groups may be used in accordance with standard practice, for examples see T. W. Greene and P. G. M. Wuts in Protective Groups in Organic Chemistry, John Wiley and Sons, 1991; J. F. W. McOmie in Protective Groups in Organic Chemistry, Plenum Press, 1973.

Some reactions may be carried out in the presence of a base. There is no particular restriction on the nature of the base to be used in this reaction, and any base conventionally used in reactions of this type may equally be used here, provided that it has no adverse effect on other parts of the molecule. Examples of suitable bases include: sodium hydroxide, potassium carbonate, triethylamine, alkali metal hydrides, such as sodium hydride and potassium hydride; alkyllithium compounds, such as methyllithium and butyllithium; and alkali metal alkoxides, such as sodium methoxide and sodium ethoxide.

Usually, reactions are carried out in a suitable solvent. A variety of solvents may be used, provided that it has no adverse effect on the reaction or on the reagents involved. Examples of suitable solvents include: hydrocarbons, which may be aromatic, aliphatic or cycloaliphatic hydrocarbons, such as hexane, cyclohexane, benzene, toluene and xylene; amides, such as dimethylformamide; alcohols such as ethanol and methanol and ethers, such as diethyl ether and tetrahydrofuran.

The reactions can take place over a wide range of temperatures. In general, we find it convenient to carry out the reaction at a temperature of from 0° C. to 150° C. (more preferably from about room temperature to 100° C.). The time required for the reaction may also vary widely, depending on many factors, notably the reaction temperature and the nature of the reagents. However, provided that the reaction is effected under the preferred conditions outlined above, a period of from 3 hours to 20 hours will usually suffice.

The compound thus prepared may be recovered from the reaction mixture by conventional means. For example, the compounds may be recovered by distilling off the solvent from the reaction mixture or, if necessary after distilling off the solvent from the reaction mixture, pouring the residue into water followed by extraction with a water-immiscible organic solvent and distilling off the solvent from the extract. Additionally, the product can, if desired, be further purified by various well known techniques, such as recrystallization, reprecipitation or the various chromatography techniques, notably column chromatography or preparative thin layer chromatography.

According to an object of the invention, the process of preparation of a compound of formula (I) of the invention comprises the step of reacting a compound of formula (III):

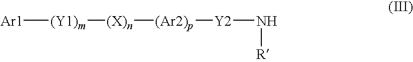

with a compound of formula (IV):

O═(Y3′)_{q}-Z(CO₂R″)(COR′″) (IV)

wherein Ar1, Y1, X, Ar2, Y2, R′, Z, R″, R′″, m, n, p, q are defined as in formula (I), provided that when q=0, the keto function is attached to a carbon atom of Z, and when q=1, Y3′ is such that —CH2-Y3′- corresponds to Y3 as defined in formula (I).

Generally, this coupling reaction is performed out under reductive amination conditions. Preferably, this reaction is carried out by mixing compound (III), optionally in a suitable solvent such as dichloromethane, an ester, preferably ethyl acetate or isopropylacetate, an alcohol preferably ethanol and methanol, or an ether preferably diethyl oxide and tetrahydrofurane, with compound (IV), optionally in a suitable solvent such as dichloromethane, an ester preferably ethyl acetate and isopropylacetate, an alcohol preferably ethanol and methanol, or an ether preferably diethyl oxide and tetrahydrofuran, preferably at room temperature. Then, a suitable reductive agent, such as sodium cyanoborohydride or similar agents such as sodium triacetoxyborohydride, sodium borohydride, tetramethylammonium triacetoxyborohydride, borane-pyridine, dimethylamine-borane, triethylamine-borane, tetrahydrofuran-borane, dimethylsulfane-borane, dimethylaniline-borane, diethylaniline-borane is added with eventually a Bronsted or Lewis acid such as hydrochloric acid, acetic acid, trifluoroacetic acid, titanium tetrachloride, zinc chloride and zinc trifluoroacetate, preferably at temperature comprised between 0° C. and room temperature, more preferably between 5 and 10° C., and the reaction mixture is then allowed to react for a sufficient time to obtain a satisfactory rate.

If necessary, the coupling reaction is followed by further modifying Ar1, Y1, X, Ar2, Y2, R′, Z, R″, R′″ of the compound of formula (I) obtained so as to obtain the desired compound of formula (I). For example, if in the obtained compound of formula (I), R″ represents an alkyl group or R′″ represents a —Oalkyl group, the desired compound of formula (I) in which R″ represents a hydrogen atom or R′″ represents a —OH group can be obtained by saponifying the obtained compound of formula (I). This reaction can be conducted once or twice depending on the number of ester groups to be saponified in the obtained compound of formula (I). This reaction is well known by the skilled person and can be generally conducted under usual conditions, such as in the presence of a base (such as sodium hydroxide or any other suitable base), in a solvent such as an alcohol, including ethanol.

According to another aspect of the invention, the process of preparation of a compound of formula (I) of the invention comprises the step of reacting a compound of formula (V):

Ar1-(Y1)ₘ-(X)ₙ-(Ar2)ₚ-Y2-OSO2Alkyl (V)

with a compound of formula (IX):

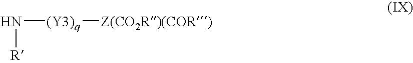

wherein Ar1, Y1, X, Ar2, Y2, R′, Y3, Z, R″, R′″, m, n, p, q are defined as in formula (I).

Generally, the coupling reaction is performed under nucleophilic substitution condition. Preferably, this reaction is carried out by mixing compounds (V) and (IX) in a suitable solvent such as N,N-dimethylformamide, dichloromethane or ethanol in the presence of a base such as N,N-diisopropylethylamine, a carbonate or a bicarbonate preferably at a temperature comprised between room temperature and refluxing temperature.

According to a still further aspect of the invention, the process of preparation of a compound of formula (I) of the invention comprises the step of reacting a compound of formula (VII):

Ar1-(Y1)ₘ-(X)ₙ-(Ar2)ₚ-Y2=O (VII)

with a compound of formula (X):

P H2N(Y3′)q-Z-(CO2R″)(COR′″) (X)

wherein Ar1, Y1, X, Ar2, Y2, Z, R″, R′″, m, n, p, q are defined as in formula (I), Y3′ is such that —CH2-Y3′- corresponds to Y3 as defined in formula (I).

Generally, the coupling reaction is performed under reductive conditions, such as in the presence of sodiumcyanoborohydride.

Further, the process of the invention may also comprise the additional step of isolating the compound of formula (I). This can be done by the skilled person by any of the known conventional means, such as the recovery methods described above.

The compound of formula (III) may be obtained by reacting a compound of formula (V):

Ar1-(Y1)ₘ-(X)ₙ-(Ar2)ₚ-Y2-OSO2Alkyl (V)

with ammonia, such as methanolic ammonia, optionally followed by alkylation of the amino group with an aldehyde under reductive amination conditions such as those described in Organic Reactions, Vol 59, J. Wiley & sons, 2002 or by alkylating a compound of formula (V) with an amine R′NH2 so as to form the —NHR′ group desired.

The compound of formula (V) is in turn obtained by reacting a compound of formula (VI):

Ar1-(Y1)ₘ-(X)ₙ-(Ar2)ₚ-Y2-OH (VI)

with a suitable alkyl sulfonyl chloride derivative, preferably under basic conditions, preferably in the presence of an organic base such as triethylamine or similar.

The compound of formula (VI) may obtained from the corresponding aldehyde derivative (VII):

Ar1-(Y1)ₘ-(X)ₙ-(Ar2)ₚ-Y2=O (VII)

by reducing the aldehyde into the desired alcohol function.

The compound of formula (VII) is commercially available or may be obtained by applying or adapting any known methods or those described in the examples to obtain the desired compound of formula (VII) from available starting products.

The compound of formula (IV) may be obtained from a compound of formula (VIII):

HO—(Y3′)_{q}-Z(CO₂R″)(COR′″) (VIII)

under oxidizing conditions with a chromium oxide derivative such as Jones reagent and pyridinium chlorochromate, activated dimethylsulfoxide reagents such as Swern and Moffatt, sodium or calcium hypochlorite, sodium periodate with a ruthenium salt, sodium permanganate, potassium permanganate, in an inert solvent such as dichloromethane, acetone, acetonitrile, an alkane preferably cyclohexane, hexane and heptane, an ester preferably ethyl acetate and isopropyl acetate, or a mixture of these.

The compound of formula (VIII) is commercially available or may be obtained by applying or adapting any known methods or those described in the examples to obtain the desired compound of formula (VIII) from available starting products.

Of course, the compounds of the present invention can also be obtained in various procedures involving the components of the general formula A, B and C as follows.

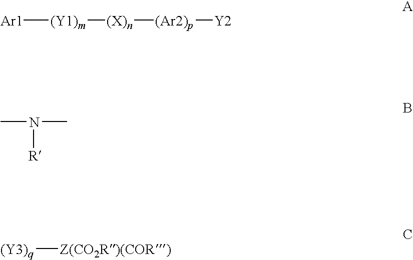

The amine component may be made as part of component A to form AB or with component C to form BC, by means of suitable synthetic strategy, and the steps may be performed according to the methods known in the art, e.g. like nucleophilic displacement or reductive amination or use of precursors like nitro functionality, etc. The synthesis may also be carried out in one pot as a multicomponent reaction.

According to a further object, the present invention is also concerned with pharmaceutical compositions comprising a compound of formula (I) together with a pharmaceutically acceptable excipient or carrier.

According to a still further object, the present invention is also concerned with the use of a compound of formula (I) for the preparation of a medicament for the treatment and/or prevention of tissue graft and/or transplant rejection and various auto-immune disorders.

According to a still further object, the present invention is also concerned with the use of a compound of formula (I)

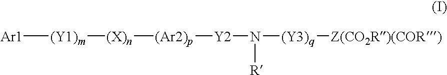

wherein
Ar1 represents an Aryl or Heteroaryl group optionally substituted by 1 up to 5 R group(s),

wherein, each R, identical or different, is selected from the group consisting in Halogen atom, perhalogenoalkyl, -Alkyl, —OAlkyl, —OH, —COOR7, —CONR7R8, -Alkyl-Hal, —OAlkyl-Hal, NO2, —CN, —NR7R8, -AlkylAryl, -Aryl, —S(O)lR7, -Alkenyl, —Si(Alkyl)₃;

wherein R7 and R8, identical or different, are chosen from the group consisting in H; Alkyl; Cycloalkyl; Aryl; -AlkylAryl; Heteroaryl; wherein Alkyl, Cycloalkyl, and/or Aryl is(are) optionally substituted by one or more identical or different Halogen atom, polyfluoroalkyl, Aryl, —COOR7
or R7 and R8 form together with the N atom to which they are attached a Heterocycle;
l is 0, 1 or 2

Y1 represents an -Alkyl-chain, optionally substituted by one or more R as defined above, and optionally comprising one or more unsaturation(s) and/or heteroatom(s) and/or a residue chosen from the group consisting in -Alkenyl-, -Alkynyl-, —CH═N—, —N═CH—, —CH═N—O—, —O—N═CH—, —C(═O)—, —C(═O)(OR7)-, —C(═O)(NR7)-, —N═N—, —S(O)l-;
m is 0 or 1;
X represents a heteroatom;
n is 0 or 1;

—Ar2- represents an -Aryl-group optionally substituted by one or more R group as defined above or wherein 2 R may form together with the atoms to which they are attached a fused cyclic, aryl or heteroaryl ring
p is 0 or 1;
—Y2- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7), —N═N—, —S(O)l—;
—R′ represents a hydrogen atom, or a -Cycloalkyl or an -Alkyl chain, the cycloalkyl or alkyl being optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7)-, —N═N—, —S(O)l-, such as -Alkenyl-Alkyl, -Alkynyl-Alkyl, —CH═N-Alkyl, —N═CH-Alkyl, —CH═N—O-Alkyl, —O—N═CH-Alkyl, —C(═O)-Alkyl, —C(═O)O-Alkyl, —C(═O)(NR7)-Alkyl, —N═N-Alkyl, —S(O)l-Alkyl;
—Y3- represents an -Alkyl-chain, optionally substituted by one or more R as defined above, and optionally comprising one or more unsaturation(s) or heteroatom(s) and/or a residue chosen from the group consisting in -Alken-, -Alkynyl-, —CH═N—, —N═CH—, —CH═N—O—, —O—N═CH—, —C(═O)—, —C(═O)O—, —C(═O)(NR7)-, —N═N—, —S(O)l-;
q is 0 or 1;
Z represents a -Cycloalkyl< group, an -Heterocyclic<, an -Aryl<, -Heteroaryl< group or a —CH<, —C(alkyl)< group, or R′ and Z form together with the N atom to which they are attached a Heterocyclic or Heteroaryl group;
   wherein (CO2R″) and (COR′″) can be attached to the same atom or two adjacent atoms;
R′″ represents a hydrogen atom or an Alkyl chain, optionally substituted by one or more R as defined above, and optionally comprising one or more unsaturation(s);
—R′″ represents —OH, —OAlkyl, H, —NR7OR8, —NR7R8, natural or synthetic amino acids, wherein R7 and R8 are defined as above;

or R″ and R′″ form together a ring with the atom(s) to which they are attached to form a cyclic intramolecular bis carbonyl group, including Meldrum acid derivatives;

as well as their enantiomers, diastereoisomers, geometrical isomers, mixtures thereof, free forms and pharmaceutically acceptable salts, hydrates, solvates and esters

for the preparation of a medicament for decreasing circulating lymphocytes in blood in a human patient in the need thereof.

According to a preferred aspect, such medicament is suitable as immunosuppressive agent. More preferably, such medicament is particularly suitable for the treatment and/or prevention transplant rejection, auto-immune diseases, inflammatory and chronic inflammatory conditions that include rheumatoid arthritis, asthma, pollinosis, psoriasis, myocarditis, atopic dermatitis, lymphocytic leukemias, lymphomas, multiple sclerosis, lupus erythematosus, inflammatory bowel diseases, diabetes mellitus, glomerulonephritis, atherosclerosis, multiorgan failure, sepsis, pneumonia as well as disorders related to impaired vascular integrity, deregulated angiogenesis; more preferably said medicament is for treating and/or preventing tissue graft rejection.

According to a still further object, the present invention is also concerned with the use of a compound of formula (I) for the preparation of a medicament interacting with sphingosine phosphate receptor, preferably acting selectively as agonist of human S1P1 receptor, to be administered to a patient in the need thereof. Preferably, such medicament also acts as an agonist of human S1P2 receptors, and more preferably is substantially inactive at S1P3 receptors.

According to a still further object, the present invention also concerns the methods of treatment comprising administering an effective amount of a compound of the invention for treating and/or preventing the above conditions or disorders The present invention also provides methods for interacting with SP receptors, preferably acting as agonist, preferably interacting selectively with S1P1 receptors, comprising administering an effective amount of a compound of the invention to a patient in the need thereof. Preferably, such methods are also suitable for interacting with S1P2 receptors, and more preferably without any substantial interaction with S1P3 receptors.

The identification of those subjects who are in need of treatment of herein-described diseases and conditions is well within the ability and knowledge of one skilled in the art. A clinician skilled in the art can readily identify, by the use of clinical tests, physical examination and medical/family history, those subjects who are in need of such treatment.

A therapeutically effective amount can be readily determined by the attending diagnostician, as one skilled in the art, by the use of conventional techniques and by observing results obtained under analogous circumstances. In determining the therapeutically effective amount, a number of factors are considered by the attending diagnostician, including, but not limited to: the species of subject; its size, age, and general health; the specific disease involved; the degree of involvement or the severity of the disease; the response of the individual subject; the particular compound administered; the mode of administration; the bioavailability characteristic of the preparation administered; the dose regimen selected; the use of concomitant medication; and other relevant circumstances.

The amount of a compound of formula (I), which is required to achieve the desired biological effect, will vary depending upon a number of factors, including the dosage of the drug to be administered, the chemical characteristics (e.g. hydrophobicity) of the compounds employed, the potency of the compounds, the type of disease, the diseased state of the patient, and the route of administration.

In general terms, the compounds of this invention may be provided in an aqueous physiological buffer solution containing 0.1 to 10% w/v compound for parenteral administration. Typical dose ranges are from 1 μg/kg to 0.1 g/kg of body weight per day; a preferred dose range is from 0.01 mg/kg to 10 mg/kg of body weight per day. A preferred daily dose for adult humans includes 5, 50, 100 and 200 mg, and an equivalent dose in a human child. The preferred dosage of drug to be administered is likely to depend on such variables as the type and extent of progression of the disease or disorder, the overall health status of the particular patient, the relative biological efficacy of the compound selected, and formulation of the compound excipient, and its route of administration.

The compounds of the present invention are capable of being administered in unit dose forms, wherein the term “unit dose” means a single dose which is capable of being administered to a patient, and which can be readily handled and packaged, remaining as a physically and chemically stable unit dose comprising either the active compound itself, or as a pharmaceutically acceptable composition, as described hereinafter. As such, typical daily dose ranges are from 0.01 to 10 mg/kg of body weight. By way of general guidance, unit doses for humans range from 0.1 mg to 1000 mg per day. Preferably the unit dose range is from 1 to 500 mg administered one to four times a day, and even more preferably from 10 mg to 300 mg, two times a day. Compounds provided herein can be formulated into pharmaceutical compositions by admixture with one or more pharmaceutically acceptable excipients. Such compositions may be prepared for use in oral administration, particularly in the form of tablets or capsules; or parenteral administration, particularly in the form of liquid solutions, suspensions or emulsions; or intranasally, particularly in the form of powders, nasal drops, or aerosols; or dermally, for example, topically or via trans-dermal patches or by rectal administration.

The compositions may conveniently be administered in unit dosage form and may be prepared by any of the methods well known in the pharmaceutical art, for example, as described in Remington: The Science and Practice of Pharmacy, 20th ed.; Gennaro, A. R., Ed.; Lippincott Williams & Wilkins: Philadelphia, Pa., 2000. Pharmaceutically compatible binding agents and/or adjuvant materials can be included as part of the composition. Oral compositions will generally include an inert diluent carrier or an edible carrier.

The tablets, pills, powders, capsules, troches and the like can contain one or more of any of the following ingredients, or compounds of a similar nature: a binder such as microcrystalline cellulose, or gum tragacanth; a diluent such as starch or lactose; a disintegrant such as starch and cellulose derivatives; a lubricant such as magnesium stearate; a glidant such as colloidal silicon dioxide; a sweetening agent such as sucrose or saccharin; or a flavoring agent such as peppermint, or methyl salicylate. Capsules can be in the form of a hard capsule or soft capsule, which are generally made from gelatin blends optionally blended with plasticizers, as well as a starch capsule. In addition, dosage unit forms can contain various other materials that modify the physical form of the dosage unit, for example, coatings of sugar, shellac, or enteric agents. Other oral dosage forms syrup or elixir may contain sweetening agents, preservatives, dyes, colorings, and flavorings. In addition, the active compounds may be incorporated into fast dissolve, modified-release or sustained-release preparations and formulations, and wherein such sustained-release formulations are preferably bi-modal.

Preferred formulations include pharmaceutical compositions in which a compound of the present invention is formulated for oral or parenteral administration, or more preferably those in which a compound of the present invention is formulated as a tablet. Preferred tablets contain lactose, cornstarch, magnesium silicate, croscarmellose sodium, povidone, magnesium stearate, or talc in any combination. It is also an aspect of the present disclosure that a compound of the present invention may be incorporated into a food product or a liquid.

Liquid preparations for administration include sterile aqueous or non-aqueous solutions, suspensions, and emulsions. The liquid compositions may also include binders, buffers, preservatives, chelating agents, sweetening, flavoring and coloring agents, and the like. Non-aqueous solvents include alcohols, propylene glycol, polyethylene glycol, acrylate copolymers, vegetable oils such as olive oil, and organic esters such as ethyl oleate. Aqueous carriers include mixtures of alcohols and water, hydrogels, buffered media, and saline. In particular, biocompatible, biodegradable lactide polymer, lactide/glycolide copolymer, or polyoxyethylene-polyoxypropylene copolymers may be useful excipients to control the release of the active compounds. Intravenous vehicles can include fluid and nutrient replenishers, electrolyte replenishers, such as those based on Ringer's dextrose, and the like. Other potentially useful parenteral delivery systems for these active compounds include ethylene-vinyl acetate copolymer particles, osmotic pumps, implantable infusion systems, and liposomes.

Alternative modes of administration include formulations for inhalation, which include such means as dry powder, aerosol, or drops. They may be aqueous solutions containing, for example, polyoxyethylene-9-lauryl ether, glycocholate and deoxycholate, or oily solutions for administration in the form of nasal drops, or as a gel to be applied intranasally. Formulations for buccal administration include, for example, lozenges or pastilles and may also include a flavored base, such as sucrose or acacia, and other excipients such as glycocholate. Formulations suitable for rectal administration are preferably presented as unit-dose suppositories, with a solid based carrier, such as cocoa butter, and may include a salicylate. Formulations for topical application to the skin preferably take the form of an ointment, cream, lotion, paste, gel, spray, aerosol, or oil. Carriers which can be used include petroleum jelly, lanolin, polyethylene glycols, alcohols, or their combinations. Formulations suitable for transdermal administration can be presented as discrete patches and can be lipophilic emulsions or buffered, aqueous solutions, dissolved and/or dispersed in a polymer or an adhesive.

The invention is further illustrated but not restricted by the description in the following examples.

| | Compound | | | | | | |
|---|---|---|---|---|---|---|---|
| | 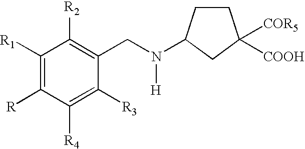 | | | | | | |
| Ex | R | R1 | R2 | R3 | R4 | R5 | MS (ESI+) |
| 1 | H17C8 | H | H | H | H | OH | 376.5 |
| 2 | H17C8 | H | H | H | H | OEt | — |
| 3 | H19C9 | H | H | H | H | OH | 390.4 |
| 4 | H17C8O | H | H | H | H | OH | 392.5 |
| 5 | H17C8O | OMe | H | H | H | OH | — |
| 6 | H9C4O | OMe | H | H | H | OH | 366.4 |
| 7 | H13C6O | OMe | H | H | H | OH | 394.3 |
| 8 | H1708O | Br | H | H | OMe | OH | 502.5 & |
| | | | | | | | 500.4 |
| 9 | H17C8O | OMe | H | H | Me | OH | 436.5 |
| 10 | H17C8O | Cl | H | H | OMe | OH | 457.4 & |
| | | | | | | | 455.5 |
| 11 | H19C9O | H | H | H | H | OH | 406.43 |
| 12 | H21C1O | H | H | H | H | OH | 404.48 |
| 13 | H19C9O | OMe | H | H | H | OH | 436.47 |
| 14 | H19C9O | Cl | H | H | H | OH | 442.5 & |
| | | | | | | | 440.5 |
| 15 | 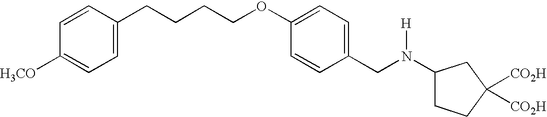 | | | | | | 442.5 |
| 16 | 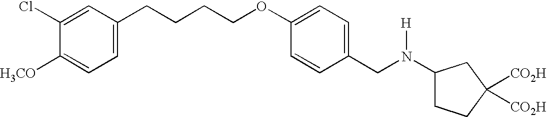 | | | | | | 478.4 & 476.4 |
| 17 | H19C9 | H | H | H | H | OEt | 418.3 |
| 18 | H13C6n-4-Ph | H | H | H | F | OH | 442.3 |
| 19 | H19C9 | H | H | H | H | NHCH2CF3 | 471.3 |
| | | | | | | (Isomer A) | |
| 20 | H19C9 | H | H | H | H | NHCH2CF3 | 471.1 |
| | | | | | | (Isomer B) | |
| 21 | H21C10O | H | H | H | H | OH | 420.3 |
| 22 | H19C9 | H | H | H | H | NHMe | 403.4 |
| | | | | | | (Isomer A) | |
| 23 | H19C9 | H | H | H | H | NHMe | 403.4 |
| | | | | | | (Isomer B) | |
| 24 | 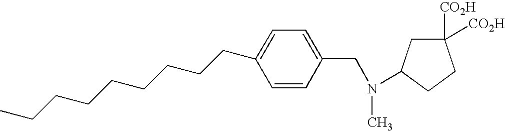 | | | | | | 404.2 |
| 25 | 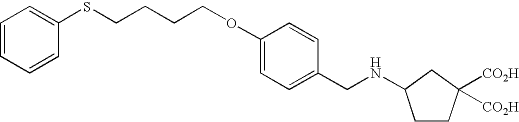 | | | | | | 444.3 |
| 26 | 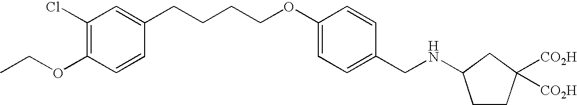 | | | | | | 492.2 & 490.2 |
| | 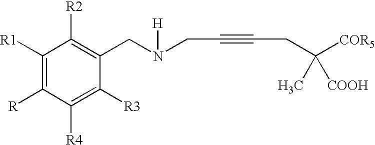 | | | | | | |
| 27 | H19C9 | H | H | H | H | OH | 402.3 |
| 28 | H19C9O | Cl | H | H | H | OH | 454.2 & |
| | | | | | | | 452.3 |
| 29 | H17C8 | H | H | H | H | OH | 388.3 |
| 30 | H21C10 | H | H | H | H | OH | 416.3 |
| 31 | H13C6n-4-Ph | H | H | H | F | OH | 454.3 |

### General Experiment Procedure

### A—Preparation of 3-oxocyclopentane-1,1-dicarboxylic acid diethyl ester

### Step I

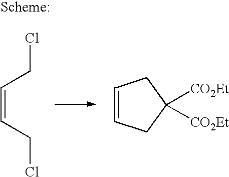

A solution of diethyl malonate (34.59 g, 0.216 mole) in THF (100 mL) is added to a solution of sodium hydride (60% w/w, 21.6 g, 0.54 mole) in THF (200 mL). The mixture is heated under reflux for 1 h, cis-1,4-dichlorobut-2-ene (27 g, 0.216 mole) in THF (100 mL) is added dropwise to the reaction mixture at 750 C. over a period of 15 min. The reaction mixture is heated under reflux for 5 h, cooled to room temperature and quenched with water (50 mL). After removal of solvent the residue obtained is extracted with ethyl acetate (2×100 mL), combined organic extracts are washed with brine and dried over anhydrous sodium sulphate. The crude product obtained upon solvent removal under reduced pressure is purified by column chromatography (silica gel 230-400 mesh, ethyl acetate:hexane 10:90) to get diethyl ester of cyclopent-3-ene-1,1-dicarboxylic acid.

### Step II

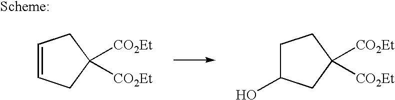

A solution of cyclopent-3-ene-1,1-dicarboxylic acid diethyl ester (15 g, 0.0706 mole) in THF (40 mL) is added slowly to mercuric acetate (24.77 g, 0.078 mole) in water-THF (110 mL, 7:4) at room temperature and stirred for 10 minutes. Perchloric acid (0.5 mL) is added to the reaction mixture (yellow ppt. disappears) continued stirring for 30 minutes. The reaction mixture is cooled to ˜50 C and a solution of sodium hydroxide (4.24 g, in 32 mL water) and sodium borohydride (4.27 g, 0.112 mole) is added. The reaction mixture is stirred for 1.25 h and filtered through celite. The organic layer is separated from the filtrate and the aqueous layer is extracted with ethyl acetate (2×30 mL). Combined organic layers are washed with brine and dried over anhydrous sodium sulphate. Solvent is removed under reduced pressure and the residue is purified by column chromatography (silica gel 230-400 mesh, n-hexane:ethyl acetate 1:1) to get 3-hydroxy-cyclopentane-1,1-dicarboxylic acid diethyl ester.

### Step III

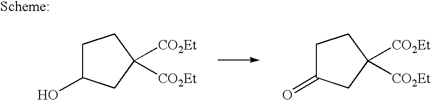

Jone's reagent (11.84 mL) is added dropwise to a solution of 3-hydroxy-cyclopentane-1,1-dicarboxylic acid diethyl ester (1.8 g, 0.0078 mole) in acetone (20 mL) at 10-150 C. The mixture is stirred at room temperature for 1 h, further quantity of the reagent (11.84 mL) is added and stirred for 2 more hours. The reaction is treated with isopropanol (5 mL) and concentrated under reduced pressure. Water is added to the residue and extracted with ethyl acetate (2×30 mL). Combined organic extracts are washed with brine and dried over anhydrous sodium sulphate. Removal of solvent under reduced pressure yields diethyl ester of 3-oxo-cyclopentane-1,1-dicarboxylic acid.

### B—Preparation of 3-bromo-5-methoxy-4-octyloxybenzylamine

### Step-I

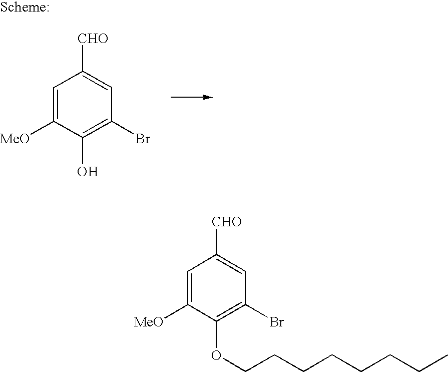

1-Iodooctane (9.4 mL, 0.051 mole) is added to a solution of 5-bromovanillin (10.0 g, 0.043 mole) in DMF (50 mL) at room temperature. Potassium carbonate (9.55 g, 0.069 mole) is added and the reaction mixture is heated at 1200 C. for 1.5 h. Reaction mixture is concentrated under vacuum, water (50 mL) is added to the residue and extracted with ethyl acetate (3×30 mL). The combined organic layers are washed with water (1×20 mL) followed by brine solution (1×25 mL) and dried over Na2SO4. Removal of ethyl acetate gives light brown viscous liquid which is purified by column chromatography (silica gel 230-400 mesh, n-hexane-ethyl acetate, 92:8) to get 3-bromo-5-methoxy-4-octyloxybenzaldehyde.

### Step-II

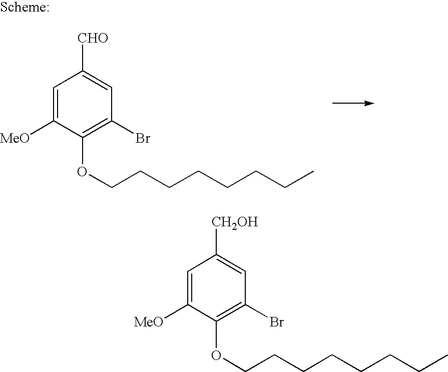

Sodium borohydride (0.27 g, 0.0073 mole) is added in portions to a stirred THF (30 mL) solution of 3-bromo-5-methoxy-4-octyloxybenzaldehyde (5 g, 0.0146 mole) at room temperature and stirred for 45 min. The reaction mixture is concentrated under vacuum, water (20 mL) is added to the residue and extracted with dichloromethane (3×25 mL). The combined organic layers are washed with water (1×15 mL) followed by brine solution (1×15 mL) and dried over anhydrous Na2SO4. Removal of solvent furnishes 3-bromo-5-methoxy-4-octyloxyphenylmethanol.

### Step-III

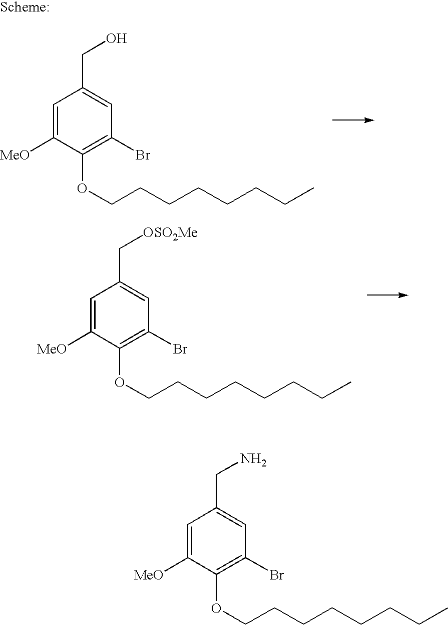

Triethylamine (8.0 mL, 0.0578 mole) is added to a dichloromethane solution (40 mL) of 3-bromo-5-methoxy-4-octyloxybenzylalcohol (4.98 g, 0.0144 mole). Reaction mixture is cooled to 00 to −50 C and methanesulphonyl chloride (3.4 mL, 0.0433 mole) is added dropwise and stirred for 10 minutes at 00-50 C, followed by stirring at room temperature for 3.5 h. Reaction mixture is quenched with water (15 mL) under ice cold condition and aqueous layer is extracted with dichloromethane (3×25 mL). The combined dichloromethane layers are washed with water (1×20 mL) followed by brine solution (1×20 mL) and dried over anhydrous Na2SO4. Removal of dichloromethane furnishes crude 3-bromo-5-methoxy-4-octyloxybenzyl mesylate.

Crude 3-bromo-5-methoxy-4-octyloxybenzyl mesylate is dissolved in methanol (10 mL) and cooled to 00 to −50 C. Methanolic ammonia (˜100 mL) is added to the cooled reaction mixture and stirred for 10 min, followed by stirring at room temperature for 18 h. Excess ammonia is removed and reaction mixture is concentrated under vacuum. The crude material is purified by column chromatography (silica gel 230-400 mesh, methanol: ethyl acetate; 20:80) to obtain the desired 3-bromo-5-methoxy-4-octyloxybenzylamine.

The following benzylamines were also prepared using procedure analogous to the one described above:

| | Compound | | | | |
|---|---|---|---|---|---|
| | 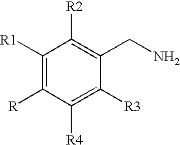 | | | | |
| Example | R | R1 | R2 | R3 | R4 |
| 4 | H17C8O | H | H | H | H |
| 5 | H17C8O | OMe | H | H | H |
| 6 | H9C4O | OMe | H | H | H |
| 7 | H13C6O | OMe | H | H | H |
| 9 | H17C8O | OMe | H | H | Me |
| 10 | H17C8O | Cl | H | H | OMe |
| 11 | H19C9O | H | H | H | H |
| 13 | H19C9O | OMe | H | H | H |
| 14 | H19C9O | Cl | H | H | H |

| | |
|---|---|
| 15 | 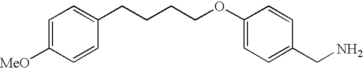 |
| 16 | 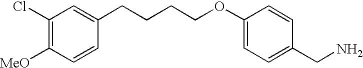 |

### C—Preparation of 4-nonylbenzylamine

### Step I

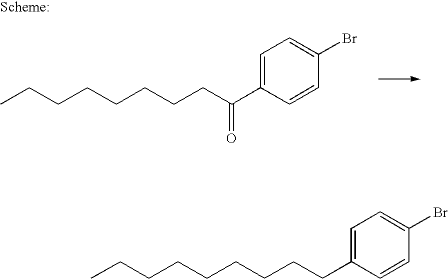

Triethylsilane (20 mL) is added dropwise to a solution of 1-(4-bromophenyl)nonan-1-one (10 g, 0.033 mole) in TFA (20 mL) stirred at room temperature. The mixture is then heated under reflux for 4.5 h. The reaction mixture is cooled and concentrated under reduced pressure. The resulting residue is purified by column chromatography (silica gel 230-400 mesh, n-hexane:ethyl acetate 95:5) to get 1-bromo-4-nonylbenzene.

Ref: J. Org. Chem. 38, 2675 (1973)

### Step II

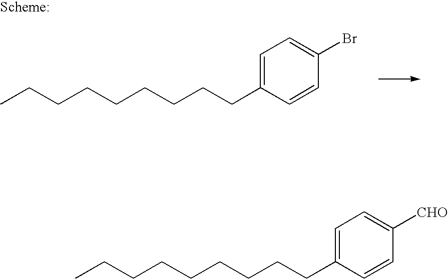

To a heterogeneous solution of magnesium (0.771 g, 0.032 mole) in THF ((10 mL) under nitrogen atmosphere are added few crystals of iodine, followed by 5 mL of 1-bromo-4-nonylbenzene (6.5 g, 0.0229 mole) in THF (40 mL) with vigorous stirring at 65° C. After initiation, rest of the solution of 1-bromo-4-nonylbenzene is added dropwise to the reaction mixture and heated under reflux for 1 h. The reaction mixture is cooled to 0 to −50 C, a solution of THF and DMF (15 mL+3.5 mL) is added and stirred at room temperature for 3 h. The reaction is quenched with 6N HCl (50 mL) at 0° to −50° C. and extracted with ethyl acetate (2×40 mL). Combined organic layers are washed with brine (1×15 mL) and dried over Na2SO4. Removal of solvent gives crude viscous liquid which is purified by column chromatography (silica gel 230-400 mesh, n-hexane:ethyl acetate 95:5) to get 4-nonyl-benzaldehyde.

### Step III

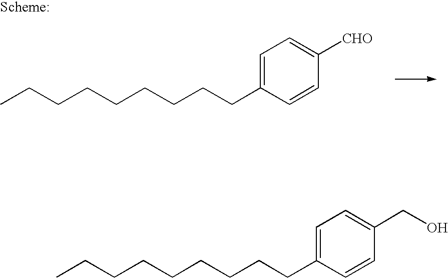

Sodium borohydride (0.847 g. 0.022 mole) is added portionwise to a solution of 4-nonylbenzaldehyde (2.6 g, 0.011 mole) in methanol (30 mL) over a period of 15 min at room temperature and further allowed to stir for 2 h. The reaction is concentrated under reduced pressure, quenched with water (10 mL) and extracted with ethyl acetate (3×30 mL). Combined organic extracts are washed with brine (1×15 mL) and dried (Na2SO4). Removal of solvent furnishes 4-nonyl-phenylmethanol.

### Step IV

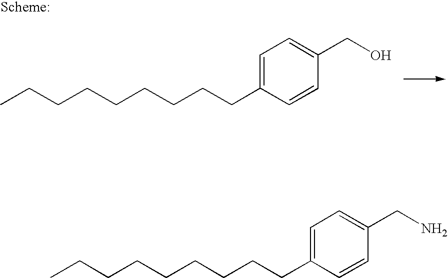

Methane sulphonyl chloride (1.9 g., 0.016 mole) is added drop wise to a solution of 4-nonylphenylmethanol (2.6 g., 0.011 mole) and triethyl amine (2.24 g., 0.022 mole) in MDC (30 ml) at 0 to 50 C. The reaction mixture is allowed to stir at room temperature for 2 hours, quenched with water (20 ml) and extracted with MDC (2×25 ml). Combined organic extract is washed with brine (1×15 ml) and dried (Na2SO4). Removal of solvent furnished crude mesylated product.

To this mesylated product methanolic ammonia (50 ml) is added and allowed to stir at room temperature for overnight. Solvent is removed under vacuum and the residue is purified by column chromatography (silica gel 230-400 mesh, methanol:ethyl acetate 30:70) to get 4-nonylbenzylamine.

The following benzyl amines were also prepared using procedure analogous to the described above:

| | Compound | | | | |
|---|---|---|---|---|---|
| | 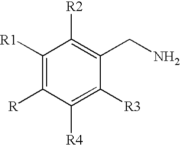 | | | | |
| Example | R | R1 | R2 | R3 | R4 |
| 1 | H17C8 | H | H | H | H |
| 12 | H21C10 | H | H | H | H |

### D. Preparation of 3-oxocyclopentane-1,1-dicarboxylic acid ethyl ester

### Step I

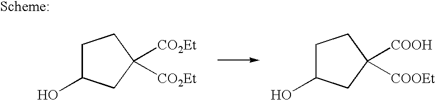

To a stirred solution of 3-hydroxycyclopentane-1,1-dicarboxylic acid diethyl ester (0.85 g, 0.0037 mole) in ethanol (7 mL), a solution of sodium hydroxide (0.162 g, 0.0040 mole) in water (3 mL) is added at room temperature. The reaction mixture is heated under reflux for 1.5 h. It is then concentrated under reduced pressure and the residue is quenched with water (10 mL). The aqueous layer after washing with ethyl acetate (1×10 mL) is acidified to pH ˜2 by dropwise addition of dil. HCl. It is then extracted with ethyl acetate (3×15 mL). Combined organic extracts are washed with brine and dried over sodium sulphate. Removal of solvent furnishes 3-hydroxycyclopentane-1,1-dicarboxylic acid ethyl ester.

### Step II

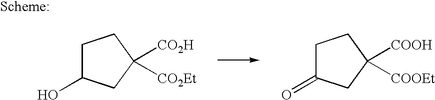

Jone's Reagent (11.88 mL) is added dropwise to a solution of 3-hydroxy-cyclopentane-1,1-dicarboxylic acid ethyl ester (0.8 g, 0.0040 mole) in acetone (10 mL) at 10-150 C. The mixture is stirred at room temperature for 4 hours. The reaction mixture is treated with isopropanol (10 mL) and concentrated under reduced pressure. Water is added to the residue and extracted with ethyl acetate (3×10 mL). Combined organic extracts are washed with brine and dried over anhydrous sodium sulphate. Removal of solvent under reduced pressure yields 3-oxo-cyclopentane-1,1-dicarboxylic acid ethyl ester.

### E. Preparation of 3-oxo-1-(2,2,2-trifluoroethylcarbamoyl)cyclopentanecarboxylic acid ethyl ester

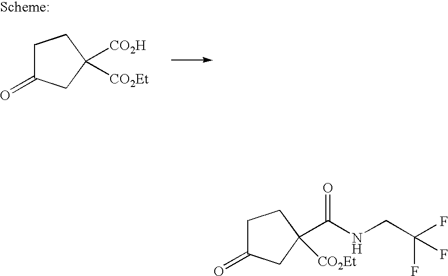

Isobutyl chloroformate (0.49 mL, 0.00378 mole) is added to a solution of 3-oxo-cyclopentane-1,1-dicarboxylic acid ethyl ester (0.75 g, 0.00375 mole) and N-methyl-morpholine (0.62 mL., 0.0056 mole) in tetrahydrofuran (10 mL) at −10 to −150° C. temperature. Stir the reaction mixture at −10° to −150° C. for 1 h, triethyl amine (0.78 mL, 0.0056 mole) and 2,2,2-trifluoroethylamine hydrochloride (0.51 g, 0.00377 mole) are added. The reaction mixture is slowly brought to room temperature and stirred at room temperature for 1.5 h. It is then quenched with water (5 mL) and extracted with ethyl acetate (2×15 mL). Combined organic extracts are washed with saturated sodium bicarbonate solution and dried over anhydrous sodium sulphate. Removal of solvent under reduced pressure yields a viscous liquid which is purified by column chromatography (silica gel 230-400 mesh, hexane:ethyl acetate 1:1) to get 3-oxo-1-(2,2,2-trifluoroethylcarbamoyl)-cyclopentanecarboxylic acid ethyl ester.

### F. Preparation of 3-[methyl-(4-nonylbenzyl)amino]-cyclopentane-1,1-dicarboxylic acid diethyl ester

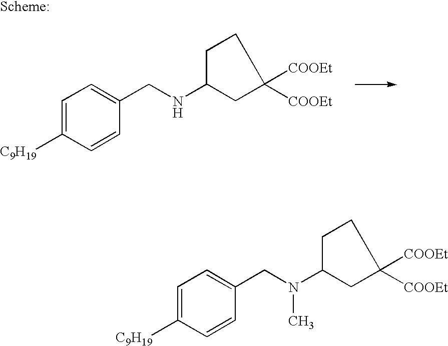

To a stirred solution of 3-(4-nonylbenzylamino)cyclopentane-1,1-dicarboxylic acid ethyl ester (0.6 g, 0.0013 mole) in acetone (15 mL) is added potassium carbonate (0.24 g, 0.0017 mole). The reaction mixture is cooled to 5-100 C and methyl iodide (0.075 mL, 0.0012 mole) is added. The reaction mixture is brought to room temperature and stirred for 4 hours. It is then concentrated under reduced pressure, quenched with water (5 ml) and extracted with ethyl acetate (2×5 mL). Combined organic layers are washed with brine (1×5 mL). After drying over sodium sulphate removal of solvent gives a viscous liquid which is purified by column chromatography (silica gel 230-400 mesh, ethyl acetate:hexane 1:4) to get 3-[methyl-(4-nonylbenzyl)amino]cyclopentane-1,1-dicarboxylic acid diethyl ester.

### G. Preparation of 2-(4-aminobut-2-ynyl)-2-methylmalonic acid diethyl ester

### Step-I

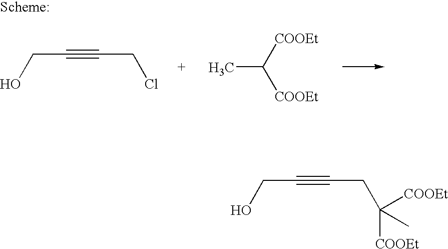

Potassium-tert-butoxide (6.17 g, 0.055 mole) is added to a stirred solution of diethyl methylmalonate (11.0 mL, 0.064 mole) in N,N-dimethylformamide (40 mL) at room temperature. The reaction mixture is heated at 900 C for one hour, cooled to room temperature and a solution of 4-chloro-but-2-yn-1-ol (4.80 g, 0.045 mole) in N,N-dimethylformamide (10 mL) is added and stirred for 2 hours at room temperature. Reaction mixture is quenched with water, concentrated under vacuum and aqueous layer is extracted with ethyl acetate (3×25 mL). The combined organic layers are washed with water (1×30 mL) followed by brine solution (1×20 mL) and dried over anhydrous Na2SO4. Removal of ethyl acetate gives colourless viscous liquid which is purified by column chromatography (n-hexane:ethyl acetate, 75:25) to get 2-(4-hydroxybut-2-ynyl)-2-methylmalonic acid diethyl ester.

### Step-II

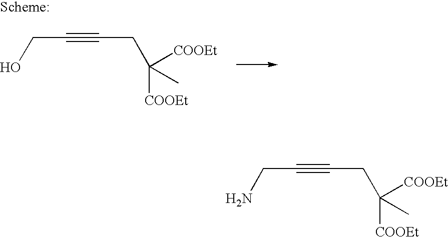

Triethylamine (3.86 mL, 0.027 mole) is added to a solution of 2-(4-hydroxybut-2-ynyl)-2-methyl malonic acid diethyl ester (4.20 g, 0.0173 mole) in dichloromethane. Methanesulphonyl chloride (1.61 mL, 0.02 mole) is added dropwise to the ice cooled (00-50 C) reaction mixture followed by stirring at room temperature for 30 minutes. Reaction mixture is quenched with water under ice cold condition and aqueous layer is extracted with dichloromethane. The combined dichloromethane layers are washed with water (1×15 mL) followed by brine (1×15 mL) and finally dried over anhydrous sodium sulphate. Removal of dichloromethane gives mesylate derivate as a viscous liquid.

Mesylated derivative is dissolved in methanol and methanolic ammonia is added under ice cold condition. Reaction mixture is stirred for 13 h at room temperature. Excess ammonia is removed and reaction mixture is concentrated under vacuum. The crude material is purified by passing through silica gel column (ethyl acetate:methanol, 80:20) which gives 2-(4-aminobut-2-ynyl)-2-methylmalonic acid diethyl ester.

### EXAMPLES 1 AND 2

### 3-(4-Octylbenzylamino)cyclopentane-1,1-dicarboxylic acid hydrochloride and 3-(4-octylbenzylamino)cyclopentane-1,1-dicarboxylic acid ethyl ester

To a stirred solution of 4-octylbenzylamine (0.53 g, 0.00242 mole) in dichloromethane (20 mL) and methanol (2 mL), a solution of 3-oxocyclopentane-1-1,1-dicarboxylic acid diethyl ester (0.55 g, 0.00241 mole) in dichloromethane (5 mL) is added at room temperature. The reaction mixture is cooled to 5-100 C and sodium cyanoborohydride (0.530 g, 0.0084 mole) is added. The reaction mixture is brought to room temperature and is allowed to stir at room temperature for 4 h. The reaction mixture is concentrated under reduced pressure and the residue is quenched with water (10 mL). It is extracted in ethyl acetate (2×20 mL). Combined organic layers are washed with brine (1×10 mL) and dried over sodium sulphate. Removal of solvent gives a viscous liquid which is purified by column chromatography (silica gel 230-400 mesh, mobile phase n-hexane:ethyl acetate 7:3) to get 3-(4-octylbenzylamino)cyclopentane-1,1-dicarboxylic acid diethyl ester as a colourless viscous liquid.

To a stirred solution of 3-(4-octylbenzylamino)cyclopentane-1,1-dicarboxylic acid diethyl ester (0.8 g, 0.0018 mole) in ethanol (8 mL), a solution of sodium hydroxide (0.081 g, 0.0020 mole) in water (8 mL) is added at room temperature.

The reaction mixture is heated under reflux for 1 h. The reaction mixture is concentrated under reduced pressure and the residue is quenched with water (7 mL). The aqueous solution is neutralised to pH ˜6 by dropwise addition of dil. HCl.

It is then concentrated under reduced pressure. The residue is purified by column chromatography (silica gel 230-400 mesh, mobile phase methanol:ethyl acetate 30:70) to get 3-(4-octylbenzylamino)cyclopentane-1,1-dicarboxylic acid ethyl ester as a white solid.

To a stirred solution of 3-(4-octylbenzylamino)cyclopentane-1,1-dicarboxylic acid ethyl ester (0.6 g, 0.0014 mole) in ethanol (6 mL), a solution of sodium hydroxide (0.17 g, 0.0044 mole) in water (6 mL) is added at room temperature. The reaction mixture is heated under reflux for 5 h. The reaction mixture is concentrated under reduced pressure and the residue is quenched with water (2 mL). The aqueous solution is acidified to pH ˜2 by dropwise addition of dil. HCl. It is then extracted with tetrahydrofuran (2×10 mL). Combined organic layers are washed with brine (1×10 mL) and dried over anhydrous sodium sulphate. Removal of solvent under reduced pressure gives sticky solid (0.2 g.) which is washed with ether (2×10 mL) to get 3-(4-octylbenzylamino)cyclopentane-1,1-dicarboxylic acid HCl salt.

13C NMR: (TFA+CDCl3; 100.61 MHz; ppm)

14.68 (q); 23.30 (t); 29.39 (t); 29.88 (t); 30.00 (t); 30.05 (t); 31.87 (t); 32.52 (t); 33.41 (t); 36.29 (t); 37.43 (t); 51.60 (t); 58.36 (d); 59.56 (s); 126.98 (s); 130.16 (2d);

130.38 (2d); 146.29 (s); 175.20 (s); 176.22 (s)

Following examples were prepared and characterised by their 13C data in the free form or as salts in a similar way.

Compound

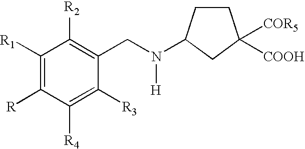

| Ex. | R | R1 | R2 | R3 | R4 | R5 | MS (ESI+) |
|---|---|---|---|---|---|---|---|
| 1 | H17C8 | H | H | H | H | OH | 376.5 |
| 2 | H17C8 | H | H | H | H | OEt | — |
| 3 | H19C9 | H | H | H | H | OH | 390.4 |
| 4 | H17C8O | H | H | H | H | OH | 392.5 |
| 5 | H1708O | OMe | H | H | H | OH | — |
| 6 | H9C4O | OMe | H | H | H | OH | 366.4 |
| 7 | H13C6O | OMe | H | H | H | OH | 394.3 |
| 8 | H17C8O | Br | H | H | OMe | OH | 502.5 & |
| | | | | | | | 500.4 |
| 9 | H17C8O | OMe | H | H | Me | OH | 436.5 |
| 10 | H17C8O | Cl | H | H | OMe | OH | 457.4 & |
| | | | | | | | 455.5 |
| 11 | H19C9O | H | H | H | H | OH | 406.43 |
| 12 | H21C10 | H | H | H | H | OH | 404.48 |
| 13 | H19C9O | OMe | H | H | H | OH | 436.47 |
| 14 | H19C9O | Cl | H | H | H | OH | 442.5 & |
| | | | | | | | 440.5 |

| | | |
|---|---|---|
| 15 | 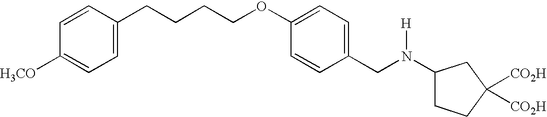 | 442.5 |
| 16 | 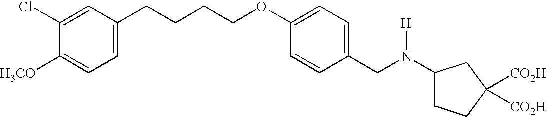 | 478.4 & 476.4 |

### EXAMPLE 3 AS HCL

13C NMR: (CDCl3+TFA; 100.61 MHz; ppm)

14.66 (q); 23.32 (t); 29.48 (t); 29.97 (t); 29.98 (t); 30.10 (t); 30.18 (t); 31.86 (t); 32.55 (t); 33.59 (t); 36.29 (t); 37.40 (t); 51.77 (t); 58.54 (d); 59.82 (s); 126.78 (s); 130.19 (2d); 130.28 (2d); 146.54 (s); 175.62 (s); 176.89 (s)

### EXAMPLE 4

### As HCl

13C NMR: (CDCl3+TFA; 100.61 MHz; ppm)

14.66 (q); 23.28 (t); 26.57 (t); 29.28 (t); 29.75 (t); 29.86 (t); 29.99 (t); 32.45 (t); 33.15 (t); 37.44 (t); 51.28 (t); 58.17 (d); 59.51 (s); 69.06 (t); 115.95 (2d); 121.81 (s); 132.17 (2d); 160.97 (s); 174.99 (s); 175.72 (s)

### EXAMPLE 5

### In the Free Form

13C NMR: (CDCl3+TFA; 50.33 MHz; ppm)

14.54 (q); 23.25 (t); 26.42 (t); 29.43 (t); 29.57 (t); 29.80 (t); 29.91 (t); 32.42 (t); 33.55 (t); 37.39 (t); 51.96 (t); 56.72 (q); 58.75 (d); 59.60 (s); 70.25 (t); 113.95 (2d); 122.18 (d); 124.08 (s); 149.92 (s); 150.53 (s); 175.67 (s); 176.87 (s).

### EXAMPLE 6

### As HCl

13C NMR: (CDCl3+TFA; 100.61 MHz; ppm)

14.25 (q); 19.66 (t); 29.47 (t); 31.48 (t); 33.34 (t); 37.45 (t); 51.73 (t); 56.88 (q); 58.30 (d); 59.50 (s); 69.76 (t); 113.65 (d); 114.23 (d); 122.15 (d); 124.15 (s); 149.86 (s); 150.42 (s); 175.35 (s); 176.12 (s)

### EXAMPLE 7

### In the Free Form

13C NMR: (CDCl3+TFA; 50.33 MHz; ppm)

14.45 (q); 23.12 (t); 26.08 (t); 29.45 (2t); 32.10 (t); 33.38 (t); 37.27 (t); 51.67 (t); 56.59 (q); 58.51 (d); 59.59 (s); 70.01 (t); 113.80 (2d); 122.34 (d); 123.80 (s); 150.06 (s); 150.47 (s); 175.20 (s); 176.51 (s)

### EXAMPLE 8

### As HCl

13C NMR: (CDCl3+TFA; 100.61 MHz, ppm)

14.61 (q); 23.26 (t); 26.37 (t); 29.35 (t); 29.86 (t); 29.94 (t); 30.56 (t); 32.45 (t); 33.37 (t); 37.23 (t); 51.04 (t); 56.56 (q); 58.91 (d); 59.55 (s); 74.98 (t); 113.46 (d); 119.18 (s); 126.72 (s); 127.14 (d); 147.47 (s); 155.03 (s); 174.96 (s); 176.37 (s).

### EXAMPLE 9

### In the Free Form

13C NMR: (Pyridine d5+Methanol d4; 100.61 MHz; ppm)

14.89 (q); 16.93 (q); 23.75 (t); 27.26 (t); 29.31 (t); 30.43 (t); 30.55 (t); 31.37 (t); 31.60 (t); 32.95 (t); 45.31 (t); 45.83 (t); 56.68 (q); 59.48 (d); 59.91 (s); 73.65 (t); 111.56 (d); 123.53 (d); 132.97 (s); 133.22 (s); 147.57 (s); 154.37 (s); 178.22 (s); 179.13 (s)

### EXAMPLE 10

### In the Free Form

13C NMR: (Pyridine d5+Methanol d4; 100.61 MHz; ppm)

15.09 (q); 23.94 (t); 27.31 (t); 29.53 (t); 30.60 (t); 30.67 (t); 31.58 (2t); 33.13 (t); 45.48 (t); 45.63 (t); 57.28 (q); 59.90 (d); 60.01 (s); 74.68 (t); 112.74 (d); 122.29 (d); 129.66 (s); 134.71 (s); 145.78 (s); 155.81 (s); 178.20 (s): 179.65 (s)

### EXAMPLE 11

### As HCl

13C NMR: (CDCl3+TFA; 100.61 MHz; ppm)

14.67 (q); 23.31 (t); 26.55 (t); 29.42 (t); 29.71 (t); 29.90 (t); 30.01 (t); 30.16 (t); 32.52 (t); 33.53 (t); 37.34 (t); 51.51 (t); 58.31 (d); 59.59 (s); 69.16 (t); 116.22 (2d); 121.36 (s); 131.73 (2d); 161.23 (s); 175.31 (s); 176.61 (s)

### EXAMPLE 12

13C NMR: (CDCl3+TFA; 100.61 MHz; ppm)

14.75 (q); 23.35 (t); 29.22 (t); 30.02 (t); 30.12 (t); 30.17 (t); 30.30 (t); 30.31 (t); 31.96 (t); 32.58 (t); 33.37 (t); 36.33 (t); 37.44 (t); 51.11 (t); 58.31 (d); 59.59 (s); 127.73 (s); 129.87 (2d); 130.50 (2d); 145.57 (s); 175.08 (s); 176.15 (s)

### EXAMPLE 13

### As HCl

13C NMR: (CDCl3+TFA; 100.61 MHz; ppm)

14.65 (q); 23.29 (t); 26.48 (t); 29.25 (t); 29.56 (t); 29.89 (t); 30.01 (t); 30.13 (t); 32.49 (t); 33.13 (t); 37.40 (t); 51.60 (t); 56.86 (q); 58.30 (d); 59.50 (s); 69.98 (t); 113.52 (d); 114.36 (d); 122.47 (s); 124.11 (d); 149.85 (s); 150.30 (s); 174.98 (s); 175.70 (s)

### EXAMPLE 14

### As HCl

13C NMR: (CDCl3+TFA); 100.61 MHz; ppm)

14.71 (q); 23.32 (t); 26.53 (t); 29.29 (t); 29.62 (t); 29.91 (t); 29.99 (t); 30.15 (t); 32.53 (t); 33.37 (t); 37.27 (t); 50.60 (t); 58.45 (d); 59.62 (s); 70.05 (t); 114.24 (d); 122.74 (s); 124.14 (s); 130.21 (d); 132.09 (d); 156.59 (s); 175.06 (s); 176.32 (s).

### EXAMPLE 15

### As HCl

13C NMR: (CDCl3+TFA+CD3OD; 100.61 MHz, ppm)

28.48 (t); 29.02 (t); 29.14 (t); 32.53 (t); 35.07 (t); 37.11 (t); 50.47 (t); 55.64 (q); 57.54 (d); 59.57 (s); 68.47 (t); 114.27 (2d); 115.64 (2d); 122.62 (s); 129.80 (2d); 131.73 (2d); 134.91 (s) 158.22 (s); 160.66 (s); 174.19 (s); 175.06 (s).

### EXAMPLE 16

### As HCl

13C NMR: (CDCl3+TFA; 100.61 MHz, ppm)

28.38 (t); 29.25 (t); 29.78 (t); 34.24 (t); 34.97 (t); 37.58 (t); 50.05 (t); 56.79 (q); 58.46 (d); 59.28 (s); 68.38 (t); 112.75 (2d); 115.66 (d); 122.64 (s); 128.22 (2d); 130.65 (2d); 132.06 (s); 135.97 (s); 153.76 (s); 160.58 (s); 175.79 (s); 177.44 (s).

### EXAMPLE 17

### 3-(4-nonylbenzylamino)cyclopentane-1,1-dicarboxylic acid ethyl ester

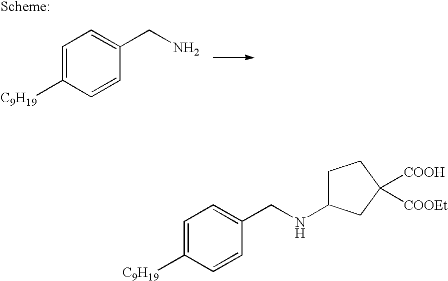

To a stirred solution of 4-nonylbenzylamine (0.2 g, 0.00086 mole) in methanol (5 mL), 3-oxocyclopentane-1,1-dicarboxylic acid ethyl ester (0.188 g, 0.00094 mole) and Triton-B (0.53 mL, 0.0013 mole) are added at room temperature. The reaction mixture is stirred for 5 min, sodium cyanoborohydride (0.08 g, 0.0013 mole) is added and it is heated under reflux for 4 h. The reaction mixture is concentrated under reduced pressure and the residue is quenched with water (5 mL). The aqueous solution is neutralised to pH ˜6 by dropwise addition of dilute hydrochloric acid and extracted with tetrahydrofuran (3×10 mL). Combined organic layers are washed with brine (1×10 mL) and dried over sodium sulphate. Removal of solvent gives a viscous liquid which is purified by column chromatography (silica gel 230-400 mesh, methanol:dichloromethanel:9) to get a white solid. This is converted into the corresponding hydrochloride salt by treatment with ethanolic HCl to get 3-(4-nonylbenzylamino)cyclopentane-1,1-dicarboxylic acid ethyl ester hydrochloride.

13C NMR: (CDCl3; 100.61 MHz; ppm)

14.76 (q); 14.85 (q); 23.33 (t); 29.98 (t); ˜30.0 (t, probably merged); 30.05 (t); 30.15 (t); 30.21 (t); 32.01 (t); 32.54 (t); 33.69 (t); 36.34 (t); 37.57 (t); 48.75 (t); 57.56 (d); 61.50 (t); 62.62 (s); 129.59 (2d); 129.72 (s); 130.41 (2d); 144.39 (s); 173.72 (s); 179.22 (s).

The following further compounds were prepared according to the above procedures:

| | Compound | | | | | | |
|---|---|---|---|---|---|---|---|
| | 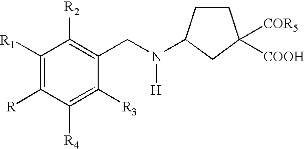 | | | | | | |
| Ex. | R | R1 | R2 | R3 | R4 | R5 | MS (ESI+) |
| 17 | H19C9 | H | H | H | H | OEt | 418.3 |
| 18 | H13C6n-4-Ph | H | H | H | F | OH | 442.3 |
| 19 | H19C9 | H | H | H | H | NHCH2CF3 | 471.3 |
| | | | | | | (Isomer A) | |
| 20 | H19C9 | H | H | H | H | NHCH2CF3 | 471.1 |
| | | | | | | (Isomer B) | |
| 21 | H21C10O | H | H | H | H | OH | 420.3 |
| 22 | H19C9 | H | H | H | H | NHMe | 403.4 |
| | | | | | | (Isomer A) | |
| 23 | H19C9 | H | H | H | H | NHMe | 403.4 |
| | | | | | | (Isomer B) | |

| | | |
|---|---|---|
| 24 | 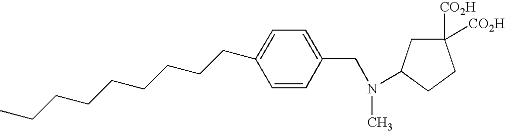 | 404.2 |
| 25 | 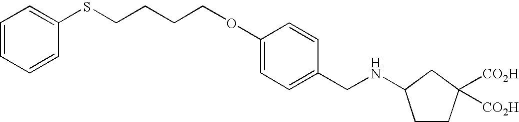 | 444.3 |
| 26 | 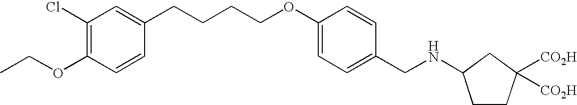 | 492.2 & 490.2 |

### EXAMPLE 18

### As HCl

13C NMR: (CDCl3+TFA; 100.61 MHz; ppm)

14.69 (q); 23.25 (t); 29.19 (t); 29.73 (t); 31.97 (t); 32.35 (t); 33.17 (t); 36.32 (t); 37.20 (t); 50.62 (t); 58.66 (d); 59.71 (s); 117.97 (d, JC—F=24.50 Hz); 126.32 (d); 129.30 (2d); 129.35 (2d); 130.73 (s, JC—F=7.85 Hz); 131.51 (s, JC—F=13.29 Hz); 132.29 (d); 144.06 (s); 160.35 (s, JC—F=249.91 Hz); 174.94 (s); 176.15 (s); One singlet is merged with peak having 6 between 129 and 133.

### EXAMPLE 19

### As HCl

13C NMR: (TFA+CDCl3; 100.61 MHz; ppm)

14.70 (q); 23.32 (t); 29.35 (t); 29.97 (t), 30.05 (t); 30.11 (t); 30.19 (t); 31.90 (t); 32.54 (t); 34.84 (t); 36.30 (t); 37.37 (t); 41.92 [t (q), JCF=33.51 Hz]; 51.35 (t); 57.71 (d); 60.17 (s); 124.50 [s (q), JCF=278.81 Hz,]; 127.07 (s); 130.08 (2d); 130.70 (2d); 146.09 (s); 172.62 (s); 175.75 (s).

### EXAMPLE 20

### As HCl

13C NMR: (TFA+CDCl3; 100.61 MHz; ppm)

14.65 (q); 23.30 (t); 29.96 (t); 29.99 (t), 30.09 (t); 30.12 (t); 30.17 (t); 31.87 (t); 32.53 (t); 36.29 (t); 36.87 (t); 37.67 (t); 41.82 [t (q), JCF=35.19 Hz]; 51.11 (t); 59.34 (d); 59.54 (s); 124.34 [s (q), JCF=278.38 Hz]; 127.32 (s): 130.15 (2d); 130.21 (2d); 146.25 (s); 175.16 (s); 175.26 (s)

### EXAMPLE 21

### As HCl

13C NMR: (TFA+CDCl3; 100.61 MHz; ppm)

14.67 (q); 23.32 (t); 26.57 (t); 29.25 (t); 29.75 (t); 29.97 (t); 30.04 (t); 30.21 (2t); 32.55 (t); 33.22 (t); 37.24 (t); 51.23 (t); 58.16 (d); 59.69 (s); 69.05 (t): 116.05 (2d); 121.72 (s); 131.73 (2d); 161.09 (s); 174.89 (s); 176.23 (s).

### EXAMPLE 22

### As HCl

13C NMR: (CDCl3+TFA); 100.61 MHz; ppm)

14.70 (q); 23.32 (t); 27.80 (q); 29.39 (t); 29.97 (t); 30.03 (t); 30.10 (t); 30.19 (t); 31.88 (t); 32.54 (t); 34.98 (t); 36.29 (t); 37.24 (t); 51.25 (t); 57.83 (d); 59.88 (s); 127.08 (s); 130.10 (2d); 130.48 (2d); 146.16 (s); 173.09 (s); 176.36 (s).

### EXAMPLE 23

### As HCl

13C NMR: (CDCl3+TFA+CD3OD; 100.61 MHz; ppm)

14.63 (q); 23.29 (t); 27.58 (q); 29.96 (t); 30.00 (t); 30.11 (t); 30.18 (t); 30.34 (t); 31.94 (t); 32.52 (t); 36.31 (t); 36.55 (t); 37.80 (t); 50.08 (t); 59.13 (d); 59.45 (s); 128.14 (s); 129.95 (2d); 130.40 (2d); 145.52 (s); 174.68 (s); 175.34 (s).

### EXAMPLE 25

### As HCl

13C NMR: (CDCl3+TFA; 100.61 MHz, ppm)

26.25 (t); 28.78 (t); 29.21 (t); 33.29 (t); 33.88 (t); 37.26 (t); 50.73 (t); 58.11 (d); 59.62 (s); 68.02 (t); 115.71 (2d); 122.56 (s); 126.55 (d); 129.56 (2d); 129.67 (2d); 131.94 (2d); 137.11 (s); 160.56 (s); 175.04 (s); 176.19 (s).

### EXAMPLE 26

### As HCl

13C NMR: (CDCl3+TFA; 100.61 MHz, ppm)

15.33 (q); 28.30 (t); 29.17 (2t); 33.08 (t); 34.96 (t); 37.21 (t); 51.02 (t); 58.05 (d); 59.69 (s); 65.68 (t); 68.51 (t); 114.45 (d); 115.87 (d); 122.03 (s); 123.23 (s); 128.17 (2d); 130.64 (2d); 131.79 (d); 136.08 (s); 153.11 (s); 160.85 (s); 174.82 (s); 176.00 (s).

### EXAMPLE 27

### 2-methyl-2-[4-(4-nonylbenzylamino)but-2-ynyl]-malonic acid hydrochloride

### Step-I

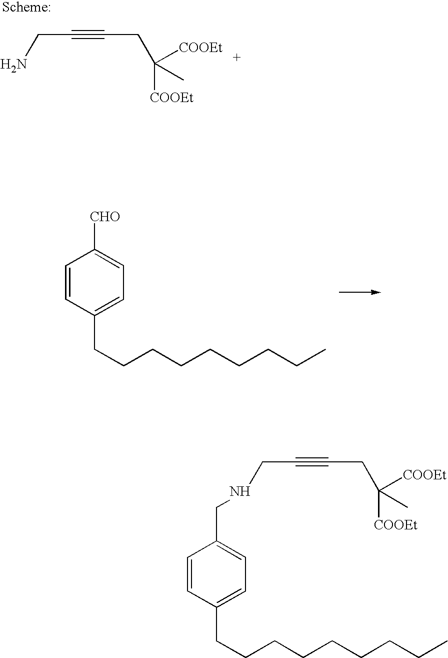

4-Nonylbenzaldehyde (1.44 g, 0.0062 mole) is added to a solution of 2-(4-aminobut-2-ynyl)-2-methylmalonic acid diethyl ester (1.50 g, 0.0062 mole) in dichloromethane at room temperature. Methanol (5 mL) is added to the reaction mixture followed by sodium cyanoborohydride (0.78 g, 0.0124 mole). After one hour stirring at room temperature, sodium cyanoborohydride (0.78 g, 0.0124 mole) is added and the mixture is stirred for 18 h at room temperature. Reaction mixture is concentrated under vacuum, water (15 mL) is added to the residue and extracted with dichloromethane (3×20 mL). The combined organic layers are washed with water (1×15 mL) followed by brine solution (1×15 mL). Removal of dichloromethane gives viscous liquid which is purified by passing through silica gel column (n-hexane:ethyl acetate, 70:30) to get 2-methyl-2-[4-(4-nonylbenzyl-amino)but-2-ynyl]malonic acid diethyl ester.

### Step-II

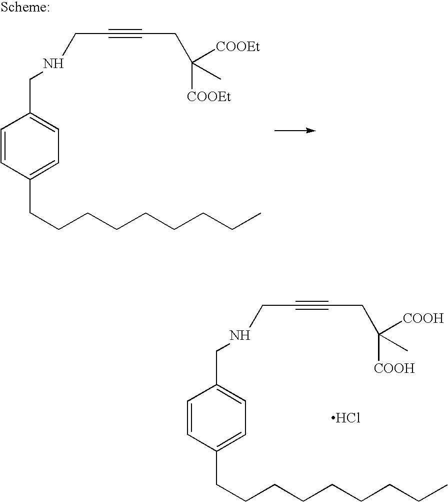

An aqueous solution (5 mL) of sodium hydroxide (0.306 g, 0.0076 mole) is added to an ethanolic solution of 2-methyl-2-[4-(4-nonylbenzylamino)but-2-ynyl]malonic acid diethyl ester (0.70 g, 0.0015 mole). The reaction mixture is then heated at 850 C for 7 h. Ethanol is removed under vacuum, water is added to the residue and acidified to pH ˜1 with 1N HCl. Brine (10 mL) is added to it and extracted with tetrahydrofuran (3×15 mL). The combined organic layers are dried over sodium sulphate and concentrated under vacuum. The crude material is dissolved in minimum volume of acetone and water (5 mL) is added to it. Precipitate thus obtained is filtered and washed with water followed by 10% methanol in diethyl ether and dried under vacuum to get 2-methyl-2-[4-(4-nonylbenzylamino)-but-2-ynyl]malonic acid hydrochloride.

13C NMR: (CDCl3+TFA); 100.61 MHz; δ ppm)

14.70 (q); 20.57 (q); 23.32 (t); 26.62 (t); 29.98 (t); 30.04 (t); 30.12 (t); 30.20 (t); 31.89 (t); 32.54 (t); 36.21 (t); 36.30 (t); 50.40 (t); 53.93 (s); 72.54 (s); 86.31 (s); 126.83 (s); 130.04 (2d); 130.55 (2d); 146.02 (s); 175.11 (2s).

The following further compounds were prepared according to the above procedures:

| Ex. | Compound 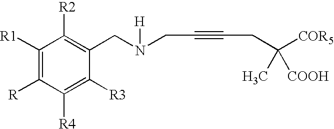 | | | | | | MS (ESI+) |
|---|---|---|---|---|---|---|---|
| 27 | H19C9 | H | H | H | H | OH | 402.3 |
| 28 | H19C90 | Cl | H | H | H | OH | 454.2 & |
| | | | | | | | 452.3 |
| 29 | H17C8 | H | H | H | H | OH | 388.3 |
| 30 | H21C10 | H | H | H | H | OH | 416.3 |
| 31 | H13C6n-4-Ph | H | H | H | F | OH | 454.3 |

### EXAMPLE 28 AS HCL

13C NMR: (CDCl3+TFA); 100.61 MHz; δ ppm)

14.63 (q); 20.40 (q); 23.28 (t); 26.48 (2t); 29.57 (t); 29.87 (t); 29.94 (t); 30.11 (t); 32.49 (t); 36.32 (t); 49.85 (t); 53.88 (s); 70.07 (t); 72.18 (s); 86.47 (s); 114.29 (d); 121.93 (s); 124.30 (s); 130.37 (d); 132.29 (d); 156.75 (s); 175.15 (2s).

### EXAMPLE 29 AS HCL

13C NMR: (CDCl3+TFA); 100.61 MHz; δ ppm)

14.74 (q); 21.24 (q); 23.32 (t); 26.95 (t); 29.93 (t); 30.08 (t); 30.10 (t); 31.93 (t); 32.54 (t); 36.02 (t); 36.33 (t); 49.96 (t); 53.99 (s); 72.64 (s); 86.64 (s); 127.37 (s); 129.87 (2d); 130.74 (2d); 145.48 (s); 175.64 (2s).

### EXAMPLE 30 AS HCL

13C NMR: (TFA+CDCl3; 100.61 MHz; ppm)

14.71 (q); 20.52 (q); 23.33 (t); 26.58 (t); 29.99 (t); 30.05 (t); 30.12 (t); 30.25 (t); 30.28 (t); 31.89 (t); 32.56 (t); 36.23 (t); 36.30 (t); 50.49 (t); 53.93 (s); 72.47 (s); 86.29 (s); 126.69 (s); 130.09 (2d); 130.50 (2d); 146.15 (s); 175.13 (2s).

### EXAMPLE 31 AS HCL

13C NMR: (TFA+CDCl3; 100.61 MHz; ppm)

14.70 (q); 20.52 (q); 23.26 (t); 26.57 (t); 29.74 (t); 31.98 (t); 32.36 (t); 36.33 (t); 36.51 (t); 49.73 (t); 53.94 (s); 72.33 (s); 86.58 (s); 118.34 [d (d), JC—F=23.98 Hz)]; 126.64 (d); 129.27 (2d); 129.35 (2d); 130.33 [s (d), JC—F=7.56 Hz); 131.54 (s (d), JC—F=13.20 Hz)]; 132.30 (d); 132.36 (s); 143.96 (s); 160.35 [s (d), JC—F=249.79 Hz)]; 175.16 (2s).

### Preparation of 4-decyl-2,3-difluorobenzaldehyde

### Step I

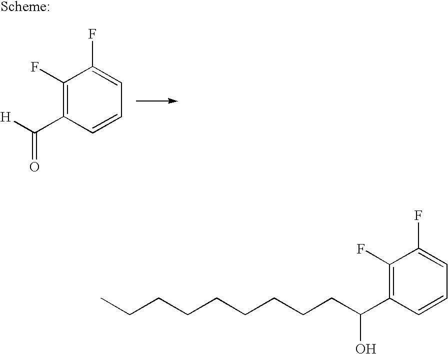

In 250 mL 3-neck R.B flask magnesium turnings (1.014 g, 0.042 mole) in tetrahydrofuran (20 mL) few crystals of iodine is added and reaction mixture is heated to 60-70° C. A solution of 1-bromononane (8.74 g, 0.042 mole) in tetrahydrofuran (10 mL) is added drop wise to the heated reaction mixture and refluxed for 1 hr. Reaction mixture is brought to room temperature and then cooled to −35° C. 2,3-Difluorobenzaldehyde (5.0 g, 0.035 mole) in of tetrahydrofuran (20 mL) is added to reaction mixture drop wise at −35° C. and continued stirring for 1.5 hours. Reaction is quenched with 30 mL 3N HCl at −30° C., aqueous layer is extracted with ethyl acetate (2×30 mL), combined organic extracts are washed with brine and dried over sodium sulphate. Removal of solvent under reduced pressure yielded crude product and is purified by column chromatography (silica gel 230-400 mesh, n-hexane:ethyl acetate 9:1) to furnish 1-(2,3-difluorophenyl)-decan-1-ol.

### Step II

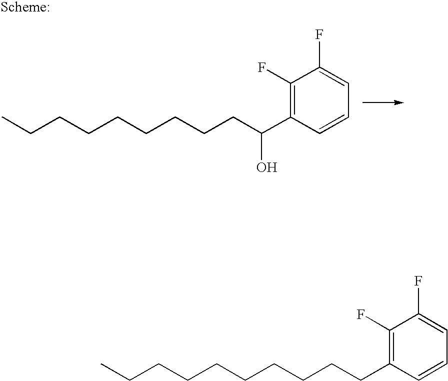

Triethylsilane (2.42 g, 0.21 mole) is added to a solution of 1-(2,3-difluoro-phenyl)decan-1-ol (3.75 g, 0.013 mole) in dichloromethane (30 mL) at room temperature. A solution of titanium tetrachloride (3.16 g, 0.017 mole) in dichloromethane (8 mL) is added to reaction mixture drop wise at 0-5° C. Reaction mixture is brought to room temperature and stirred for 45 mins. After which it is quenched by drop wise addition of D.M. water (15 mL). Aqueous layer is extracted with dichloromethane (2×30 mL), combined organic extracts are washed with brine and dried over sodium sulphate. Removal of solvent under reduced pressure gave crude product which is purified by column chromatography (silica gel 230-400 mesh, n-hexane) to give 1-decyl-2,3-difluoro-benzene.

### Step III

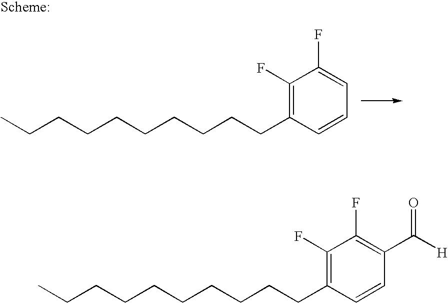

1-Decyl-2,3-difluorobenzene (2.25 g, 0.0089 mole) in tetrahydrofuran (20 mL) is cooled to −70° C. n-Butyl lithium (8.86 mL, 0.014 mole) is added to reaction solution. Reaction mixture is stirred at −70° C. for 1 hr. N,N-dimethyl formamide (1.94 g, 0.027 mole) in tetrahydrofuran (5 mL) is added to the reaction mixture at −7° C., stirred at −70° C. for 30 mins and at −60° C. for 30 mins. Reaction is quenched with D.M. water (15 mL) at −60° C. and allowed to come to room temperature. Aqueous layer is extracted with ethyl acetate (2×20 mL). Combined organic extracts are washed with brine (1×15 mL) and dried over anhydrous sodium sulphate. Removal of solvent under reduced pressure yielded crude product which is purified by column chromatography (silica gel 230-400 mesh, n-hexane) to get 4-decyl-2,3-difluorobenzaldehyde.

4-Decyl-2,3-difluorobenzaldehyde is converted to the corresponding amine by usual procedure viz via mesylate procedure.

### Preparation of 3-cyclopropylamino cyclopentane-1,1-dicarboxylic acid diethyl ester

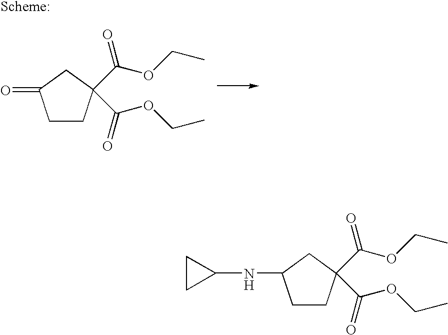

To a stirred solution of cyclopropyl amine (0.749 g, 0.013 mole) in dichloromethane (20 mL), a solution of 3-oxo-cyclopenane-1,1-dicarboxylic acid diethyl ester (2.0 g, 0.0087 mole) in dichloromethane (5 mL) is added at room temperature. The reaction mixture is stirred at room temperature for 2 h and sodium cyanoborohydride (1.65 g, 0.026 mole) is added and allowed to stir at room temperature overnight. The reaction mixture is concentrated under reduced pressure and the residue is treated with D.M. water (20 mL), extracted in ethyl acetate (2×20 mL), combined organic layers are washed with brine (1×10 mL) and dried over sodium sulphate. Removal of solvent furnished 3-cyclopropylamino-cyclopentane-1,1-dicarboxylic acid diethyl ester.

### 3-[Cyclopropyl-(2-fluoro-4′-heptylbiphenyl-4-ylmethyl)amino]cyclopentane-1,1-dicarboxylic acid diethylester

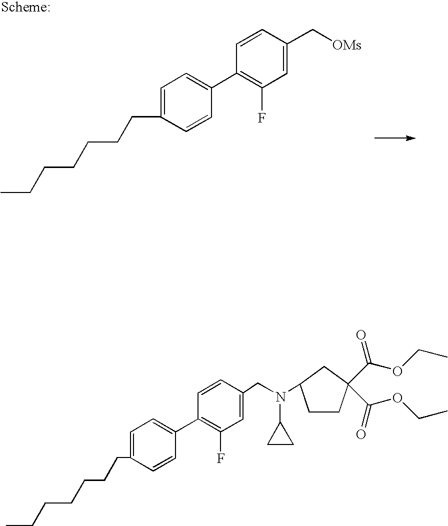

To a stirred solution of 3-cyclopropylaminocyclopentane-1,1-dicarboxylic acid diethyl ester (1.28 g, 0.0047 mole) in N,N-dimethylformamide (10 mL), a solution of methanesulfonic acid 2-fluoro-4′-heptylbiphenyl-4-ylmethyl ester (1.8 g, 0047 mole) in N,N-dimethylformamide (10 mL) is added at room temperature followed by addition of N,N-diisopropyl ethylamine (0.616 g, 0.0047 mole) and heated at 900 C. for 2 hours. The reaction mixture is concentrated under reduced pressure and the residue is quenched with D.M. water (10 mL), aqueous solution is extracted with ethyl acetate (2×20 mL) and dried over anhydrous sodium sufate. Removal of solvent gave viscous liquid which is purified by column chromatography (silica gel 230-400 mesh, n-hexane:ethyl acetate 15:85) to furnish 3-[cyclopropyl-(2-fluoro-4′-heptylbiphenyl-4-ylmethyl)amino]cyclopentane-1,1-dicarboxylic acid diethyl ester. Hydrolysis furnished 47 as white solid.

### Preparation of S(−)-3-amino cyclopentane-1,1-dicarboxylic acid diethyl ester hydrochloride salt

### Step 1

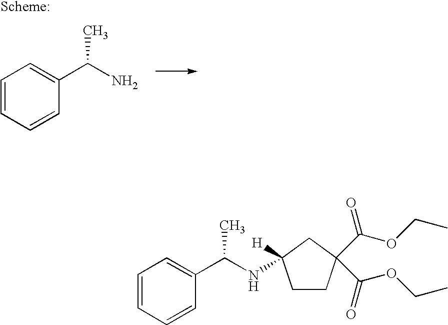

To a stirred solution of 3-oxo-cyclopentane-1,1-dicarboxylic acid diethyl ester (35 g, 0.1535 mole) in ethanol (350 mL) and acetic acid (17.5 mL), S(−)-α-methyl benzylamine (39.6 mL, 0.307 mole) is added at room temperature under nitrogen atmosphere and stirred for 3 hours. Ethanol (650 mL) is added to the reaction mixture and heated at 70° C. Sodium cyanoborohydride (24.1 g) is added in five portions (4.82 g each) at 70° C. After complete addition reaction mixture is heated for 2 hours at 70° C. Reaction mixture is cooled to room temperature and solvent is removed under vacuum. D.M. water (100 mL) is added to the residue and extracted with diethyl ether (2×100 mL), organic layer is dried over anhydrous sodium sulphate and concentrated under vacuum. Column chromatography (silica gel 230-400 mesh, hexane:ethyl acetate, 1:1) yielded diastereomeric mixture which is dissolved in ethyl acetate (290 mL) and cooled to 1° C. Conc. HCl (11.7 mL) in dioxane (24.8 mL) is added, white solid formed is filtered to get the hydrochloride salt of S,S-diastereomer which is recrystallized from hot ethanol. Mother liquor of the hydrochloride salt of the S,S-diastereomer is concentrated and recrystallized to get the hydrochloride salt of the S,R-diastereomer.

### Step II

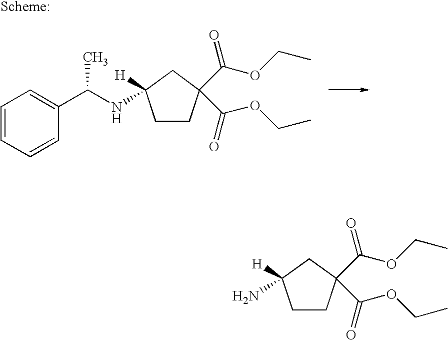

Hydrochloride salt of S,S-diastereomer (6.5 gm) is treated with sat. sodium bicarbonate solution to give the free base of S,S-diastereomer (5.78 g), which is dissolved in ethanol (60 mL), 5% Pd/C (6.7 gm, 50% wet) is added. The mixture is stirred under 40 psi pressure of hydrogen gas at room temperature for 4 hours. Reaction mixture is filtered through celite bed and solvent is removed under vacuum, the material obtained is purified by column chromatography (silica gel 230-400 mesh, dichloromethane:methanol: ammonium hydroxide, 89:10:1) to get S(−)-3-aminocyclopentane-1,1-dicarboxylic acid diethyl ester.

Reductive amination of S(−)-3-aminocyclopentane-1,1-dicarboxylic-acid diethyl ester and R(+)-3-amino cyclopentane-1,1-dicarboxylic-acid diethyl ester with the aldehyde, followed by hydrolysis yielded enantiomer B (e.g. 73) and enantiomer A (e.g 72) respectively.

### Preparation of 2-(4-octylphenyl)ethylamine

### Step I

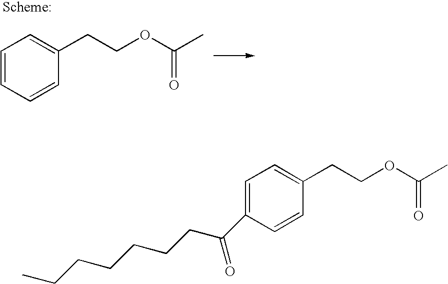

To a heterogeneous solution of aluminium chloride (22.070 g, 0.165 mole) in dichloroethane (180 mL), is added at 00 C a mixture of phenyl ethylacetate (18.37 g, 0.112 mole) and octanoyl chloride (18.2 g, 0.112 mole) in dichloroehtane (80 mL). The reaction mixture is brought to room temperature and stirred for 2 hours. Reaction is quenched with 6N HCl (100 mL) at 10° C., aqueous layer is extracted with ethyl acetate (2×100 mL), combined organic layer is washed with brine (1×50 mL) and dried over anhydrous sodium sulphate. Removal of solvent furnished viscous liquid which is purified by column chromatography (silica gel 230-400 mesh, n-hexane:ethyl acetate 90:10) to get acetic acid 2-(4-octano-ylphenyl)ethyl ester.

### Step II

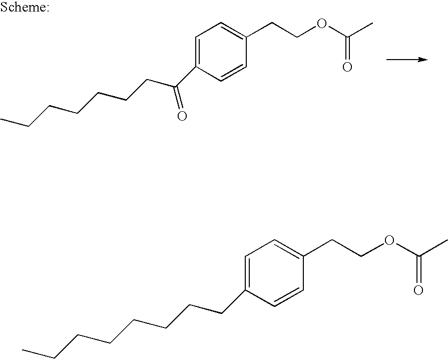

Triethylsilane (13 mL) is added drop wise to a solution of acetic acid 2-(4-octanoyl phenyl)ethyl ester (6.5 g, 0.022 mole) and trifluoroacetic acid (20 mL) at room temperature and stirred overnight. Reaction mixture is concentrated under reduced pressure to get the crude mass, which is purified by column chromatography (silica gel 230-400 mesh, n-hexane:ethyl acetate, 95:05) to get acetic acid 2-octylphenyl)ethyl ester

### Step III

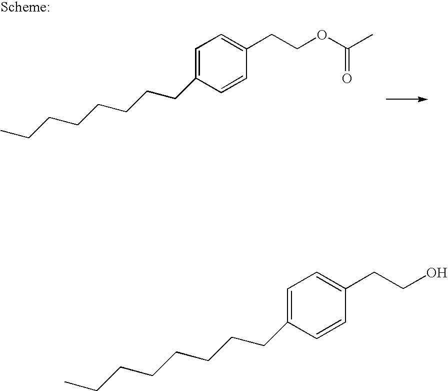

A solution of acetic acid 2-(4-octylphenyl)ethyl ester (4.0 g, 0.014 mole) in methanol (20 mL) is treated with sodium methoxide solution at room temperature. Reaction mixture is heated to reflux for 2 hours. Methanol is evaporated under reduced pressure, D.M. water (20 mL) is added to the residue and extracted with ethyl acetate (2×20 mL), combined organic layers is washed with brine (1×20 mL) and dried over anhydrous sodium sulphate. Removal of solvent furnished crude product 2-(4-octyl-phenyl)ethanol, which is used as such for next step.

### Step IV

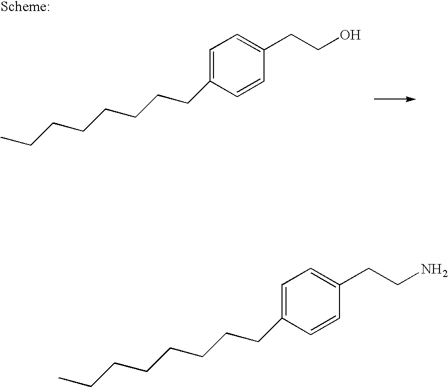

Triethylamine (1.85 g, 0.018 mL) is added to a solution of 2-(4-octylphenyl)ethanol (3.3 g, 0.014 mole) in dichloromethane (33 mL) at room temperature. A solution of methanesulphonyl chloride (1.77 g, 0.015 mole) is added to above solution at 10° C. Reaction mixture is brought to room temperature and stirred for 1 h. The reaction is quenched with water (15 mL) at 100 C and extracted with MDC (2×10 mL). Combined organic layer is washed with saturated sodium bicarbonate solution (1×20 mL) followed by brine (1×20 mL) and dried over anhydrous sodium sulphate. Removal of solvent furnished crude methanesulfonic acid 2-(4-octylphenyl)ethyl ester. This crude material is dissolved in methanol (10 mL), methanolic ammonia (100 mL) is added to it and stirred at room temperature for overnight. Removal of methanol under reduced pressure furnished crude product which is purified by column chromatography (silica gel 230-400 mesh, dichloromethane: methanol, 80:20) to get 2-(4-octylphenyl)ethylamine. Reductive amination with 3-oxocyclopentane-1,1-dicarboxylic acid diethyl ester followed by hydrolysis yielded 101

### Preparation of 3-[(4-Decyl-2,3-difluorobenzyl)ethoxycarbonylamino]cyclopentane 1,1-dicarboxylic acid

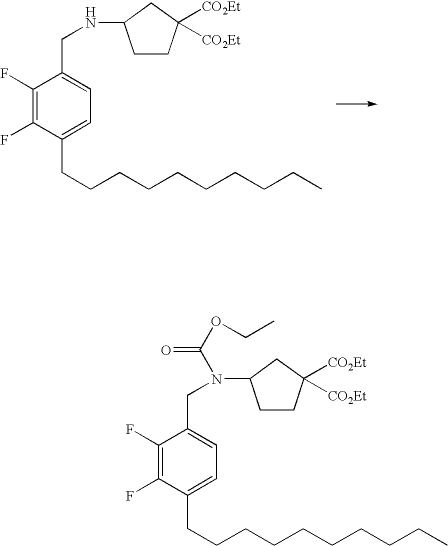

### Step-I

N,N-Di-isopropylethylamine (0.516 mL, 0.0029 mole) is added to a stirred solution of 3-(4-decyl-2,3-difluorobenzylamino)cyclopentane-1,1-dicarboxylic diethyl ester (0.980 g, 0.0019 mole) in tetrahydrofuran (10 mL) at room temperature. Ethyl chloroformate (0.278 mL, 0.0029 mole) is introduced into the reaction mixture and stirred for 1 hr at room temperature. Reaction mixture is concentrated under vacuum, D.M. water (10 mL) is added and aqueous layer is extracted in ethyl acetate (3×15 mL). Combined organic layer is washed with brine solution (1×15 mL) and dried over anhydrous sodium sulphate. Removal of solvent furnished colourless liquid, purified by column chromatography (silica gel, 230-400 mesh, toluene:ethyl acetate, 93:7) to get 3-[(4-decyl-2,3-difluorobenzyl)ethoxycarbonyl-amino]cyclopentane-1,1-dicarboxylic diethyl ester.

### Step-II

An aqueous solution of (3 mL) of sodium hydroxide (0130 g, 0.0032 mole) is added to a stirred ethanolic solution of 3-[(4-decyl-2,3-difluorobenzyl)ethoxycarbonyl-amino]cyclopentane-1,1-dicarboxylic diethyl ester (0.370 g, 0.00065 mole). Reaction mixture is heated at 800° C. for 4.5 hours. Ethanol is removed under vacuum, D.M. water (10 mL) is added to the residue and the solution is acidified to pH ˜1 with 1N HCl. Brine solution (5 mL) is added, aqueous layer is extracted with tetrahydrofuran (3×10 mL). Removal of solvent under reduced pressure furnished yellow solid which is washed with methylenedichloride to get 3-[(4-decyl-2,3-difluorobenzyl)ethoxycarbonylamino]cyclopentane-1,1-dicarboxylic acid.

### Preparation of 3[(4-decyl-2,3-difluorobenzyl)ethoxycarbonylamino]cyclopentane-1,1-dicarboxylic acid ethyl ester

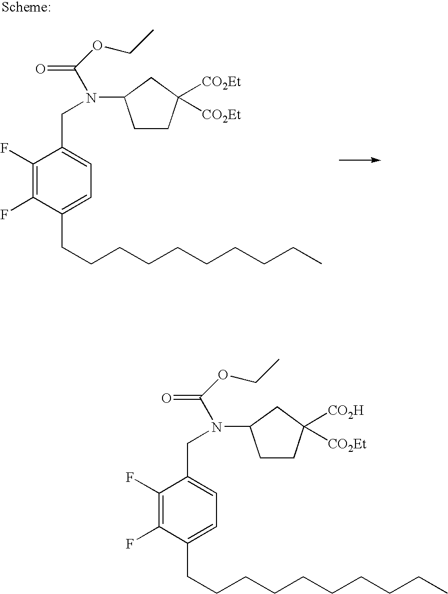

An aqueous solution of (3 mL) of sodium hydroxide (0.050 g, 0.0012 mole) is added to a stirred ethanolic solution of 3-[(4-decyl-2,3-difluorobenzyl)ethoxycarbonyl-amino]cyclopentane-1,1-dicarboxylic diethyl ester (0.50 g, 0.0009 mole). Reaction mixture is heated at 450° C. for 30 mins. Ethanol is removed under vacuum, D.M. water (10 mL) is added to the residue and the solution is acidified to pH ˜6 with 1N HCl. Brine solution (5 mL) is added and aqueous layer is extracted with tetrahydrofuran (3×15 mL). Removal of solvent under reduced pressure furnished light yellow liquid which is purified by column chromatography (silica gel, 230-400 mesh, toluene:ethylacetate, 50:50) to get two isomers of 3-[(4-decyl-2,3-difluorobenzyl)ethoxycarbonylamino]cyclopentane 1,1-dicarboxylic ethyl ester.

### Preparation of 2-Methyl-2-[4-(4-nonylphenyl)but-2-enyl]malonic acid

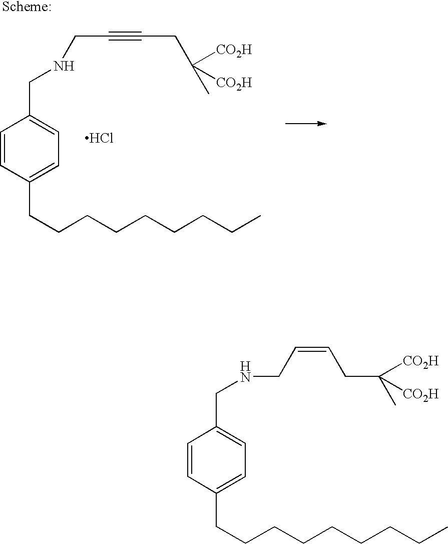

A mixture of pyridine (0.068 mL, 0.0008 mole) and 2-methyl-2-[4-(4-nonylbenzylamino)but-2-ynyl]-malonic acid hydrochloride (0.37 g, 0.0008 mole) in tetrahydrofuran (15 mL) are taken in a hydrogenation apparatus. Lindlar's catalyst (50 mg) is added to reaction mixture and stirring is continued for 2.5 hours under hydrogen pressure at 2 kg/cm². Reaction mixture is filtered off using celite bed and washed with tetrahydrofuran (2×10 mL). Combined filterate is concentrated under reduced pressure and the resulting solid is purified by column chromatography (silica gel 230-400 mesh, ethyl acetate:methanol, 85:15) to get 2-methyl-2-[4-(4-nonylphenyl)but-2-enyl]malonic acid (70).

Following compounds are prepared by reductive amination, N-alkylation (where ever required) followed by alkaline hydrolysis.

| | Compound | | | | | | | |
|---|---|---|---|---|---|---|---|---|
| | 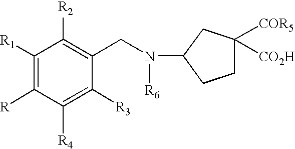 | | | | | | | |
| Ex. | R | R1 | R2 | R3 | R4 | R5 | R6 | MS (ESI+) |

| | | | | | | | | |
|---|---|---|---|---|---|---|---|---|
| 32 | H15C7n-4-Ph | H | H | H | F | OH | H | 456.3 |
| 33 | C9H19 | H | H | H | H | NH2 | H | 389.3 |
| 34 | C10H21O | Cl | H | H | H | OH | H | 452.6, |
| | | | | | | | | 454.5 |
| 35 | C8H17O | Cl | H | H | H | OH | H | 424.4, |
| | | | | | | | | 426.5 |
| 36 | C9H19 | H | H | H | H | NH2 | H | 389.3 |
| 37 | C9H19 | H | H | H | H | OH | 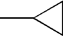 | 430.3 |
| 38 | H15C7n-4-Ph | H | H | H | F | OH | CH3 | 470.3 |
| 39 | C9H19 | F | F | H | H | OH | H | 426.2 |
| 40 | C9H19 | F | F | H | H | OEt | H | 454.3 |
| 41 | C9H19 | H | H | H | H | 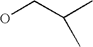 | H | 446.4 |
| 42 | C9H19 | H | H | H | H | OEt | CH3 | 432.5 |
| 43 | C9H19 | H | H | H | H | OMe | H | 404.3 |
| 44 | C9H19 | H | H | H | H | OCH2Ph | H | 480.4 |
| 45 | H17C8n-4-Ph | H | H | H | F | OEt | H | 498.3 |
| 46 | H17C8n-4-Ph | H | H | H | F | OH | H | 470.3 |
| 47 | H15C7n-4-Ph | H | H | H | F | OH | 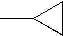 | 496.3 |
| 48 | C9H19 | H | H | H | H | 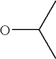 | H | 432.3 |
| 49 | C9H19 | F | F | H | H | NHCH3 | H | 439.3 |
| 50 | C9H19 | F | F | H | H | NHCH3 | H | 439.3 |
| 51 | C9H19 | H | H | H | H | 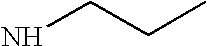 | H | 431.3 |
| 52 | C9H19 | H | H | H | H | 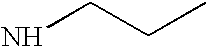 | H | 431.3 |
| 53 | C10H21 | F | F | H | H | OH | H | 440.1 |
| 54 | C10H21 | H | H | H | H | NHCH3 | H | 417.4 |
| 55 | C10H21 | H | H | H | H | NHCH3 | H | 417.3 |
| 56 | C9H19 | H | H | H | H | 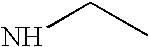 | H | 417.3 |
| 57 | C9H19 | H | H | H | H | 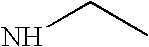 | H | 417.3 |
| 58 | C9H19 | H | H | H | H | 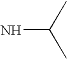 | H | 431.4 |
| 59 | C9H19 | H | H | H | H | 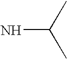 | H | 431.4 |
| 60 | C10H21 | F | F | H | H | NHCH3 | H | 453.2 |
| 61 | C9H19 | F | F | H | H | NHCH3 | CH3 | 453.4 |
| 62 | C9H19 | F | F | H | H | NHCH3 | CH3 | 453.4 |
| 63 | C10H21 | H | H | H | H | OH | CH3 | 418.4 |
| 64 | C9H19 | H | H | H | H | 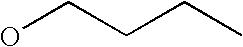 | H | 446.4 |
| 65 | C9H19 | H | H | H | H | 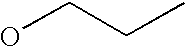 | H | 432.5 |
| 66 | C9H19 | H | H | H | H | 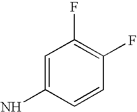 | H | 501.3 |
| 67 | C9H19 | F | F | H | H | OH | CH3 | 440.4 |
| 68 | C10H21 | F | F | H | H | 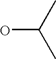 | H | 482.4 |
| 69 | C10H21 | H | H | H | H | 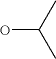 | H | 446.5 |
| 70 | H15C7n-4-Ph | H | H | H | F | NHCH3 | H | 469.3 |
| 71 | H15C7n-4-Ph | H | H | H | F | NHCH3 | H | 469.3 |
| 72 | C10H21 | H | H | H | H | OH | H | 404.3 |
| 73 | C10H21 | H | H | H | H | OH | H | 404.3 |
| 74 | C10H21O | Cl | H | H | H | OH | CH3 | 468.2, |
| | | | | | | | | 470.3 |
| 75 | C9H19O | Cl | H | H | H | OH | CH3 | 454.2, |
| | | | | | | | | 456.4 |
| 76 | C9H19 | H | H | H | H | 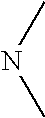 | H | 417.3 |
| 77 | C8H17 | H | H | H | H | NHEt | H | 403.4 |
| 78 | C8H17 | H | H | H | H | NHEt | H | 403.4 |
| 79 | C8H17 | H | H | H | H | NHCH3 | H | 389.3 |
| 80 | C8H17 | H | H | H | H | NHCH3 | H | 389.3 |
| 81 | C8H17 | H | H | H | H | 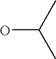 | H | 418.6 |
| 82 | C10H21 | F | F | H | H | OH | CH3 | 454.5 |
| 83 | C10H21, | F | F | H | H | OH | H | 438.1 |
| 84 | C10H21, | F | F | H | H | OH | H | 438.1 |
| 85 | H15C7n-4-Ph | H | H | H | F | OH | H | 456.1 |
| 86 | C10H21 | H | H | H | H | OEt | H | 432.3 |
| 87 | C10H21 | H | H | H | H | OEt | H | 432.3 |
| 88 | C10H21 | H | H | H | H | OEt | CH3 | 446.4 |
| 89 | C10H21 | F | F | H | H | OH | CH3 | 454.3 |
| 90 | C10H21 | F | F | H | H | OEt | CH3 | 482.3 |
| 91 | C10H21 | F | F | H | H | OH | CH3 | 453.8 |
| 92 | C10H21 | F | F | H | H | OEt | CH3 | 481.8 |
| 93 | C10H21 | H | F | F | H | OH | H | 440.1 |
| 94 | C10H21 | H | F | F | H | OH | CH3 | 454.1 |
| 95 | C10H21 | F | F | H | H | OH | COOEt | 512.0 |
| 96 | C10H21 | F | F | H | H | OEt | COOEt | 540.1 |
| 97 | C10H21 | F | F | H | H | OEt | COOEt | 540.1 |
| 98 | C10H21 | F | F | H | H | OH | COOMe | 498.1 |
| 99 | C10H21 | F | F | H | H | OEt | COOMe | 526.2 |

| | | |
|---|---|---|
| 100 | 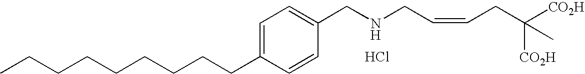 | 404.3 |
| 101 | 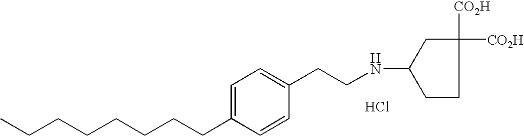 | 390.4 |
| 102 | 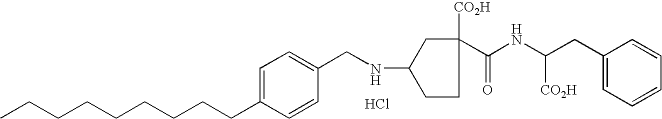 | 537.4 |
| 103 | 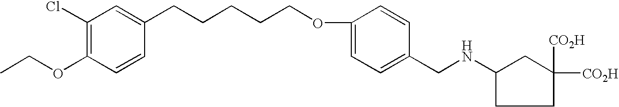 | 503.3, 502.4 |
| 104 | 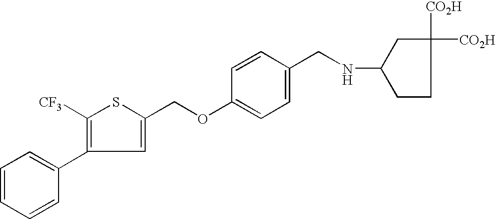 | 518 |
| 105 | 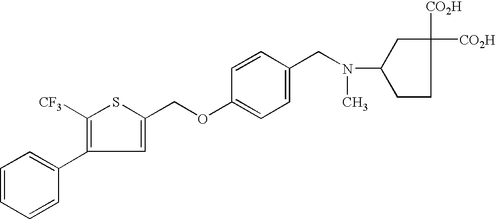 | 532 |

### Preparation of 3-(4-Decylbenzylamino)cyclobutane-1,1-dicarboxylic acid diethyl ester

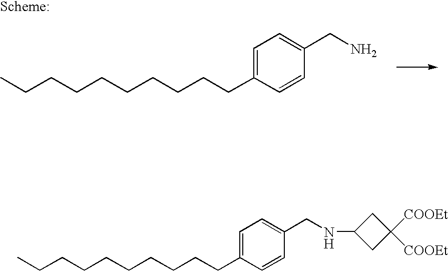

To a stirred solution of 4-decylbenzylamine (0.80 g, 0.003 mole) in dichloro-methane (20 mL) and methanol (2 mL), a solution of 3-oxocyclobutane-1,1-dicarboxylic acid diethyl ester (0.7 g, 0.0032 mole) (Chem. Ber., 1957, 90, 1424-1432; J. Med. Chem., 1990, 33, 2905-2915.) in dichloromethane (5 mL) is added at Concentrated under reduced pressure and residue is treated with D.M. water (10 mL). Aqueous layer is extracted in ethyl acetate (2×20 mL). Combined organic layer is washed with brine (1×10 mL) and dried over anhydrous sodium sulphate. Removal of solvent gives viscous liquid which is purified by column chromatography (silica gel 230-400 mesh, n-hexane:ethyl acetate 70:30) to get 3-(4-decylbenzylamino)cyclobutane-1,1-dicarboxylic acid diethyl ester.

### Preparation of 3-[(4-decylbenzyl)methylamino]cyclobutane-1,1-dicarboxylic acid diethyl ester

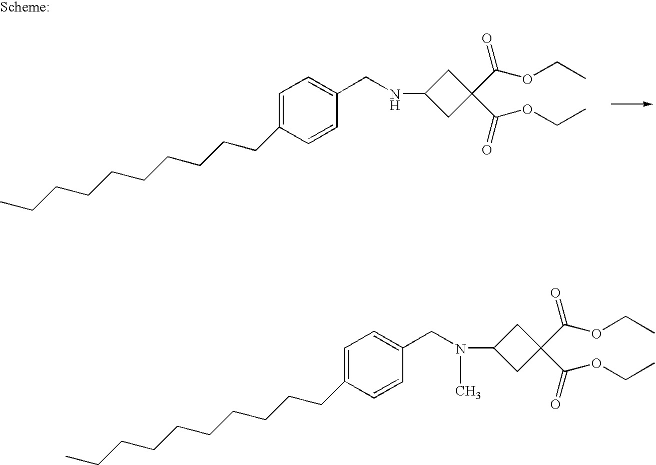

To a stirred solution of 3-(4-decylbenzylamino)cyclobutane-1,1-dicarboxylic acid diethyl ester (1.4 g, 0.00314 mole) in acetone (20 mL) is added potassium carbonate (0.65 g, 0.0047 mole). The reaction mixture is cooled to 5-100 C and methyl iodide (0.22 mL, 0.00345 mole) is added, reaction mixture is brought to room temperature and stirred for 4 hours. Concentrated under reduced pressure, treated with D.M. water (5 mL) and extracted with ethyl acetate (2×20 mL). Combined organic layer is washed with brine (1×10 mL) and dried over anhydrous sodium sulphate. Removal of solvent furnished viscous liquid which is purified by column chromatography (silica gel 230-400 mesh, ethyl acetate:hexane 15:85) to get 3-[(4-decylbenzyl)methyl-amino]cyclobutane-1,1-dicarboxylic acid.

### Preparation of 3-hydroxycyclobutane-1,1-dicarboxylic acid ethyl ester

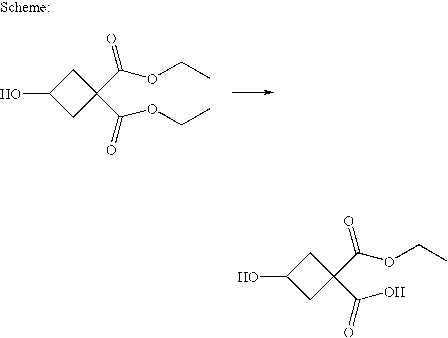

To a stirred solution of 3-hydroxycyclobutane-1,1-dicarboxylic acid diethyl ester (4.2 g, 0.019 mole) in ethanol (30 mL), a solution of sodium hydroxide (0.85 g, 0.021 mole) in water (12 mL) is added at room temperature. The reaction mixture is heated to 65-700 C for 40 mins. The mixture is concentrated under reduced pressure and the residue is quenched with D.M water (10 mL). The aqueous layer, after washing with ethyl acetate (1×15 mL) is acidified to pH ˜2 by drop wise addition of dil. HCl. Aqueous layer is then extracted with ethyl acetate (3×15 mL). Combined organic extract is washed with brine and dried over anhydrous sodium sulphate. Removal of solvent furnished 3-hydroxycyclobutane-1,1-dicarboxylic acid ethyl ester.

### Preparation of 3-oxocyclobutane-1,1-dicarboxylic acid ethyl ester

3-hydroxycyclobutane-1,1-dicarboxylic acid ethyl ester is oxidised according to the literature procedure (J. Med. Chem. 1990, 33, 2905-2915) to get the corresponding 3-oxocyclobutane-1,1-dicarboxylic acid ethyl ester.

### Preparation of 1-methylcarbamoyl-3-oxocyclobutane carboxylic acid ethyl ester

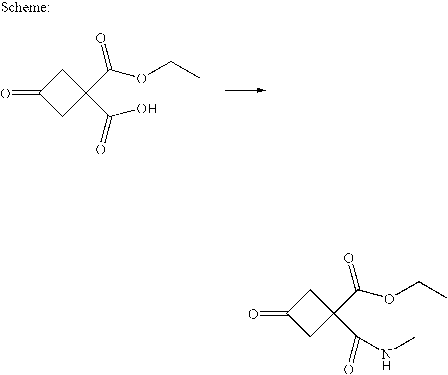

Isobutyl chloroformate (1.93 mL, 0.00148 mole) is added to a stirred solution of 3-oxocyclobutane-1,1-dicarboxylic acid ethyl ester (2.3 g, 0.012 mole) and N-methyl-morpholine (2.04 mL, 0.0185 mole) in tetrahydrofuran (23 mL) at −10° to −15° C. temperature. After 1.5 hours stirring, methyl amine (0.96 mL) is added and stirring is continued for 1.5 hours at −10° to −15° C. It is then quenched with D.M. water (5 mL) and extracted with ethyl acetate (2×15 mL). Combined organic extract is washed with saturated sodium bicarbonate solution and dried over anhydrous sodium sulphate. Removal of solvent under reduced pressure yields viscous liquid which is purified by column chromatography (silica gel 230-400 mesh, hexane:ethyl acetate 3.7) to get 3-oxo-1-methylcarbamoyl cyclobutanecarboxylic acid ethyl ester.

Hydrolysis of ester following the procedure as mentioned for the corresponding cyclopentane derivative furnish corresponding acid derivative.

Similar way following compounds are also prepared:

| | Compound | | | | | | | |
|---|---|---|---|---|---|---|---|---|
| | 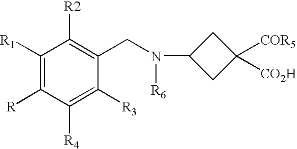 | | | | | | | |
| Ex. | R | R1 | R2 | R3 | R4 | R5 | R6 | MS (ESI+) |
| 106 | H15C7n-4-Ph | H | H | H | F | OH | H | 442.5 |
| 107 | C9H19 | H | H | H | H | OH | H | 376.3 |
| 108 | C10H21 | H | H | H | H | OH | H | 390.3 |
| 109 | C10H21 | H | H | H | H | OH | CH3 | 404.2 |
| 110 | C10H21 | F | F | H | H | OH | H | 426.4 |
| 111 | H15C7n-4-Ph | H | H | H | F | NHCH3 | H | 455.6 |
| 112 | H15C7n-4-Ph | H | H | H | F | NHCH3 | H | 455.5 |
| 113 | C10H21 | H | H | H | H | OEt | CH3 | 432.3 |
| 114 | C10H21 | F | F | H | H | OH | CH3 | 440.3 |
| 115 | C10H21 | F | F | H | H | OEt | CH3 | 468.3 |

| | | |
|---|---|---|
| 116 | 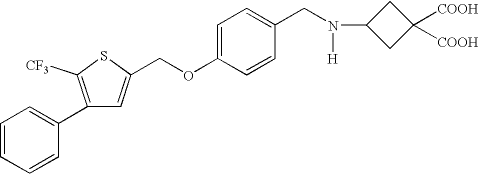 | 506.1 |

### Preparation of 1-(2,3-difluoro-4-nonylbenzyl)piperidine-4,4-dicarboxylic acid ethyl ester

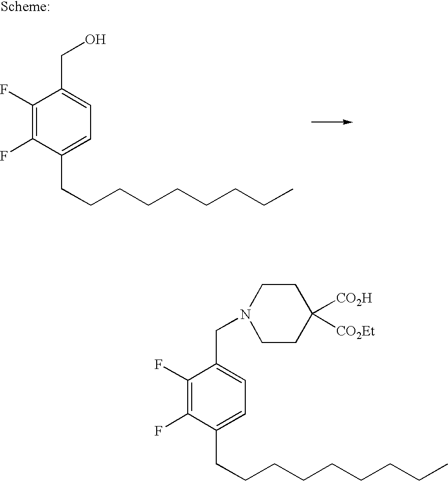

### Step-1

Triethyl amine (1.3 mL, 0.0094 mole) is added to a stirred dichloromethane solution (12 mL) of 2,3-difluoro-4-nonylbenzyl alcohol (1.27 g, 0.0047 mole) at room temperature. Reaction mixture is cooled to 0°-5° C. followed by slow addition of methanesulphonyl chloride (0.55 mL, 0.0070 mole). The mixture is then stirred at room temperature for total 5.5 hours. D.M. water (10 mL) is added and organic layer is separated. Aqueous layer is extracted with dichloromethane (3×10 mL).

Combined organic layer is washed with water (1×15 mL) followed by brine solution (1×15 mL) and dried over anhydrous sodium sulphate. Removal of solvent furnished 1.32 g of light yellow liquid.

### Step-II

N,N-dimethyl formamide solution (5 mL) of piperidine-4,4-dicarboxylic acid diethyl ester (1.0 g, 0.0045 mole) (Bio-org & Med. Chem. Lett., 1997, 7, 1311-1316; Synth. Commun. 1981, 11, 17-23), is added to a stirred N,N-dimethyl formamide solution (5 mL) of mesylated derivative (1.32 g) at room temperature. N,N-diisopropyl ethyl amine (1.0 mL, 0.0057 mole) is added to the mixture and heated at 900 C for 1 hr. Reaction mixture is concentrated under reduced pressure, D.M. water (10 mL) is added to the residue and aqueous layer is extracted with ethyl acetate (3×15 mL). Combined organic layer is washed with D.M. water (1×10 mL) followed by brine solution (1×10 mL) and dried over anhydrous sodium sulphate. Removal of solvent gives 2.3 g of light brown viscous liquid, which is purified by column chromatography (silica gel 230-400 mesh, n-hexane:ethyl acetate, 85:15) to get 1-(2,3-difluoro-4-nonyl-benzyl)piperidine-4,4-dicarboxylic acid diethyl ester.

### Step-III

An aqueous solution (3 mL) of sodium hydroxide (0.13 g, 0.0033 mole) is added to a stirred ethanolic solution (7 mL) of 1-(2,3-difluoro-4-nonylbenzyl)piperidine-4,4-dicarboxylic acid diethyl ester (1.32 g, 0.0027 mole). The mixture is heated at 600 C for two hours. Ethanol is removed, D.M. water (15 mL) is added to the residue and the solution is acidified to pH ˜6 with 1N HCl. Solid thus formed during acidification is filtered-off, washed with water (2×5 mL) and dried under vacuum. This crude solid is purified by column chromatography (silica gel 230-400 mesh, dichloromethane: methanol, 80:20) to get 1-(2,3-difluoro-4-nonylbenzyl)piperidine-4,4-dicarboxylic acid ethyl ester.

### Preparation of 1-(2,3-difluoro-4-nonylbenzyl)-4-ethylcarbamoylpiperidine-4-carboxylic acid

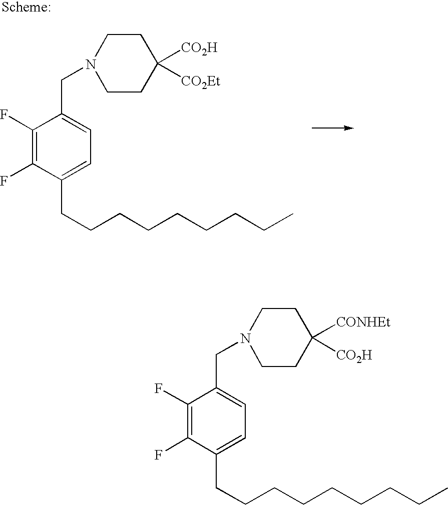

### Step-I

N-Methyl morpholine (0.34 mL, 0.003 mole) is added to a stirred solution of 1-(2,3-difluoro-4-nonyl benzyl)piperidine-4,4-dicarboxylic acid ethyl ester (0.77 g, 0.0017 mole) in tetrahydrofuran (10 mL) at room temperature. Reaction mixture is cooled to −200 C and isobutyl chloroformate (0.31 mL, 0.0023 mole) is added slowly to this mixture. It is allowed to attain room temperature and stirred for two more hours. Ethyl amine (70% aqueous solution, 0.44 mL, 0.0067 mole) is added to the mixture at 00-50 C and stirred at this temperature for 10 minutes and then allowed to stirr at room temperature for one hour. D.M. water (10 mL) is added and stirred for five minutes. Organic layer is separated and aqueous layer is extracted with ethyl acetate (3×15 mL). Combined organic layer is washed with water (1×10 mL) followed by brine solution (1×10 mL) and dried over anhydrous sodium sulphate. Removal of solvent gives viscous liquid, which is purified by column chromatography (silica gel 230-400 mesh, ethyl acetate:n-hexane, 70:30) to get 1-(2,3-difluoro-4-nonylbenzyl)-4-ethylcarbamoylpiperidine-4-carboxylic acid ethyl ester.

### Step-II

An aqueous solution (2 mL) of lithium hydroxide (0.060 g, 0.0014 mole) is added to a stirred ethanolic solution (5 mL) of 1-(2,3-difluoro-4-nonylbenzyl)-4-ethyl-carbamoylpiperidine-4-carboxylic acid ethyl ester (0.48 g, 0.001 mole). The mixture is heated at 500 C for 3 hours. Ethanol is removed, D.M. water (5 mL) is added to the residue and the solution is acidified to pH ˜6 with 1N HCl. Solid thus formed during acidification is filtered-off, washed with water (2×5 mL) and dried under vacuum. This crude solid is purified by column chromatography (silica gel 230-400 mesh, dichloromethane:methanol, 80:20) to get 1-(2,3-difluoro-4-nonylbenzyl)-4-ethylcarbamoylpiperidine-4-carboxylic acid.

### Preparation of 1-(4-decylbenzyl)piperidine-4,4-dicarboxylic acid

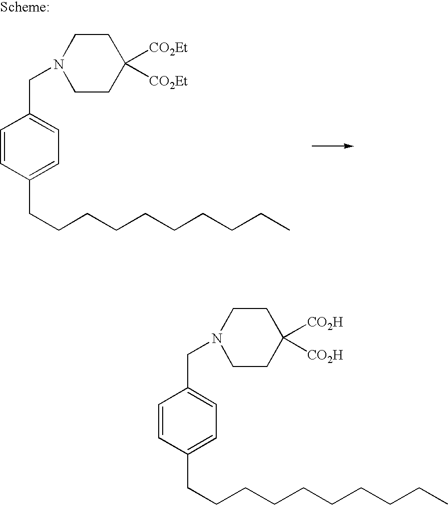

An aqueous sodium hydroxide (0.30 g, 0.0075 mole) solution (5 mL) is added to a stirred ethanolic solution (8 mL) of 1-(4-decyl-benzyl)piperidine-4,4-dicarboxylic acid diethyl ester (0.70 g, 0.0015 mole), reaction mixture is heated at reflux for four hours. Ethanol is removed under vacuum, D.M. water (15 mL) is added to the residue and the solution is acidified to pH ˜2 with 3N HCl. Compound thus precipitated during acidification is filtered and washed with a mixture of methanol-diethyl ether (20:80) to get 1-(4-decylbenzyl)piperidine-4,4-dicarboxylic acid.

Similar way following compounds are also prepared:

| | Compound | | | | | | |
|---|---|---|---|---|---|---|---|
| | 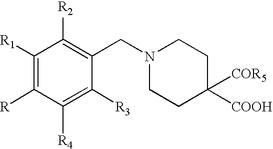 | | | | | | |
| Ex. | R | R1 | R2 | R3 | R4 | R5 | MS (ESI+) |
| 117 | C10H21 | H | H | H | H | OH | 404.4 |
| 118 | C9H19 | H | H | H | H | OH | 390.2 |
| 119 | C10H21 | F | F | H | H | OH | 440.2 |
| 120 | C8H17 | H | H | H | H | OH | 376.1 |
| 121 | OC9H19 | Cl | H | H | H | OH | 440.1, 442.2 |
| 122 | C7H15n-4-Ph | H | H | H | F | OH | 454.2 |
| 123 | C10H21 | H | H | H | H | OEt | 432.8 |
| 124 | OC10H21 | Cl | H | H | H | OEt | 482.1, 484.1 |
| 125 | C10H21 | F | F | H | H | OEt | 468.5 |
| 126 | C7H15n-4-Ph | H | H | H | F | OEt | 484.1 |
| 127 | C10H21 | F | F | H | H | OMe | 454.5 |
| 128 | C10H21 | F | F | H | H | 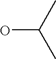 | 482.5 |
| 129 | C10H21 | H | H | H | H | NHEt | 431.5 |
| 130 | C10H21 | H | H | H | H | 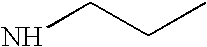 | 445.5 |
| 131 | C9H19 | F | F | H | H | NHEt | 453.5 |

| | | |
|---|---|---|
| 132 | 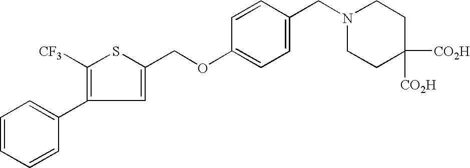 | 518 |

### Preparation of 4-(4-decyl-2,3-difluorobenzylamino)cyclohexane-1,1-dicarboxylic acid diethyl ester

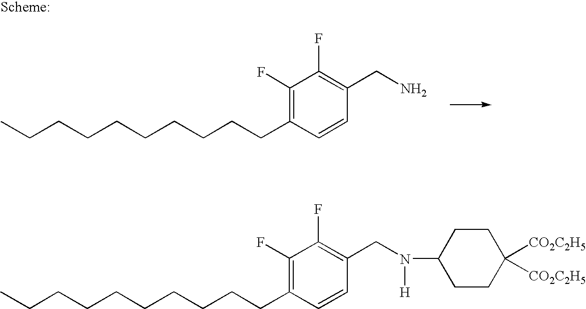

To a stirred solution of 4-decyl-2,3-difluorobenzylamine (2.47 g, 0.0087 mole) in dichloromethane (50 mL) and methanol (50 mL), a solution of 4-oxocyclohexane-1,1-dicarboxylic acid diethyl ester (2.11 g, 0.0087 mole) (Helv. Chim. Acta., 1944, 27, 793-800; Tetrahedron, 1958, 3, 175-177). in dichloromethane (5 mL) is added at room temperature. The reaction mixture is stirred at room temperature for 2 hours and sodium cyanoborohydride (0.68 g, 0.0218 mole) is added in two portions within 1 hr interval. The reaction mixture is allowed to stir at room temperature overnight. The mixture is concentrated under reduced pressure and the residue is treated with D.M water (20 mL) and aqueous layer is extracted in ethyl acetate (2×20 mL). Combined organic layer is washed with brine (1×10 mL) and dried over anhydrous sodium sulphate. Removal of solvent furnished viscous liquid which is purified by column chromatography (silica gel 230-400 mesh, n-hexane:ethyl acetate 60:40) to get 4-(4-decyl-2,3-difluorobenzylamino)cyclohexane-1,1-dicarboxylic acid diethyl ester.

### Preparation of 4-(4-decyl-2,3-difluorobenzylamino)cyclohexane-1,1-dicarboxylic acid

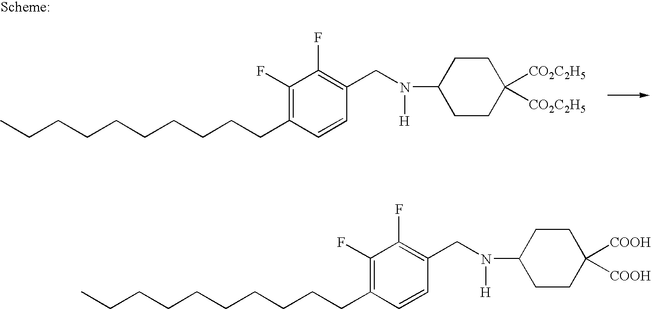

To a stirred solution of 4-(4-decyl-2,3-difluorobenzylamino)cyclohexane-1,1-dicarboxylic acid diethyl ester (0.45 g, 0.00088 mole) in ethanol (5 mL), a solution of sodium hydroxide (0.21 g, 0.0053 mole) in water (6 mL) is added at room temperature. The reaction mixture is heated under reflux for 8 hours, concentrated under reduced pressure and the residue is treated with water (2 mL). The aqueous solution is acidified to pH ˜2 by drop wise addition of dil. HCl. Solid obtained is filtered, washed with methanol:ether (10:90) (10 mL) to get 4-(4-decyl-2,3-difluorobenzylamino)cyclohexane-1,1-dicarboxylic acid

### Preparation of 4-[(4-decyl-2,3-difluorobenzyl)methylamino]cyclohexane-1,1-dicarboxylic acid diethyl ester

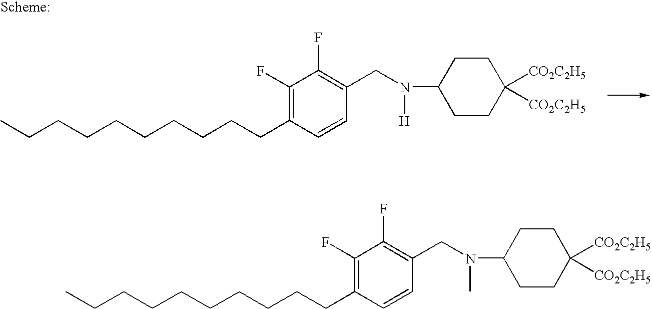

To a stirred solution of 4-(4-decyl-2,3-difluorobenzylamino)cyclohexane-1,1-dicarboxylic acid diethyl ester (2.1 g, 0.0041 mole) in acetone (42 mL), potassium carbonate (0.85 g, 0.0061 mole) is added at room temperature. The reaction mixture is cooled to 10° C. and methyl iodide (0.64 g, 0.0045 mole) is added. Reaction mixture is allowed to stir overnight at room temperature, concentrated under reduced pressure and the residue is treated with water (20 mL). The aqueous layer is extracted in ethyl acetate (2×20 mL). Combined organic layer are washed with brine (1×10 mL) and dried over sodium sulphate. Removal of solvent furnished a viscous liquid which is purified by column chromatography (silica gel 230-400 mesh, mobile phase n-hexane:ethyl acetate 75:25) to get 4-[(4-decyl-2,3-difluorobenzyl)methylamino]cyclohexane-1,1-dicarboxylic acid diethyl ester.

### Preparation of 4-[(4-decyl-2,3-difluorobenzyl)methylamino]cyclohexane-1,1-dicarboxylic acid ethyl ester

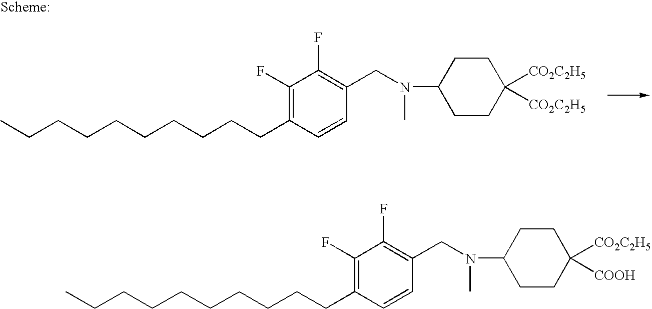

To a stirred solution of 4-[(4-decyl-2,3-difluorobenzyl)(methyl)amino]cyclohexane-1,1-dicarboxylic acid diethyl ester (0.5 g, 0.00095 mole) in ethanol (5 mL), a solution of sodium hydroxide (0.15 g, 0.0038 mole) in water (5 mL) is added at room temperature. The reaction mixture is heated under reflux for 5 hours, concentrated under reduced pressure and the residue is treated with water (2 mL). The aqueous solution is acidified to pH 6-7 by drop wise addition of dil. HCl. solution is evaporated to obtain sticky solid, purified by column chromatography (silica gel 230-400 mesh, dichloromethane:methanol 85:15) to get 4-[(4-decyl-2,3-difluoro-benzyl)(methyl)amino]-cyclohexane-1,1-dicarboxylic acid ethyl ester.

### Preparation of 4-[(4-decyl-2,3-difluoro-benzyl)methylamino]cyclohexane-1,1-dicarboxylic acid

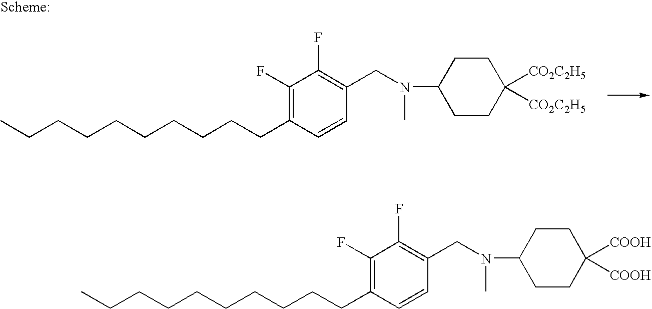

To a stirred solution of 4-[(4-decyl-2,3-difluorobenzyl)methyl-amino]cyclohexane-1,1-dicarboxylic acid ethyl ester (0.64 g, 0.0012 mole) in ethanol (5 mL), a solution of potassium hydroxide (0.82 g, 0.014 mole) in water (4 mL) is added at room temperature. The reaction mixture is heated at 90° C. for overnight, concentrated under reduced pressure and the residue is treated with water (2 mL) and acidified to pH ˜6-7. Aqueous layer is extracted with tetrahydrofuran (2×20 mL), combined organic layer is washed with brine (1×10 mL) and dried over anhydrous sodium sulphate. Removal of solvent under reduced pressure furnished solid, which is washed with diethyl ether (15 mL) to get 4-[(4-decyl-2,3-difluorobenzyl)methyl-amino]cyclohexane-1,1-dicarboxylic acid

Similar way following compounds are also prepared:

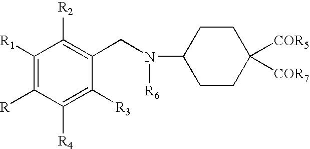

| | R | R1 | R2 | R3 | R4 | R5 | R6 | R7 | |
|---|---|---|---|---|---|---|---|---|---|
| 133 | C10H21 | F | F | H | H | OEt | CH3 | OEt | 524.2 |
| 134 | C9H19 | F | F | H | H | OEt | CH3 | OEt | 510.2 |
| 135 | C9H19 | F | F | H | H | OH | H | OH | 439.6 |
| 136 | C9H19 | F | F | H | H | OEt | CH3 | OH | 482.2 |
| 137 | C10H21 | F | F | H | H | OEt | CH3 | OH | 496.2 |
| 138 | C10H21 | F | F | H | H | OH | H | OH | 454.2 |
| 139 | C9H19 | F | F | H | H | OH | CH3 | OH | 454.2 |
| 140 | C10H21 | F | F | H | H | OH | CH3 | OH | 468.2 |

### Preparation of 1-(4-decyl benzyl)azetidine-3,3-dicarboxylic acid diethyl ester

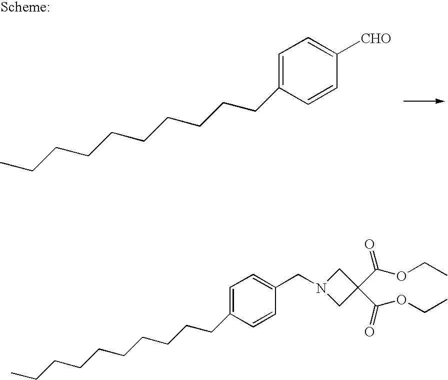

To a stirred solution of azetidine-3,3-dicarboxylic acid diethyl ester (0.8 g, 0.00397 mole) (Synth. Commun., 2003, 33, 3347-3353) methanol (10 mL), a solution of 4-decylbenzaldehyde (0.97 g, 0.00397 mole) in ethanol (5 mL) is added at room temperature. The reaction mixture is stirred for 1.5 h at room temperature. Glacial acetic acid (1 mL) is added to the mixture, cooled to 5-100 C and treated with sodium cyanoborohydride (0.29 g, 0.00476 mole) is added. The reaction mixture is brought to room temperature and is allowed to stir at room temperature for 3 hours. The mixture is concentrated under reduced pressure and the residue is treated with D.M. water (10 mL) and extracted with ethyl acetate (2×20 mL). Combined organic layer is washed with brine (1×10 mL) and dried over anhydrous sodium sulphate. Removal of solvent furnished a viscous liquid which is purified by column chromatography (silica gel 230-400 mesh, mobile phase n-hexane:ethyl acetate 7:3) to get 1-(4-decyl benzyl)azetidine-3,3-dicarboxylic acid diethyl ester.

### Preparation of 1-(4-decylbenzyl)azetidine-3,3-dicarboxylic acid

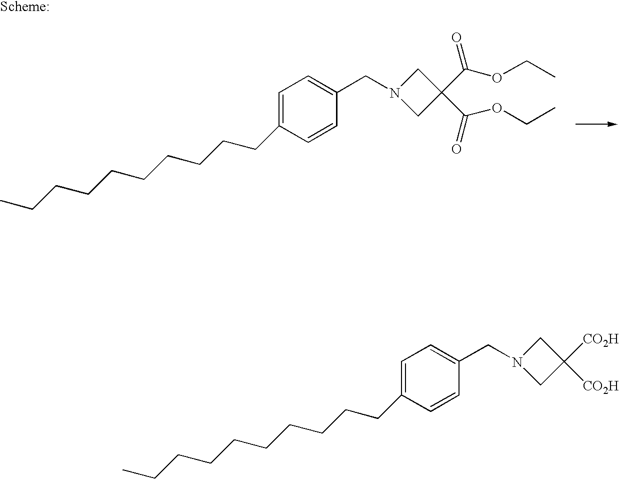

To a stirred solution of 1-(4-decylbenzyl)azetidine-3,3-dicarboxylic acid diethyl ester (0.95 g, 0.0022 mole) in ethanol (10 mL), a solution of sodium hydroxide (0.44 g, 0.0110 mole) in water (6 mL) is added at room temperature. The reaction mixture is heated under reflux for 5 hours. The reaction mixture is concentrated under reduced pressure and the residue is treated with D.M. water (2 mL). The aqueous solution is acidified to pH ˜2 by drop wise addition of dil. HCl. White solid mass is filtered and washed with diethyl ether:methanol (8:2) furnished 1-(4-decylbenzyl)azetidine-3,3-dicarboxylic acid.

| Ex. | | | | | | | | |
|---|---|---|---|---|---|---|---|---|
| 141 | 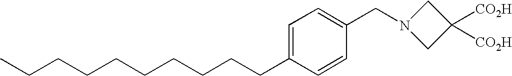 | | | | | | | 376.5 |
| | Compound | | | | | | | |
| | 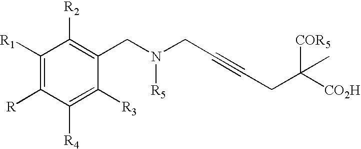 | | | | | | | |
| Ex. | R | R1 | R2 | R3 | R4 | R5 | R6 | MS (ESI+) |
| 142 | C10H21O | Cl | H | H | H | OH | H | 466.4, |
| | | | | | | | | 468.3 |
| 143 | H15C7n-4-Ph | H | H | H | F | OH | H | 468.3 |
| 144 | C9H19 | H | H | H | H | OH | 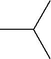 | 444.4 |
| 145 | C9H19 | H | H | H | H | OH | 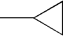 | 442.3 |
| 146 | H13C6n-4-Ph | H | H | H | F | OH | CH3 | 468.2 |
| 147 | C9H19 | H | H | H | H | OH | CH3 | 416.3 |
| 148 | C9H19 | F | F | H | H | OH | CH3 | 452.3 |
| 149 | C9H19 | H | H | H | H | OH | C2H5 | 430.4 |
| 150 | C9H19 | F | F | H | H | OH | H | 438.4 |
| 151 | C10H21 | F | F | H | H | OH | H | 452.5 |
| 152 | C10H21 | H | H | H | H | OH | CH3 | 430.2 |
| 153 | C9H19O | Cl | H | H | H | OH | CH3 | 466.1, |
| | | | | | | | | 468.1 |

| | | |
|---|---|---|
| 154 | 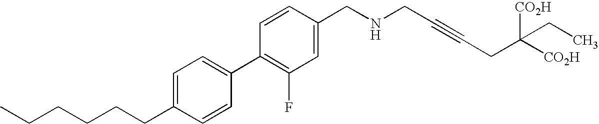 | 468.2 |
| 155 | 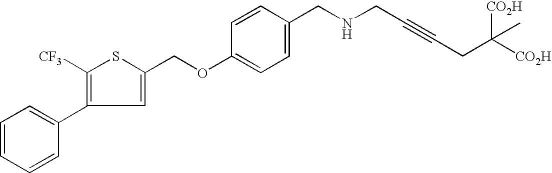 | 530.0 |

### EXAMPLE 32

### 3-[(2-Fluoro-4′-heptylbiphenyl-4-ylmethyl)amino]cyclopentane-1,1-dicarboxylic acid

13C NMR: (CDCl3+TFA; 100.61 MHz; ppm)

14.65 (q); 23.29 (t); 29.16 (t); 29.80 (t); 30.02 (t); 32.01 (t); 32.46 (t); 33.11 (t); 36.31 (t); 37.17 (t); 50.66 (t); 58.63 (d); 59.73 (s); 117.89 [d (d), JC—F=24.35 Hz]; 126.23 (d); 129.30 (4d); 130.54 [s (d), JC—F=8.02 Hz]; 131.66 [s (d), JC—F=13.54 Hz]; 132.26 (d); 132.38 [s (d), JC—F=2.42 Hz]; 144.12 (s); 160.39 [s (d), JC—F=249.74 Hz]; 174.81 (s); 176.12 (s)

### EXAMPLE 33

### 1-Carbamoyl-3-(4-nonylbenzylamino)cyclopentanecarboxylic acid—Isomer B

13C NMR: (TFA+CDCl3; 100.61 MHz; ppm)

14.64 (q); 23.29 (t); 29.95 (t); 29.99 (2t), 30.07 (t); 30.16 (t); 31.86 (t); 32.52 (t); 36.26 (t); 36.28 (t); 37.47 (t); 51.08 (t); 59.06 (d); 59.50 (s); 127.23 (s): 130.16 (2d); 130.19 (2d); 146.24 (s); 175.15 (s); 177.61 (br s)

### EXAMPLE 34

### 3-(3-Chloro-4-decyloxybenzylamino)cyclopentane-1,1-dicarboxylic acid

13C NMR: (CDCl3+TFA); 100.61 MHz; ppm)

14.71 (q); 23.33 (t); 26.53 (t); 29.12 (t); 29.64 (t); 29.98 (t); 30.02 (t); 30.20 (t); 30.22 (t); 32.56 (t); 33.05 (t); 37.24 (t); 50.52 (t); 58.27 (d); 59.78 (s); 70.03 (t); 114.21 (d); 122.84 (s); 124.07 (s); 130.22 (d); 132.11 (d); 156.53 (s); 174.98 (s); 176.00 (s).

### EXAMPLE 35

### 3-(3-Chloro-4-octyloxybenzylamino)cyclopentane-1,1-dicarboxylic acid

13C NMR: (CDCl3+TFA); 100.61 MHz; ppm)

14.63 (q); 23.26 (t); 26.48 (t); 29.17 (t); 29.57 (t); 29.81 (t); 29.91 (t); 32.42 (t); 33.08 (t); 37.18 (t); 50.56 (t); 58.30 (d); 59.71 (s); 70.06 (t); 114.29 (d); 122.70 (s); 124.18 (s); 130.13 (d); 132.02 (d); 156.60 (s); 174.83 (s); 176.01 (s).

### EXAMPLE 36

### 1-Carbamoyl-3-(4-nonylbenzylamino)cyclopentanecarboxylic acid—Isomer A

13C NMR: (TFA+CDCl3; 100.61 MHz; ppm)

14.71 (q); 23.33 (t); 29.68 (t); 29.98 (2t); 30.11 (t); 30.19 (t); 31.86 (t); 32.55 (t); 35.72 (t); 36.31 (t); 36.91 (t); 51.62 (t); 58.27 (d); 59.92 (s); 126.76 (s); 130.20 (2d); 130.29 (2d); 146.50 (s); 175.92 (s); 176.90 (s)

### EXAMPLE 37

### 3-[Cyclopropyl-(4-nonylbenzyl)amino]cyclopentane-1,1-dicarboxylic acid

13C NMR: (DMSO-d6; 100.61 MHz; ppm)

5.18 (2t); 13.95 (q); 22.11 (t); 27.30 (t); 28.64 (t); 28.70 (t); 28.86 (t); 28.98 (t); 30.43 (t); 30.82 (t); 31.29 (t); 34.86 (t); 35.35 (t); 35.51 (d); 56.64 (t); 57.28 (s); 64.35 (d); 127.34 (s); 128.30 (2d); 131.63 (2d); 143.40 (s); 172.39 (s); 173.06 (s)

### EXAMPLE 38

### 3-[(2-Fluoro-4′-heptylbiphenyl-4-ylmethyl)methylamino]cyclopentane-1,1-dicarboxylic acid

13C NMR: (CDCl3+TFA; 100.61 MHz; ppm)

13.94 (q); 22.09 (t); 26.67 (t); 28.55 (t); 28.68 (t); 30.74 (t); 30.87 (t); 31.25 (t); 34.84 (2t); 36.74 (q); 56.42 (t); 57.88 (s); 64.82 (d); 119.06 (d, J=23.98 Hz); 127.93 (d, J=2.67 Hz); 128.61 (2d); 128.65 (d); 128.67 (d); 129.05 (s, J=13.10 Hz); 130.78 (d, J=3.48 Hz); 131.47 (s, J=8.18 Hz); 131.69 (s); 142.53 (s); 158.67 (s, J=246.59 Hz); 172.22 (s); 172.89 (s)

### EXAMPLE 39

### 3-(2,3-Difluoro-4-nonylbenzylamino)cyclopentane-1,1-dicarboxylic acid

13C NMR: (CDCl3+TFA; 100.61 MHz; ppm)

14.69 (q); 23.33 (t); 29.50 (t); 29.92 (t); 29.96 (2t); 29.98 (t); 30.13 (t); 30.36 (t); 32.54 (t); 33.57 (t); 37.23 (t); 45.15 (t); 59.03 (d); 59.80 (s); 116.25 [s, (d, J=11.42 Hz)]; 126.21 (d); 126.85 (d); 136.56 [s, (d, J=12.84 Hz)]; 149.52 [s, (dd, J1=254.38 Hz, J2=16.99 Hz)]; 150.11 [s, (dd, J1=245.07 Hz, J2=10.33 Hz)]; 176.30 (s); 177.31 (s).

### EXAMPLE 40

### 3-(2,3-Difluoro-4-nonylbenzylamino)cyclopentane-1,1-dicarboxylic acid ethyl ester

13C NMR: (CDCl3+TFA; 100.61 MHz; ppm)

14.35 (q); 14.74 (q); 23.33 (t); 29.52 (t); 29.59 (t); 29.92 (t); 29.96 (t); 29.98 (t); 30.12 (t); 30.37 (t); 32.53 (t); 33.73 (t); 37.21 (t); 44.73 (t); 58.80 (d); 59.98 (s); 63.73 (t); 116.53 [s, (d, J=11.41 Hz)]; 126.35 (d); 126.78 (d); 136.21 [s, (d J=12.74 Hz)]; 149.44 [s, (dd, J1=250.60 Hz, J2=12.74 Hz)]; 150.12 [s, (dd, J1=252.93 Hz, J2=16.67 Hz]; 171.30 (s); 177.66 (s).

### EXAMPLE 41

### 3-(4-Nonyl-benzylamino)cyclopentane-1,1-dicarboxylic acid isobutyl ester

13C NMR: (CDCl3+TFA; 100.61 MHz; ppm)

14.71 (q); 19.30 (2q); 23.35 (t); 28.27 (d); 29.72 (t); 29.96 (t); 30.01 (t); 30.13 (t); 30.19 (t); 31.85 (t); 32.57 (t); 33.49 (t); 36.32 (t); 37.36 (t); 52.11 (t); 58.69 (d); 59.86 (s); 74.06 (t); 127.68 (s); 130.12 (2d); 130.38 (2d); 146.74 (s); 172.03 (s); 177.94 (s).

### EXAMPLE 42

### 3-[Methyl-(4-nonylbenzyl)amino]cyclopentane-1,1-dicarboxylic acid ethyl ester

13C NMR: (DMSO-d6; 100.61 MHz; ppm)

13.81 (q); 13.94 (q); 22.08 (t); 26.57 (t); 28.68 (2t); 28.85 (t); 28.94 (t); 30.67 (t); 30.79 (t); 31.26 (t); 34.84 (2t); 36.58 (q); 57.13 (t); 57.98 (s); 61.31 (t); 64.04 (d); 127.25 (s); 128.55 (2d); 131.34 (2d); 143.68 (s); 171.24 (s); 171.68 (s)

### EXAMPLE 43

### 3-(4-Nonylbenzylamino)cyclopentane-1,1-dicarboxylic acid methyl ester

13C NMR: (CDCl3; 100.61 MHz; ppm)

14.62 (q); 23.17 (t); 29.36 (t); 29.82 (t); 29.92 (t); 29.99 (t); 30.05 (t); 31.81 (t); 32.37 (t); 32.95 (t); 36.23 (t); 37.11 (t); 50.46 (t); 53.63 (q); 56.89 (d); 59.69 (s); 127.69 (s); 129.66 (2d); 131.03 (2d); 145.04 (s); 171.94 (s); 175.01 (s)

### EXAMPLE 44

### 3-(4-Nonylbenzylamino)cyclopentane-1,1-dicarboxylic acid benzyl ester

13C NMR: (CDCl3+TFA; 100.61 MHz; ppm)

14.69 (q); 23.35 (t); 29.71 (t); 29.96 (t); 30.00 (t); 30.12 (t); 30.19 (t); 31.84 (t); 32.57 (t); 33.49 (t); 36.31 (t); 37.32 (t); 52.10 (t); 58.65 (d); 59.90 (br s); 69.65 (t); 126.55 (s); 128.99 (2d); 129.48 (2d); 129.68 (d); 130.14 (2d); 130.37 (2d); 134.88 (s); 146.74 (s); 171.55 (s); 178.24 (br s)

### EXAMPLE 45

### 3-[(2-Fluoro-4′-octylbiphenyl-4-ylmethyl)amino]cyclopentane-1,1-dicarboxylic acid ethyl ester

13C NMR: (CDCl₃; 100.61 MHz; ppm)

14.55 (q); 14.72 (q); 23.28 (t); 29.51 (t); 29.88 (t); 30.03 (t); 30.09 (t); 32.01 (t); 32.50 (t); 32.99 (t); 36.31 (t); 37.04 (t); 49.91 (t); 57.40 (d); 59.82 (s); 62.77 (t); 118.88 [d (d, JC—F=24.03 Hz)]; 127.28 (d); 129.22 (2d); 129.32 [2d (d, JC—F=2.42 Hz)]; 130.82 [s (d, JC—F=13.07 Hz); 131.32 [s (d, JC—F=7.63 Hz)]; 131.97 (s); 132.46 (d); 143.75 (s); 160.11 [s (d, JC—F=249.81 Hz)]; 171.38 (s); 175.25 (s)

### EXAMPLE 46

### 3-[(2-Fluoro-4′-octylbiphenyl-4-ylmethyl)amino]cyclopentane-1,1-dicarboxylic acid

13C NMR: (CDCl3+TFA; 100.61 MHz; ppm)

14.72 (q); 23.37 (t); 29.63 (t); 29.97 (t); 30.09 (t); 30.17 (t); 32.09 (t); 32.59 (t); 33.52 (t); 36.41 (t); 37.37 (t); 51.35 (t); 58.97 (d); 60.02 (s); 118.08 [d (d, JC—F=24.38 Hz)]; 126.26 (d); 129.43 (4d); 129.88 [s (d, JC—F=7.59 Hz); 132.13 (s); 132.28 [s (d, JC—F=13.27 Hz)]; 132.69 [d (d, JC—F=3.17 Hz)]; 144.33 (s); 160.53 [s (d, JC—F=251.02 Hz)]; 176.69 (s); 177.70 (s)

### EXAMPLE 47

### 3-[Cyclopropyl-(2-fluoro-4′-heptylbiphenyl-4-ylmethyl)amino]cyclopentane-1,1-dicarboxylic acid

13C NMR: (DMSO-d6; 100.61 MHz; ppm)

6.23 (2t); 13.97 (q); 22.13 (t); 28.08 (t); 28.59 (t); 28.71 (t), 30.85 (t); 30.93 (t); 31.29 (t); 34.87 (t); 35.34 (d); 36.01 (t); 56.17 (t); 57.35 (s); 64.16 (d); 117.68 (d); 126.73 (d); 127.63 (s); 128.57 (2d); 128.61 (2d, J=2.90 Hz); 130.25 [d (d, JC—F=3.26 Hz); 132.07 (2s); 142.23 (s); 158.73 [s (d, JC—F=246.06 Hz)]; 173.03 (s); 173.46 (s)

### EXAMPLE 48

### 3-(4-Nonylbenzylamino)cyclopentane-1,1-dicarboxylic acid isopropylester

13C NMR: (CDCl3+TFA; 100.61 MHz; ppm)

14.70 (q); 21.78 (q); 21.79 (q); 23.33 (t); 29.78 (t); 29.95 (t); 29.99 (t); 30.11 (t); 30.17 (t); 31.84 (t); 32.56 (t); 33.57 (t); 36.30 (t); 37.36 (t); 52.09 (t); 58.71 (d); 59.79 (s); 72.59 (d); 126.64 (s); 130.18 (2d); 130.85 (2d); 146.67 (s); 171.58 (s); 178.05 (s).

### EXAMPLE 49

### 3-(2,3-Difluoro-4-nonylbenzylamino)-1-methylcarbamoylcyclopentane carboxylic acid—Isomer A

13C NMR (CDCl3+TFA); 100.61 MHz; ppm)

14.58 (q); 23.37 (t); 28.24 (q); 29.56 (t); 29.64 (t); 30.01 (t); 30.03 (2t); 30.19 (t); 30.42 (t); 32.61 (t); 35.85 (t); 37.31 (t); 45.47 (t); 59.40 (d); 59.94 (s); 116.12 [s, (d, J=11.37 Hz)]; 126.38 (d); 126.93 (d); 136.91 [s, (d, J=12.85 Hz)]; 149.68 [s, (dd, J1=242.17 Hz, J2=4.26 Hz)]; 150.27 [s, (dd, J1=247.51 Hz, J2=12.65 Hz)]; 173.11 (s); 178.75 (s)

### EXAMPLE 50

### 3-(2,3-Difluoro-4-nonylbenzylamino)-1-methylcarbamoylcyclopentane carboxylic acid—Isomer B

13C NMR: (CDCl3+TFA); 100.61 MHz; ppm)

14.59 (q); 23.35 (t); 28.32 (q); 29.53 (t); 30.01 (3t); 30.17 (t); 30.38 (2t); 32.59 (t); 37.37 (t); 37.83 (t); 44.96 (t); 59.44 (d); 59.93 (s); 116.28 [s, (d, J=10.48 Hz)]; 126.37 (d); 126.90 (d); 136.09 [s, (d, J=12.37 Hz)]; 149.64 [s, (dd, J1=250.66 Hz, J2=13.58 Hz)]; 150.26 [s, (dd, J1=250.88 Hz, J2=16.55 Hz)]; 174.80 (s); 177.67 (s)

### EXAMPLE 51

### 3-(4-Nonylbenzylamino)-1-propylcarbamoylcyclopentanecarboxylic acid—Isomer A

13C NMR: (CDCl3+TFA; 100.61 MHz; ppm)

11.48 (q); 14.66 (q); 22.57 (t); 23.36 (t); 29.64 (t); 30.02 (2t); 30.14 (t); 30.22 (t); 31.88 (t); 32.59 (t); 35.81 (t); 36.33 (t); 37.27 (t); 43.58 (t); 52.05 (t); 58.61 (d); 59.89 (s); 126.59 (s); 130.34 (2d); 130.44 (2d); 146.78 (s); 172.36 (s); 178.39 (s)

### EXAMPLE 52

### 3-(4-Nonylbenzylamino)-1-propylcarbamoylcyclopentanecarboxylic acid—Isomer B

13C NMR: (CDCl3+TFA; 100.61 MHz; ppm)

11.40 (q); 14.61 (q); 22.49 (t); 23.35 (t); 30.02 (2t); 30.15 (t); 30.22 (t); 30.62 (t); 31.88 (t); 32.60 (t); 36.33 (t); 37.49 (t); 37.92 (t); 43.75 (t); 51.92 (t); 59.34 (d); 59.50 (s); 126.77 (s); 130.36 (2d); 130.43 (2d); 146.76 (s); 174.03 (s); 177.99 (s)

### EXAMPLE 53

### 3-3(4-Decyl-2,3-difluorobenzylamino)cyclopentane-1,1-dicarboxylic acid

13C NMR: (CDCl3+TFA; 100.61 MHz; ppm)

14.64 (q); 23.39 (t); 29.56 (t); 29.61 (t); 29.99 (t); 30.03 (t); 30.05 (t); 30.23 (t); 30.32 (t); 30.41 (t); 32.63 (t); 33.43 (t); 37.40 (t); 45.57 (t); 59.31 (d); 59.90 (s); 116.07 [s (d), JC—F=11.58 Hz]; 126.1 [d (d), JC—F=2.67 Hz); 127.02 [d (d), JC—F=3.9 Hz); 137.01 [s (d), JC—F=12.93 Hz); 149.67 [s (dd), J1 C—F=249.05 Hz, J2 C—F=11.27 Hz); 150.2 [s (dd), J1 C—F=249.11 Hz, J2 C—F=14.24 Hz]; 176.81 (s); 177.79 (s)

### EXAMPLE 54

### 3-(4-Decylbenzylamino)-1-methylcarbamoylcyclopentanecarboxylic acid—Isomer A

13C NMR: (CDCl3+TFA; 100.61 MHz; ppm)

14.62 (q); 23.40 (t); 28.30 (q); 29.78 (t); 30.06 (t); 30.08 (t); 30.18 (t); 30.30 (t); 30.36 (t); 31.92 (t); 32.66 (t); 35.83 (t); 36.37 (t); 37.35 (t); 52.41 (t); 58.91 (d); 59.89 (s); 126.50 (s); 130.38 (2d); 130.48 (2d); 147.07 (s); 173.18 (s); 178.66 (s)

### EXAMPLE 55

### 3-(4-Decylbenzylamino)-1-methylcarbamoylcyclopentanecarboxylic acid—Isomer B

13C NMR: (CDCl3+TFA; 100.61 MHz; ppm)

14.60 (q); 23.35 (t); 28.62 (q); 30.02 (2t); 30.13 (t); 30.25 (t); 30.30 (t); 30.78 (t); 31.87 (t); 32.60 (t); 36.32 (t); 37.50 (t); 38.16 (t); 52.15 (t); 59.33 (d); 59.62 (s); 126.73 (s); 130.38 (2d); 130.56 (2d); 146.77 (s); 174.89 (s); 177.78 (s)

### EXAMPLE 56

### 1-Ethylcarbamoyl-3-(4-nonylbenzylamino)cyclopentanecarboxylic acid—Isomer A

13C NMR: (CDCl3+TFA); 100.61 MHz; ppm)

14.01 (q); 14.58 (q); 23.40 (t); 29.70 (t); 30.07 (t); 30.09 (t); 30.20 (t); 30.28 (t); 31.94 (t); 32.66 (t); 35.81 (t); 36.38 (t); 37.11 (t); 37.29 (t); 52.35 (t); 58.92 (s); 59.95 (d); 126.55 (s); 130.40 (2d); 130.48 (2d); 147.11 (s); 172.34 (s); 178.92 (s).

### EXAMPLE 57

### 1-Ethylcarbamoyl-3-(4-nonylbenzylamino)cyclopentanecarboxylic acid—Isomer B

13C NMR: (CDCl3+TFA); 100.61 MHz; ppm)

14.09 (q); 14.69 (q); 23.34 (t); 30.00 (2t); 30.12 (t); 30.19 (t); 30.39 (t); 31.86 (t); 32.57 (t); 36.31 (t); 36.90 (t); 37.19 (t); 37.76 (t); 51.54 (t); 59.20 (s); 59.25 (d); 126.88 (s); 130.23 (2d); 130.42 (2d); 146.44 (s); 173.76 (s); 177.44 (s).

### EXAMPLE 58

### 1-Isopropylcarbamoyl-3-(4-nonylbenzylamino)cyclopentanecarboxylic acid—Isomer A

Mass (Es+mode):

(M+1)+=431.4

### EXAMPLE 59

### 1-Isopropylcarbamoyl-3-(4-nonylbenzylamino)cyclopentanecarboxylic acid—Isomer B

Mass (Es+mode):

(M+1)+=431.4

### EXAMPLE 60

### 3-(4-Decyl-2,3-difluorobenzylamino)-1-methylcarbamoyl cyclopentanecarboxylic acid—Isomer A

13C NMR: (CDCl3+TFA; 100.61 MHz; ppm)

14.58 (q); 23.40 (t); 28.28 (q); 29.58 (t); 29.72 (t); 30.03 (t); 30.06 (t); 30.07 (t); 30.26 (t); 30.34 (t); 30.44 (t); 32.66 (t); 35.93 (t); 37.37 (t); 45.64 (t); 59.49 (d); 59.96 (s); 116.11 [s (d), JC—F=11.28 Hz); 126.35 (d); 126.99 (d); 137.02 [s (d), JC—F=12.87 Hz); 149.73 [s (dd), J1 C—F=247.23 Hz, J2 C—F=9.56 Hz); 150.3 [s (dd), J1 C—F=248.49 Hz, J2 C—F=13.06 Hz); 173.13 (s); 178.81 (s)

### EXAMPLE 61

### 3-[(2,3-Difluoro-4-nonylbenzyl)methylamino]-1-methylcarbamoylcyclopentane carboxy-lic acid—Isomer A

13C NMR: (CDCl3+TFA); 100.61 MHz; ppm)

14.72 (q); 23.33 (t); 28.12 (q); 28.85 (t); 29.14 (t); 29.56 (t); 29.97 (2t); 30.12 (t); 30.33 (t); 30.98 (t); 34.91 (t); 36.65 (t); 39.23 (q); 53.05 (t); 58.78 (s); 67.28 (d); 114.52 [s, (d, J=3.81 Hz); 126.83 (d); 127.61 (d); 136.81 [s, (d, J=12.88 Hz)]; 149.69 [s, dd, J1=249.24 Hz, J2=12.04 Hz]; 150.55 [s, (dd, J1=250.25 Hz, J2=13.81 Hz)]; 173.37 (s); 176.47 (s).

Extra set of peaks are as follows:

28.16 (q); 36.82 (t); 39.49 (q); 53.21 (t); 58.95 (s); 67.64 (d); 114.73 (d); 176.61 (s).

### EXAMPLE 62

### 3-[(2,3-Difluoro-4-nonylbenzyl)methylamino]-1-methylcarbamoylcyclopentane carboxylic acid—Isomer B

Mass (ES+mode):

(M+1)+=453.4

### EXAMPLE 63

### 3-[(4-Decylbenzyl)methylamino]cyclopentane-1,1-dicarboxylic acid

13C NMR: (TFA+CDCl3; 100.61 MHz; ppm)

14.73 (q); 23.35 (t); 28.94 (t); 29.99 (2t); 30.11 (t); 30.23 (t); 30.28 (t); 31.83 (t); 32.58 (t); 32.86 (t); 36.34 (t); 36.44 (t); 39.06 (q); 58.98 (s); 60.81 (t); 66.57 (d); 124.81 (s); 130.48 (2d); 131.38 (2d); 147.21 (s); 176.36 (2s) Each peaks marked in bold showed another peak of same intensity

### EXAMPLE 64

### 3-(4-Nonylbenzylamino)cyclopentane-1,1-dicarboxylic acid butyl ester

13C NMR: (CDCl3+TFA; 100.61 MHz; ppm)

14.05 (q); 14.71 (q); 19.55 (t); 23.35 (t); 29.72 (t); 29.96 (t); 30.01 (t); 30.12 (t); 30.19 (t); 30.82 (t); 31.85 (t); 32.57 (t); 33.52 (t); 36.31 (t); 37.34 (t); 53.00 (t); 58.68 (d); 59.81 (s); 68.15 (t); 126.60 (s); 130.13 (2d); 130.37 (2d); 146.74 (s); 172.13 (s); 177.99 (s).

### EXAMPLE 65

### 3-(4-Nonylbenzylaminocyclopentane-1,1-dicarboxylic acid propyl ester

13C NMR: (CDCl3+TFA; 100.61 MHz; ppm)

10.94 (q); 14.76 (q); 22.44 (t); 23.32 (t); 29.90 (t); 29.97 (t); 30.05 (t); 30.13 (t); 30.19 (t); 31.96 (t); 32.53 (t); 33.27 (t); 36.36 (t); 37.11 (t); 50.22 (t); 56.70 (d); 60.06 (s); 68.22 (t); 127.65 (s); 129.86 (2d); 131.08 (2d); 145.36 (s); 171.17 (s); 176.02 (s)

### EXAMPLE 66

### 1-(3,4-Difluorophenyl carbamoyl)-3-(4-nonylbenzylamino)cyclopentane carboxylic acid

13C NMR: (CDCl3+TFA); 100.61 MHz; ppm)

14.66 (q); 23.36 (t); 30.02 (2t); 30.15 (t); 30.22 (t); 30.49 (t); 31.90 (t); 32.60 (t); 36.34 (t); 37.42 (t); 37.84 (t); 51.77 (t); 59.55 (s); 59.97 (d); 112.83 (d, J=21.32 Hz); 118.42 (d, J=18.50 Hz); 119.11 (dd, J1=6.23 Hz, J2=3.74 Hz); 126.83 (s); 130.34 (2d); 130.26 (2d); 132.37 [s, (dd, J1=8.27 Hz, J2=3.40 Hz); 146.69 (s); 149.39 [s, (dd, J1=249.10 Hz, J2=12.57 Hz); 150.89 [s, (dd, J1=249.35 Hz, J2=13.49 Hz); 172.29 (s); 177.90 (s).

### EXAMPLE 67

### 3-[(2,3-Difluoro-4-nonylbenzyl)methylamino]cyclopentane-1,1-dicarboxylic acid

13C NMR: (TFA+CDCl3; 100.61 MHz; ppm)

14.60 (q); 23.34 (t); 28.48 (t); 29.60 (t); 29.99 (2t); 30.16 (t); 30.37 (t); 32.57 (t); 32.86 (t); 35.86 (t); 36.45 (t); 39.34 (q); 53.97 (t); 59.10 (s); 67.18 (d); 114.33 [s (d), JC—F=13.90 Hz]; 127.14 (d); 127.18 (d); 137.59 [s (d), JC—F=12.95 Hz); 149.84 [s (dd), J1 C—F=248.87 Hz, J2 C—F=10.78 Hz], 150.56 [s (dd), J1 C—F=250.132 Hz, J2 C—F=14.39 Hz]; 176.69 (2s)

Extra set of peaks

28.93 (t); 32.94 (t); 39.59 (q); 54.29 (t); 59.25 (s); 67.39 (d); 114.45 (s); 137.62 (s)

### EXAMPLE 68

### 3-(4-Decyl-2,3-difluorobenzylamino)cyclopentane-1,1-dicarboxylic acid isopropyl ester

13C NMR: (TFA+CDCl3; 100.61 MHz; ppm)

14.62 (q); 21.61 (q); 21.64 (q); 23.39 (t); 29.56 (t); 29.69 (t); 29.99 (t); 30.04 (t); 30.05 (t); 30.23 (t); 30.33 (t); 30.42 (t); 32.64 (t); 33.77 (t); 37.44 (t); 45.55 (t); 59.42 (d); 60.13 (s); 73.56 (d); 110.90 (s); 126.16 [d (d), JC—F=2.96 Hz]; 126.99 (d); 136.95 [s (d), JC—F=12.95 Hz]; 149.68 [s (dd), J1 C—F=248.914 Hz, J2 C—F=11.03 Hz]; 150.22 [s (dd), J1 C—F=248.998 Hz, J2 C—F=14.16 Hz]; 172.26 (s); 178.86 (s)

### EXAMPLE 69

### 3-(4-Decylbenzylamino)cyclopentane-1,1-dicarboxylic acid isopropyl ester

13C NMR: (TFA+CDCl3; 100.61 MHz; ppm)

14.70 (q); 21.72 (q); 21.75 (q); 23.37 (t); 29.74 (t); 29.98 (t); 30.02 (t); 30.13 (t); 30.24 (t); 30.31 (t); 31.87 (t); 32.60 (t); 33.57 (t); 36.32 (t); 37.35 (t); 52.15 (t); 58.75 (d); 59.87 (s); 72.88 (d); 126.58 (s); 130.14 (2d); 130.40 (2d); 146.80 (s); 171.81 (s); 206.01 (s)

### EXAMPLE 70

### 3-[(2-Fluoro-4′-heptylbiphenyl-4-ylmethyl)amino]-1-methylcarbamoyl-cyclopentanecarboxylic acid—Isomer A

13C NMR: (CDCl3+TFA; 100.61 MHz; ppm)

14.58 (q); 23.42 (t); 28.25 (q); 29.74 (t); 29.98 (t); 30.13 (t); 32.19 (t); 32.62 (t); 35.81 (t); 36.50 (t); 37.32 (t); 51.67 (t); 59.28 (d); 59.97 (s); 118.30 (d, J=24.57 Hz); 126.43 (d, J=3.20 Hz); 129.51 (d); 129.56 (3d); 129.81 [s, (d, J=7.67 Hz)]; 132.23 (d); 132.66 [s, (d, J=13.34 Hz)]; 132.86 [s, (d, J=3.88 Hz,)]; 144.60 (s); 160.75 (s, J=251.03 Hz); 173.22 (s); 178.63 (s).

### EXAMPLE 71

### 3-[(2-Fluoro-4′-heptylbiphenyl-4-ylmethyl)amino]-1-methylcarbamoylcyclopentane-carboxylic acid—Isomer B

13C NMR: (CDCl3+TFA; 100.61 MHz; ppm)

14.70 (q); 23.34 (t); 28.15 (q); 29.86 (t); 30.02 (t); 30.53 (t); 32.08 (t); 32.50 (t); 36.39 (t); 37.33 (t); 37.77 (t); 50.71 (t); 59.08 (d); 59.73 (s); 118.22 (d, J=24.44 Hz); 126.43 (d, J=3.21 Hz); 129.40 (3d); 129.42 (d); 130.35 [s, (d, J=7.77 Hz)]; 131.96 [s, (d, J=13.34 Hz)]; 132.27 (d); 132.53 [s, (d, J=3.93 Hz)]; 144.21 (s); 160.49 [s, (d, J=251.52 Hz)]; 174.90 (s); 176.67 (s).

### EXAMPLE 72

### 3-(4-Decylbenzylamino)cyclopentane-1,1-dicarboxylic acid enantiomer A

13C NMR: (TFA+CDCl3; 100.61 MHz; ppm)

14.66 (q); 23.38 (t); 29.68 (t); 30.00 (t); 30.04 (t); 30.14 (t); 30.27 (t); 30.33 (t); 31.87 (t); 32.62 (t); 33.42 (t); 36.34 (t); 37.39 (t); 52.32 (t); 58.75 (d); 59.78 (s); 126.43 (s); 130.12 (2d); 130.48 (2d); 147.03 (s); 176.71 (s); 177.53 (s)

### EXAMPLE 73

### 3-(4-Decylbenzylamino)cyclopentane-1,1-dicarboxylic acid enantiomer B

13C NMR: (TFA+CDCl3; 100.61 MHz; ppm)

14.65 (q); 23.39 (t); 29.67 (t); 30.01 (t); 30.05 (t); 30.15 (t); 30.28 (t); 30.34 (t); 31.89 (t); 32.63 (t); 33.37 (t); 36.34 (t); 37.38 (t); 52.36 (t); 58.75 (d); 59.76 (s); 126.41 (s); 130.12 (2d); 130.50 (2d); 147.09 (s); 176.82 (s); 177.59 (s)

### EXAMPLE 74

### 3-[(3-Chloro-4-decyloxy-benzyl)(methyl)amino]-cyclopentane-1,1-dicarboxylic acid

13C NMR: (DMSO-d6); 100.61 MHz; ppm)

13.91 (q); 22.07 (t); 25.39 (t); 28.45 (2t); 28.66 (2t); 28.90 (t); 28.96 (t); 31.28 (t); 34.42 (t); 36.99 (t); 37.40 (q); 55.42 (t); 56.98 (s); 66.52 (d); 68.61 (t); 113.57 (d); 121.26 (s); 126.00 (s); 130.47 (d); 131.63 (d); 154.11 (s); 176.43 (s); 176.82 (s)

### EXAMPLE 75

### 3-[(3-Chloro-4-nonylloxybenzyl)methyl-amino]cyclopentane-1,1-dicarboxylic acid

13C NMR: (DMSO-d6); 100.61 MHz; ppm)

13.97 (q); 22.14 (t); 25.44 (t); 28.44 (t); 28.50 (t); 28.66 (t); 28.71 (t); 28.98 (t); 31.30 (t); 34.40 (t); 34.44 (t); 36.88 (q); 55.47 (s); 56.93 (t); 66.56 (d); 68.65 (t); 113.60 (d); 121.31 (d); 125.46 (s); 131.85 (d); 139.22 (s); 154.26 (s); 176.43 (s); 176.80 (s).

### EXAMPLE 76

### 1-Dimethylcarbamoyl-3-(4-nonylbenzylamino)cyclopentanecarboxylic acid

13C NMR: (CDCl3+TFA; 100.61 MHz; ppm)

14.63 (q); 23.36 (t); 30.02 (2t); 30.14 (2t); 30.22 (t); 31.89 (t); 32.60 (t); 33.43 (t); 36.34 (t); 38.55 (q); 39.25 (q); 40.19 (t); 52.13 (t); 58.18 (d); 59.90 (s); 126.70 (s); 130.35 (2d); 130.52 (2d); 146.68 (s); 172.66 (s); 177.71 (s)

### EXAMPLE 77

### 1-Ethylcarbamoyl-3-(4-octylbenzylamino)cyclopentanecarboxylic acid—Isomer A

13C NMR: (CDCl3+TFA); 100.61 MHz; ppm)

14.16 (q); 14.61 (q); 23.34 (t); 29.81 (t); 29.92 (t); 30 01 (t); 30.10 (t); 31.88 (t); 32.58 (t); 35.99 (t); 36.34 (t); 37.03 (t; 37.34 (t); 52.38 (t); 58.86 (d); 59.89 (s); 126.52 (s); 130.39 (2d); 130.45 (2d); 146.97 (s); 172.28 (s); 178.59 (s).

### EXAMPLE 78

### 1-Ethylcarbamoyl-3-(4-octylbenzylamino)cyclopentanecarboxylic acid—Isomer B

13C NMR: (CDCl3+TFA); 100.61 MHz; ppm)

14.01 (q); 14.63 (q); 23.33 (t); 29.91 (t); 30.00 (t); 30.56 (t); 31.86 (t); 32.56 (t); 36.32 (t); 37.04 (t); 37.36 (t); 37.61 (t); 37.83 (t); 51.78 (t); 59.26 (d); 59.44 (s); 126.77 (s); 130.35 (4d); 146.69 (s); 173.86 (s); 177.67 (s).

Another set of peak observed at—

14.14 (q); 30.09 (t); 30.60 (t); 36.90 (t); 37.67 (t); 51.68 (t); 59.05 (d); 126.83 (s); 146.61 (s); 173.86 (s); 174.24 (s).

### EXAMPLE 79

### 1-Methylcarbamoyl-3-(4-octylbenzylamino)cyclopentanecarboxylic acid—Isomer A

13C NMR: (CDCl3+TFA); 100.61 MHz; ppm)

14.63 (q); 23.34 (t); 28.20 (q); 29.63 (t); 29.92 (t); 30.01 (t); 30.09 (t); 31.87 (t); 32.57 (t); 35.68 (t); 36.33 (t); 37.26 (t); 52.03 (t); 58.63 (d); 59.81 (s); 126.60 (s); 130.35 (2d); 130.40 (2d); 146.79 (s); 173.13 (s); 178.14 (s).

### EXAMPLE 80

### 1-Methylcarbamoyl-3-(4-octylbenzylamino)cyclopentanecarboxylic acid—Isomer B

13C NMR: (CDCl3+TFA); 100.61 MHz; ppm)

14.63 (q); 23.33 (t); 28.28 (q); 29.91 (t); 30.01 (t); 30.09 (t); 30.43 (t); 31.87 (t); 32.57 (t); 36.33 (t); 37.12 (t); 37.80 (t); 51.69 (t); 59.33 (d+s); 126.79 (s); 130.32 (2d); 130.38 (2d); 146.67 (s); 174.75 (s); 177.55 (s).

### EXAMPLE 81

### 3-(4-octylbenzylamino)cyclopentane-1,1-dicarboxylic acid isopropyl ester

13C NMR: (CDCl3+TFA); 100.61 MHz; ppm)

14.69 (q); 21.77 (q); 21.78 (q); 23.33 (t); 29.72 (t); 29.89 (t); 29.99 (t); 30.08 (t); 31.86 (t); 32.55 (t); 33.49 (t); 36.32 (t); 37.35 (t); 51.76 (t); 58.40 (d); 59.82 (s); 72.64 (d); 126.71 (s); 130.26 (2d); 130.49 (2d); 146.54 (s); 171.78 (s); 177.91 (s).

### EXAMPLE 82

### 3-[(4-Decyl-2,3-difluorobenzyl)methylamino]cyclopentane-1,1-dicarboxylic acid

13C NMR: (TFA+CDCl3; 100.61 MHz; ppm)

14.67 (q); 23.35 (t); 28.40 (t); 29.58 (t); 29.98 (2t); 30.18 (t); 30.26 (t); 30.35, (t); 32.58 (t); 32.74 (t); 35.89 (t); 36.42 (t); 39.13 (q); 53.57 (t); 58.98 (s); 67.03 (d); 114.39 [s (d), JC—F=11.53 Hz]; 127.06 (d); 127.24 (d); 137.30 [s (d), JC—F=12.92 Hz); 149.76 [s (dd), J1 C—F=249.30 Hz, J2 C—F=11.79 Hz], 150.56 [s (dd), J1 C—F=250.42 Hz, J2 C—F=14.18 Hz]; 176.29 (s); 176.43 (s)

Extra set of peaks

28.84 (t); 32.76 (t); 39.36 (q); 53.84 (t); 59.13 (s); 67.25 (d); 114.51 (s); 137.34 (s)

### EXAMPLE 83

### 3-(4-Decyl-2,3-difluorobenzylamino)cyclopentane-1,1-dicarboxylic acid enantiomer B

13C NMR: (CDCl3+TFA; 100.61 MHz; ppm)

14.70 (q); 23.03 (t); 29.49 (t); 29.57 (t); 29.94 (t); 29.98 (2t); 30.18 (t); 30.26 (t); 30.36 (t); 32.57 (t); 33.63 (t); 37.24 (t); 45.13 (t); 59.01 (d); 59.81 (s); 116.28 [s (d), JC—F=11.37 Hz]; 126.26 (d); 126.83 (d); 136.51 [s (d), JC—F=12.74 Hz); 149.52 [s (dd), J1 C—F=251.28 Hz, J2 C—F=14.28 Hz); 150.13 [s (dd), J1 C—F=254.93 Hz, J2 C—F=20.26 Hz]; 176.09 (s); 177.21 (s)

### EXAMPLE 84

### 3-(4-Decyl-2,3-difluorobenzylamino)cyclopentane-1,1-dicarboxylic acid enantiomer A

13C NMR: (CDCl3+TFA; 100.61 MHz; ppm)

14.70 (q); 23.33 (t); 29.34 (t); 29.46 (t); 29.98 (2t); 30.09 (t); 30.19 (t); 30.25 (t); 30.38 (t); 32.56 (t); 33.55 (t); 37.27 (t); 44.65 (t); 58.90 (d); 59.77 (s); 116.61 [s (d), JC—F=10.34 Hz]; 126.30 (d); 126.66 (d); 136.08 [s (d), JC—F=12.23 Hz); 149.47 [s (dd), J1 C—F=250.10 Hz, J2 C—F=13.65 Hz); 150.08 [s (dd), J1 C—F=251.262 Hz, J2 C—F=16.2 Hz]; 175.10 (s); 176.58 (s)

### EXAMPLE 85

### 3-[(2-Fluoro-4′-heptylbiphenyl-4-ylmethyl)amino]cyclopentane-1,1-dicarboxylic acid enantiomer B

13C NMR: (CDCl3+TFA; 100.61 MHz; ppm)

14.67 (q); 23.35 (t); 29.62 (t); 29.87 (t); 30.03 (t); 32.09 (t); 32.52 (t); 33.60 (t); 36.40 (t); 37.36 (t); 51.31 (t); 59.07 (d); 59.64 (s); 118.04 [d (d), JC—F=24.48 Hz]; 126.23 [d (d), JC—F=2.9 Hz]; 129.45 (4d); 129.91 [s (d), JC—F=7.69 Hz]; 132.16 (s); 132.32 [s (d), JC—F=13.34 Hz]; 132.72 [d (d), JC—F=3.65 Hz]; 144.38 (s); 160.57 [s (d), JC—F=250.66 Hz]; 176.12 (s); 177.17 (s)

### EXAMPLE 86

### 3-(4-Decylbenzylamino)cyclopentane-1,1-dicarboxylic acid ethyl ester—Isomer A

13C NMR: (CDCl3+TFA; 50.33 MHz; ppm)

14.17 (q); 14.67 (q); 23.37 (t); 29.74 (t); 29.99 (t); 30.02 (t); 30.13 (t); 30.25 (t); 30.31 (t); 31.86 (t); 32.61 (t); 33.61 (t); 36.32 (t); 37.42 (t); 52.20 (t); 58.74 (d); 59.83 (s); 64.54 (t); 126.56 (s); 130.19 (2d); 130.42 (2d); 146.86 (s); 172.34 (s); 177.99 (s)

### EXAMPLE 87

### 3-(4-Decylbenzylamino)cyclopentane-1,1-dicarboxylic acid ethyl ester—Isomer B

13C NMR: (CDCl3+TFA; 50.33 MHz; ppm)

14.18 (q); 14.65 (q); 23.35 (t); 29.91 (t); 29.99 (t); 30.01 (t); 30.12 (t); 30.24 (t); 30.30 (t); 31.87 (t); 32.59 (t); 33.29 (t); 36.32 (t); 37.76 (t); 52.36 (t); 58.89 (d); 60.22 (s); 65.01 (t); 126.68 (s); 130.40 (2d); 130.51 (2d); 146.76 (s); 174.00 (s); 177.04 (s)

### EXAMPLE 88

### 3-[(4-Decylbenzyl)methylamino]cyclopentane-1,1-dicarboxylic acid ethyl ester

13C NMR: (CDCl3); 100.61 MHz; ppm)

14.74 (q); 14.83 (q); 23.30 (t); 29.94 (2t); 30.10 (t); 30.18 (t); 30.24 (t); 30.57 (t); 31.92 (t); 32.51 (t); 33.84 (t); 36.28 (t); 36.95 (t); 39.00 (q); 58.63 (t); 60.67 (s); 61.65 (t); 66.33 (d); 127.81 (s); 129.58 (2d); 131.71 (2d); 144.88 (s); 174.67 (s); 176.73 (s)

### EXAMPLE 89

### 3-[(4-Decyl-2,3-difluorobenzyl)methylamino]cyclopentane-1,1-dicarboxylic acid enan-tiomer B

13C NMR: (DMSO-d6+CDCl3; 100.61 MHz; ppm)

13.74 (q); 22.02 (t); 28.00 (t); 28.62 (2t); 28.67 (2t); 28.89 (2t); 29.35 (t); 31.22 (t); 33.05 (t); 37.16 (t); 38.35 (q); 51.21 (t); 56.65 (s); 65.87 (d); 121.68 (s); 124.63 (d); 125.93 (d); 131.17 [Is (d), JC—F=12.15 Hz]; 148.04 [s (dd), J1 C—F=243.32 Hz, J2 C—F=9.84 Hz]; 148.67 [s (dd), J1 C—F=244.25 Hz, J2 C—F=9.42 Hz]; 174.99 (s); 175.30 (s)

### EXAMPLE 90

### 3-[(4-Decyl-2,3-difluoro-benzyl)(methyl)amino]cyclopentane-1,1-dicarboxylic acid ethyl ester enantiomer B

13C NMR: (DMSO-d6+CDCl3; 100.61 MHz; ppm)

13.81 (q); 13.87 (q); 22.13 (t); 28.04 (t); 28.62 (t); 28.73 (t); 28.78 (t); 28.97 (t); 29.00 (2t); 29.52 (t); 31.33 (t); 31.58 (t); 37.41 (t); 38.98 (q); 51.69 (t); 58.49 (s); 60.65 (t); 64.75 (d); 124.40 (d); 124.6 [s (d); JC—F=11.51 Hz]; 125.37 (d); 129.96 [s (d); JC—F=12.93 Hz]; 148.14 [s (dd), J1 C—F=244.263 Hz, J2 C—F=12.82 Hz]; 148.58 [s (dd), J1 C—F=246.08 Hz, J2 C—F=13.20 Hz]; 172.13 (s); 173.37 (s)

### EXAMPLE 91

### 3-[(4-Decyl-2,3-difluorobenzyl)methyl-amino]cyclopentane-1,1-dicarboxylic acid enan-tiomer A

13C NMR: (DMSO-d6+CDCl3; 100.61 MHz; ppm)

14.16 (q); 22.63 (t); 28.82 (t); 29.21 (t); 29.25 (2t); 29.29 (t); 29.47 (t); 29.53 (t); 29.77 (t); 31.83 (t); 35.87 (t); 37.97 (t); 37.97 (q); 50.98 (t); 55.38 (s); 68.05 (d); 116.64 [s (d), JC—F=9.97 Hz]; 125.85 (d); 126.78 (d); 134.41 [s (d), JC—F=12.96 Hz]; 148.84 [s (dd), J1 C—F=247.35 Hz, J2 C—F=12.36 Hz]; 149.70 [s (dd), J1 C—F=249.58 Hz, J2 C—F=13.85 Hz]; 177.40 (s); 179.57 (s)

### EXAMPLE 92

### 3-[(4-Decyl-2,3-difluorobenzyl)methylamino]cyclopentane-1,1-dicarboxylic acid ethyl ester enantiomer A

13C NMR: (DMSO-d6+CDCl3; 50.327 MHz; ppm)

14.02 (2q); 22.49 (t); 28.56 (t); 29.05 (t); 29.11 (t); 29.18 (t); 29.33 (t); 29.39 (t); 29.63 (t); 29.77 (t); 31.70 (t); 32.32 (t); 37.09 (t); 38.59 (q); 51.09 (t); 59.39 (s); 60.96 (t); 65.61 (d); 121.38 [s (d), JC—F=11.35 Hz]; 124.76 [d (br t)]; 126.03 (d); 131.71 [s (d), JC—F=13.00 Hz]; 148.61 [s (dd), J1 C—F=245.46 Hz, J2 C—F=12.59 Hz]; 149.33 [s (dd), J1 C—F=247.23 Hz, J2 C—F=13.09 Hz]; 172.92 (s); 174.91 (s)

### EXAMPLE 93

### 3-(4-Decyl-2,6-difluorobenzylamino)cyclopentane-1,1-dicarboxylic acid

13C NMR: (CDCl3+TFA; 100.61 MHz; ppm)

14.73 (q); 23.35 (t); 29.42 (t); 29.79 (t); 29.98 (t); 30.01 (t); 30.17 (t); 30.25 (t); 31.17 (t); 32.56 (t); 33.69 (t); 36.45 (t); 37.27 (t); 38.94 (t); 59.01 (d); 59.73 (s); 103.46 [s (t), J C—F=19.15 Hz]; 112.40 [2d (d), J C—F=23.21 Hz]; 151.02 [s (t), J C—F=9.49 Hz]; 161.90 [2s (d), J1 C—F=250.47 Hz, J2 C—F=7.35 Hz]; 175.76 (s); 177.05 (s)

### EXAMPLE 94

### 3-[(4-Decyl-2,6-difluorobenzyl)methylamino]cyclopentane-1,1-dicarboxylic acid

13C NMR: (DMSO-d6; 100.61 MHz; ppm)

13.94 (q); 22.09 (t); 27.7 (t); 28.52 (t); 28.68 (t); 28.76 (t); 28.95 (t); 28.97 (t); 30.13 (t); 31.29 (t); 31.43 (t); 34.67 (t); 35.89 (t); 38.25 (q); 45.5 (t); 57.68 (s); 65.21 (d); 105.22 (br s); 111.58 [2d (d), JC—F=22.49 Hz]; 148.29 (br s); 161.16 [s (d), JC—F=249.29 Hz]; 161.25 [s (d), JC—F=248.91 Hz]; 172.98 (s); 173.31 (s)

### EXAMPLE 95

### 3-[(4-Decyl-2,3-difluorobenzyl)ethoxycarbonylamino]cyclopentane-1,1-dicarboxylic acid

13C NMR: (CDCl3+TFA); 100.61 MHz; ppm)

14.74 (q); 14.92 (q); 23.35 (t); 29.37 (2t); 29.30 (2t); 30.05 (t); 30.23 (t); 30.27 (t); 30.63 (t); 32.58 (t); 32.76 (t); 36.93 (t); 42.97 (t); 58.45 (s); 58.87 (d); 63.73 (t); 122.61 (d); 124.87 [s, (d, J=11.18 Hz)]; 125.48 (d); 131.92 [s, (d, J=12.90 Hz)]; 148.89 [s, (dd), J1=250.41 Hz, J2=17.29 Hz)]; 149.56 [s, (dd), J1=248.79 Hz, J2=14.70 Hz)]; 158.17 (s); 177.92 (2s).

### EXAMPLE 96

### 3-[(4-Decyl-2,3-difluorobenzyl)ethoxycarbonylamino]cyclopentane-1,1-dicarboxylic acid ethyl ester—Isomer A

13C NMR: (CDCl3+TFA); 100.61 MHz; ppm)

14.59 (q); 14.77 (q); 15.19 (q); 23.34 (t); 29.36 (t); 29.96 (t); 29.98 (t); 30.06 (t); 30.22 (t); 30.26 (t); 30.37 (t); 30.67 (t); 32.56 (t); 32.70 (t); 37.02 (t); 42.54 (t); 58.13 (s); 58.89 (d); 62.42 (t); 62.60 (t); 122.73 (d); 125.23 (d); 125.97 [s, (d, J=11.06 Hz)]; 131.24 [s, (d, J=12.85 Hz)]; 148.81 [s, (dd, J1=249.80 Hz, J2=17.07 Hz)]; 149.45 [s, (dd, J1=248.08 Hz, J2=14.43 Hz)]; 157.15 (s); 172.39 (s); 177.35 (s).

### EXAMPLE 97

### 3-[(4-Decyl-2,3-difluorobenzyl)ethoxycarbonylamino]cyclopentane-1,1-dicarboxylic acid ethyl ester—Isomer B

13C NMR: (CDCl3+TFA); 100.61 MHz; ppm)

14.56 (q); 14.77 (q); 15.19 (q); 23.34 (t); 29.36 (t); 29.95 (t); 29.98 (t); 30.05 (t); 30.22 (t); 30.26 (t); 30.37 (t); 30.68 (t); 32.56 (2t); 37.09 (t); 41.74 (t); 57.78 (s); 58.74 (d); 62.50 (t); 62.62 (t); 122.56 (d); 125.24 (d); 126.06 [s, (d, J=10.9 Hz)]; 131.18 [s, (d, J=12.70 Hz)]; 148.70 [s, (dd, J1=248.68 Hz, J2=16.24 Hz)]; 149.44 [s, (dd, J1=247.76 Hz, J2=14.16 Hz)]; 157.29 (s); 172.53 (s); 177.09 (s).

### EXAMPLE 98

### 3-[(4-Decyl-2,3-difluorobenzyl)methoxycarbonylamino]cyclopentane-1,1-dicarboxylic acid

13C NMR: (CDCl3+TFA); 100.61 MHz; ppm)

14.74 (q); 23.35 (t); 29.37 (2t); 29.99 (2t); 30.05 (t); 30.22 (t); 30.27 (t); 30.63 (t); 32.57 (t); 32.75 (t); 36.93 (t); 42.97 (t); 54.42 (q); 58.50 (d); 58.86 (s); 122.47 (d); 124.73 [s, (d, J=11.14 Hz)]; 125.52 [d, (t, J=4.04 Hz)]; 131.96 [s, (d, J=13.00 Hz)]; 148.83 [s, (dd, J1=250.81 Hz, J2=17.93 Hz]; 149.54 [s, (dd), J1=249.36 Hz, J2=15.05 Hz)]; 158.60 (s); 177.82 (2s).

### EXAMPLE 99

### 3-[(4-Decyl-2,3-difluorobenzyl)methoxycarbonylamino]cyclopentane-1,1-dicarboxylic acid ethyl ester

13C NMR: (CDCl3+TFA); 100.61 MHz; ppm)

14.57 (q); 14.78 (q); 23.34 (t); 29.35 (2t); 29.96 (t); 29.98 (t); 30.05 (t); 30.21 (t); 30.26 (t); 30.66 (t); 32.55 (t); 32.63 (t); 36.82 (t); 42.47 (t); 53.57 (q); 58.11 (d); 58.85 (s); 62.61 (t); 122.53 (d); 125.27 [d, (t, J=4.14 Hz)]; 125.78 [s, (d, J=11.22 Hz]); 131.28 [s, (d, J=12.93 Hz)]; 147.48 [s, (dd, J1=250.70 Hz, J2=14.48 Hz)]; 149.30 [s, (dd), J1=248.29 Hz, J2=14.45 Hz)]; 157.61 (s); 172.31 (s); 177.54 (s).

### EXAMPLE 100

### 2-Methyl-2-[4-(4-nonylbenzylamino)but-2-enyl]malonic acid

13C NMR: (CDCl3); 100.61 MHz; δ ppm)

14.76 (q); 23.33 (t); 27.20 (q); 29.97 (2t); 30.12 (t); 30.19 (t); 31.94 (t); 32.53 (t); 36.32 (2t); 42.03 (t); 51.23 (t); 53.63 (s); 120.80 (d); 128.10 (s); 129.91 (2d); 130.67 (2d); 138.22 (d); 145.53 (s); 179.59 (2s).

### EXAMPLE 101

### 3-[2-(4-Octyl-phenyl)ethylamino]cyclopentane-1,1-dicarboxylic acid

13C NMR: (TFA+CDCl3; 100.61 MHz; ppm)

14.66 (q); 23.32 (t); 29.49 (t); 29.92 (t); 30.01 (t); 30.11 (t); 32.06 (t); 32.56 (t); 32.74 (t); 33.39 (t); 36.17 (t); 37.55 (t); 49.99 (t); 59.49 (d); 59.94 (s); 129.15 (2d); 129.99 (2d); 132.28 (s); 143.72 (s); 176.13 (s); 176.51 (s)

### EXAMPLE 102

### 1-(1-Carboxy-2-phenylethylcarbamoyl)-3-(4-nonylbenzylamino)cyclopentane-carboxylic acid

13C NMR: (CDCl3+TFA; 100.61 MHz; ppm)

14.50 (q); 23.43 (t); 30.11 (t); 30.13 (t); 30.25 (t); 30.33 (t); 30.69 (t); 32.02 (t); 32.71 (t); 36.45 (t); 37.23 (t); 37.41 (t); 37.58 (t); 38.09 (t); 37.66 (t); 37.77 (t); 51.98 (t); 52.16 (t); 55.07 (d); 55.20 (d); 59.48 (d); 59.74 (d); 59.61 (s); 126.71 (s); 128.81 (2d); 128.95 (2d); 129.87 (2d); 129.90 (2d); 129.97 (2d); 130.03 (2d); 130.16 (2d); 130.24 (2d); 130.64 (d); 134.97 (s); 135.31 (s); 147.26 (s); 173.78 (s); 173.85 (s); 177.59 (s); 177.68 (s); 178.31 (s); 178.58 (s)

Extra peaks marked with are observed because of the presence of chiral centres in the molecule

### EXAMPLE 103

### 3-{4-[5-(3-Chloro-4-ethoxyphenyl)pentyloxy]benzylamino}cyclopentane-1,1-dicarbo-xylic acid

13C NMR: (CDCl3+TFA; 100.61 MHz, ppm)

15.34 (q); 26.10 (t); 29.15 (t); 29.56 (t); 31.76 (t); 33.05 (t); 35.24 (t); 37.21 (t); 51.02 (t); 58.03 (d); 59.74 (s); 65.67 (t); 68.68 (t); 114.43 (d); 115.89 (d); 122.02 (s); 123.20 (s); 128.12 (2d); 130.64 (2d); 131.79 (d); 136.45 (s); 153.04 (s); 160.89 (s); 174.84 (s); 175.96 (s).

### EXAMPLE 104

### 3-[4-(4-Phenyl-5-trifluoromethylthiophen-2-ylmethoxy)benzylamino]cyclopentane-1,1-dicarboxylic acid enantiomer B

13C NMR: (TFA+CDCl3; 100.61 MHz; ppm)

29.75 (t); 33.61 (t); 37.54 (t); 51.94 (t); 58.71 (d); 59.84 (s); 65.51 (t); 116.65 (2d); 122.42 (s); 123.13 [s (q), JC—F=269.951 Hz]; 126.42 [s (q), JC—F=36.83 Hz); 129.06 (3d); 129.10 (d); 129.35 (2d); 130.84 (d); 132.11 (d); 134.93 (s); 140.99 (s); 144.85 [s (q), JC—F=2.3 Hz]; 160.22 (s); 175.22 (s); 176.46 (s)

### EXAMPLE 105

### 3-{Methyl-[4-(4-phenyl-5-trifluoromethylthiophen-2-ylmethoxy)benzyl]amino}-cyclopentane-1,1-dicarboxylic acid

13C NMR: (DMSO-d6; 100.61 MHz; ppm)

27.81 (t); 34.57 (t); 36.44 (t); 36.71 (q); 55.18 (t); 57.07 (s); 63.92 (t); 65.93 (d); 114.90 (2d); 122.52 [s (q), JC—F=269.64 Hz]; 123.08 [s (q), JC—F=35.81 Hz); 124.32 (s); 128.41 (2d); 128.60 (3d); 130.77 [d (d), J=2.60 Hz]; 132.50 (2d); 133.69 (s); 142.53 (s); 144.07 [s (q), JC—F=2.9 Hz]; 158.00 (s); 176.81 (2s)

### EXAMPLE 106

### 3-[(2-Fluoro-4′-heptylbiphenyl-4-ylmethyl)amino]cyclobutane-1,1-dicarboxylic acid

13C NMR: (CDCl3+TFA; 100.61 MHz; ppm)

14.66 (q); 23.40 (t); 29.93 (t); 30.07 (t); 32.14 (t); 32.57 (t); 33.81 (2t); 36.45 (t); 47.98 (s); 48.18 (d); 50.48 (t); 118.05 (d, J=24.51 Hz); 126.15 (d, J=3.24 Hz); 129.35 (s); 129.46 (d); 129.49 (d); 129.52 (2d); 132.06 (s); 132.69 [s, (d, J=13.29 Hz)]; 132.90 (d, J=3.94 Hz); 144.56 (s); 160.70 [s, (d, J=251.52 Hz)]; 175.90 (s); 176.39 (s).

### EXAMPLE 107

### 3-(4-Nonylbenzylamino)cyclobutane-1,1-dicarboxylic acid

13C NMR: (CDCl3+TFA; 100.61 MHz; ppm)

14.71 (q); 23.34 (t); 29.99 (2t); 30.11 (t); 30.20 (t); 31.86 (t); 32.55 (t); 33.72 (2t); 36.30 (t); 47.77 (d); 47.86 (s); 50.76 (t); 126.48 (s); 130.10 (2d); 130.30 (2d); 146.59 (s); 175.28 (s); 175.67 (s)

### EXAMPLE 108

### 3-(4-Decylbenzylamino)cyclobutane-1,1-dicarboxylic acid

13C NMR: (CDCl3+TFA; 100.61 MHz; ppm)

14.68 (q); 23.38 (t); 29.99 (t); 30.04 (t); 30.14 (t); 30.27 (t); 30.32 (t); 31.86 (t); 32.62 (t); 33.81 (2t); 36.33 (t); 47.90 (s); 47.90 (d); 51.17 (t); 126.18 (s); 130.10 (2d); 130.49 (2d); 147.03 (s); 175.72 (s); 176.30 (s)

### EXAMPLE 109

### 3-[(4-Decylbenzyl)methylamino]cyclobutane-1,1-dicarboxylic acid

13C NMR: (CDCl3+TFA; 100.61 MHz; ppm)

14.72 (q); 23.35 (t); 29.99 (2t); 30.11 (t); 30.23 (t); 30.28 (t); 31.83 (t); 32.58 (t); 33.31 (t); 33.99 (t); 36.34 (t); 37.54 (q); 46.30 (s); 55.50 (d); 58.84 (t); 124.23 (s); 130.53 (2d); 131.36 (2d); 147.31 (s); 174.99 (s); 175.72 (s)

### EXAMPLE 110

### 3-(4-Decyl-2,3-difluorobenzylamino)cyclobutane-1,1-dicarboxylic acid

13C NMR: (CDCl3+TFA; 100.61 MHz; ppm)

14.61 (q); 23.40 (t); 29.59 (t); 30.01 (t); 30.05 (t); 30.07 (t); 30.25 (t); 30.34 (t); 30.42 (t); 32.65 (t); 33.77 (2t); 44.50 (t); 48.02 (s); 48.44 (d); 115.78 [s (d), JC—F=11.57 Hz); 126.09 [d (d), JC—F=2.73 Hz]; 127.09 (d); 137.23 [s (d), JC—F=12.94 Hz]; 149.72 [s (dd), J1 C—F=249.58 Hz, J2 C—F=11.48 Hz]; 150.24 [s (dd), J1 C—F=249.45 Hz, J2 C—F=14.46 Hz]; 176.10 (s); 176.52 (s)

### EXAMPLE 111

### 3-[(2-Fluoro-4′-heptylbiphenyl-4-ylmethyl)amino]-1-methylcarbamoylcyclo-butanecarboxylic acid—Isomer A

13C NMR: (TFA+CDCl3; 100.61 MHz; ppm)

14.66 (q); 23.38 (t); 28.23 (q); 29.91 (t); 30.06 (t); 32.12 (t); 32.55 (t); 34.61 (2t); 36.44 (t); 47.92 (s); 48.57 (d); 50.48 (t); 118.22 [d (d), JC—F=24.57 Hz]; 126.39 (d); 129.48 (4d); 129.61 [s (d), JC—F=7.73 Hz]; 132.13 (d); 132.47 [s (d), JC—F=13.22 Hz]; 132.75 [s (d), JC—F=3.37 Hz]; 144.44 (s); 160.64 [s (d), JC—F=253.16 Hz]; 172.05 (s); 176.85 (s)

### EXAMPLE 112

### 3-[(2-Fluoro-4′-heptylbiphenyl-4-ylmethyl)amino]-1-methylcarbamoylcyclo-butanecarboxylic acid—Isomer B

13C NMR: (TFA+CDCl₃; 100.61 MHz; ppm)

14.71 (q); 23.34 (t); 28.18 (q); 29.86 (t); 30.02 (t); 30.07 (t); 32.06 (t); 32.50 (t); 34.80 (t); 36.38 (t); 47.60 (s); 49.12 (d); 50.19 (t); 118.4 [d (d), JC—F=24.19 Hz]; 126.63 (d); 129.40 (4d); 130.01 [s (d), JC—F=7.22 Hz]; 132.01 [s (d), JC—F=13.03 Hz]; 132.17 (d); 132.55 (s); 144.24 (s); 160.48 [s (d), JC—F=254.56 Hz]; 173.36 (s); 176.28 (s)

### EXAMPLE 113

### 3-[(4-Decylbenzyl)methylamino]cyclobutane-1,1-dicarboxylic acid ethyl ester

13C NMR: (CDCl3+TFA; 100.61 MHz; ppm)

14.33 (q); 14.75 (q); 23.34 (t); 29.99 (2t); 30.11 (t); 30.23 (t); 30.28 (t); 31.84 (t); 32.56 (t); 33.07 (t); 33.78 (t); 36.34 (t); 37.18 (q); 46.54 (s); 54.88 (d); 58.04 (t); 63.60 (t); 124.65 (s); 130.30 (2d); 131.60 (2d); 146.76 (s); 171.29 (s); ˜173 (s)

### EXAMPLE 114

### 3-[(4-Decyl-2,3-difluorobenzyl)methylamino]cyclobutane-1,1-dicarboxylic acid

13C NMR: (CDCl3+TFA; 100.61 MHz; ppm)

14.69 (q); 23.33 (t); 29.55 (2t); 29.97 (2t); 30.18 (t); 30.25 (t); 30.33 (t); 32.56 (t); 33.30 (t); 33.86 (t); 37.42 (q); 46.09 (s); 51.26 (t); 55.95 (d); 114.25 [s (d), JC—F=11.37 Hz]; 126.96 (d); 127.23 (d); 137.09 [s (d), JC—F=12.88 Hz]; 149.69 [s (dd), J1 C—F=249.01 Hz, J2 C—F=11.68 Hz]; 150.49 [s (dd), J1 C—F=250.17 Hz, J2 C—F=13.38 Hz]; 174.50 (s); 174.73 (s)

### EXAMPLE 115

### 3-[(4-Decyl-2,3-difluorobenzyl)methylamino]cyclobutane-1,1-dicarboxylic acid ethyl ester

13C NMR: (CDCl3+TFA; 100.61 MHz; ppm)

14.30 (q); 14.75 (q); 23.34 (t); 29.58 (2t); 29.97 (2t); 30.17 (t); 30.25 (t); 30.36 (t); 32.56 (t); 33.14 (t); 33.69 (t); 37.36 (q); 46.34 (s); 50.61 (t); 55.40 (d); 63.84 (t); 114.23 [s (d), JC—F=11.31 Hz]; 126.94 (d); 127.50 (d); 136.82 [s (d), JC—F=12.79 Hz]; 149.66 [s (dd), J1 C—F=249.06 Hz, J2 C—F=12.12 Hz]; 150.60 [s (dd), J1 C—F=250.04 Hz, J2 C—F=14.14 Hz]; 171.21 (s); 173.91 (s)

### EXAMPLE 116

### 3-[4-(4-Phenyl-5-trifluoromethylthiophen-2-ylmethoxy)benzylamino]cyclobutane-1,1-dicarboxylic acid

13C NMR: (TFA+CDCl3; 100.61 MHz; ppm)

33.83 (2t); 47.89 (s); 47.89 (d); 50.79 (t); 65.48 (t); 116.70 (2d); 122.09 (s); 123.14 [s (q), JC—F=270.01 Hz]; 126.46 [s (q), JC—F=36.75 Hz); 129.06 (3d); 129.11 (d); 129.34 (d); 129.35 (d); 130.87 (d); 131.98 (d); 134.93 (s); 140.93 (s); 144.88 [s (q), JC—F=2.84 Hz]; 160.30 (s); 175.56 (s); 176.02 (s)

### EXAMPLE 117

### 1-(4-Decylbenzyl)piperidine-4,4-dicarboxylic acid

13C NMR: (TFA+CDCl3; 100.61 MHz; ppm)

14.65 (q); 23.36 (t); 28.21 (2t); 30.02 (2t); 30.12 (t); 30.26 (t); 30.31 (t); 31.85 (t); 32.61 (t); 36.35 (t); 50.34 (t); 51.71 (s); 62.55 (2t); 124.56 (s); 130.50 (2d); 131.16 (2d); 147.48 (s); 174.15 (s); 174.60 (s)

### EXAMPLE 118

### 1-(4-Nonylbenzyl)piperidine-4,4-dicarboxylic acid

13C NMR: (CDCl3+TFA); 100.61 MHz; ppm)

14.64 (q); 23.35 (t); 28.20 (2t); 30.00 (2t); 30.12 (t); 30.20 (t); 31.85 (t); 32.58 (t); 36.35 (t); 50.28 (2t); 51.72 (s); 62.52 (t); 124.55 (s); 131.21 (2d); 131.50 (2d); 147.49 (s); 174.38 (s); 174.80 (s).

### EXAMPLE 119

### 1-(4-Decyl-2,3-difluorobenzyl)piperidine-4,4-dicarboxylic acid

13C NMR: (TFA+CDCl3; 100.61 MHz; ppm)

14.62 (q); 23.35 (t); 28.20 (2t); 29.56 (t); 29.98 (2t); 30.01 (t); 30.19 (t); 30.27 (t); 30.35 (t); 32.59 (t); 50.64 (2t); 51.54 (s); 55.12 (t); 114.25 [s (d), JC—F=11.42 Hz]; 127.05 (2d); 137.49 [s (dd), JC—F=12.92 Hz]; 149.75 [s (dd), J1 C—F=249.43 Hz, J2 C—F=11.57 Hz]; 150.44 [s (d), J1 C—F=250.37 Hz, J2 C—F=13.92 Hz]; 174.08 (s); 174.50 (s)

### EXAMPLE 120

### 1-(4-Octylbenzyl)piperidine-4,4-dicarboxylic acid

13C NMR: (CDCl3+TFA); 100.61 MHz; ppm)

14.60 (q); 23.34 (t); 28.23 (2t); 29.92 (t); 30.01 (t); 30.10 (t); 31.86 (t); 32.58 (t); 36.37 (t); 50.33 (2t); 51.76 (s); 62.61 (t); 124.52 (s); 130.54 (2d); 131.26 (2d); 147.57 (s); 174.64 (s); 175.03 (s).

### EXAMPLE 121

### 1-(3-Chloro-4-nonyloxybenzyl)piperidine-4,4-dicarboxylic acid

13C NMR: (CDCl3+TFA); 100.61 MHz; ppm)

14.73 (q); 23.33 (t); 26.53 (t); 28.30 (2t); 29.60 (t); 29.91 (t); 29.98 (t); 30.15 (t); 32.53 (t); 50.17 (2t); 51.68 (s); 61.15 (t); 70.06 (t); 114.17 (d); 120.56 (s); 124.38 (s); 131.23 (d); 133.09 (d); 157.09 (s); 173.17 (s); 174.03 (s).

### EXAMPLE 122

### 1-(2-Fluoro-4′-heptylbiphenyl-4-ylmethyl)piperidine-4,4-dicarboxylic acid

13C NMR: (CDCl3+TFA; 100.61 MHz; ppm)

14.72 (q); 23.33 (t); 28.27 (2t); 29.84 (t); 30.03 (t); 32.05 (t); 32.49 (t); 36.37 (t); 50.52 (2t); 51.68 (s); 61.26 (t); 119.06 [d (d), JC—F=24.15 Hz]; 128.34 (d); 128.42 (s); 129.40 (4d); 132.11 (s); 132.35 (d); 132.48 (br s); 144.31 (s); 160.43 [s (d), JC—F=250.82 Hz]; 173.22 (s); 173.92 (s)

### EXAMPLE 123

### 1-(4-Decylbenzyl)piperidine-4,4-dicarboxylic acid ethyl ester

13C NMR: (DMSO-d6); 100.61 MHz; ppm)

13.82 (q); 13.95 (q); 22.11 (t); 27.17 (2t); 28.71 (2t); 28.87 (t); 29.01 (2t); 30.82 (t); 31.31 (t); 34.90 (t); 47.94 (t); 50.84 (s); 58.10 (2t); 61.45 (t); 126.95 (s); 128.51 (2d); 131.49 (2d); 143.64 (s); 169.69 (s); 170.83 (s).

### EXAMPLE 124

### 1-(3-Chloro-4-decyloxybenzyl)piperidine-4,4-dicarboxylic acid ethyl ester

13C NMR: (CDCl3+DMSO-d6); 100.61 MHz; ppm)

14.20 (q); 14.36 (q); 22.85 (t); 26.10 (t); 27.82 (2t); 29.14 (t); 29.47 (2t); 29.70 (t); 29.71 (t); 32.06 (t); 49.14 (t); 51.36 (s); 59.50 (2t); 62.22 (t); 69.42 (t); 113.68 (d); 121.26 (s); 123.16 (s); 131.68 (d); 133.21 (d); 156.00 (s); 170.05 (s); 171.64 (s)

### EXAMPLE 125

### 1-(4-Decyl-2,3-difluorobenzyl)piperidine-4,4-dicarboxylic acid ethyl ester

13C NMR: (DMSOd6+CDCl₃); 100.61 MHz; ppm)

13.87 (q); 14.05 (q); 22.51 (t); 27.41 (2t); 28.72 (t); 29.09 (t); 29.13 (t); 29.17 (t); 29.33 (t); 29.41 (t); 29.64 (t); 31.71 (t); 48.78 (t); 50.69 (s); 52.06 (2t); 61.91 (t); 114.84 (s (d), JC—F=11.01 Hz); 125.80 (d); 128.04 (d); 134.57 [s (d), JC—F=12.77 Hz]; 148.47 [s (dd), J1 C—F=247.24 Hz, J2 C—F=12.50 Hz]; 149.59 [s (dd), J1 C—F=250.06 Hz, J2 C—F=14.13 Hz]; 169.49 (s); 170.88 (s)

### EXAMPLE 126

### 1-(2-Fluoro-4′-heptylbiphenyl-4-ylmethyl)piperidine-4,4-dicarboxylic acid ethyl ester

13C NMR: (CDCl3+DMSO-d6); 100.61 MHz; ppm)

14.06 (q); 14.21 (q); 22.69 (t); 27.62 (2t); 29.19 (t); 29.33 (t); 31.45 (t); 31.83 (t); 35.71 (t); 49.22 (t); 51.08 (s); 59.39 (2t); 62.11 (t); 119.35 [d (d), J C—F=24.22 Hz); 127.72 [d (d), JC—F=3.02 Hz]; 128.73 (2d); 128.84 [2d (d), JC—F=2.77 Hz); 129.39 [s (d), JC—F=7.96 Hz]; 130.69 [s (d), JC—F=13.30 Hz]; 131.42 [d (d), JC—F=3.63 Hz]; 132.01 (s); 143.21 (s); 159.53 [s (d), JC—F=249.54 Hz]; 169.75 (s); 171.19 (s)

### EXAMPLE 127

### 1-(4-Decyl-2,3-difluorobenzyl)piperidine-4,4-dicarboxylic acid methyl ester

13C NMR: (CDCl3+DMSOd6); 100.61 MHz; ppm)

14.38 (q); 22.78 (t); 27.75 (t); 28.97 (2t); 29.35 (t); 29.40 (t); 29.44 (t); 29.61 (t); 29.68 (t); 29.92 (t); 31.98 (t); 49.07 (t); 49.67 (s); 52.26 (2t); 53.26 (q); 115.51 [s, (d, J=11.08 Hz)]; 125.98 (d); 128.32 [d, J=2.42 Hz); 134.63 [s, (d, J=12.79 Hz,)]; 148.73 [s, (dd, J1=246.94 Hz, J2=12.51 Hz)]; 149.87 [s, (dd, J1=250.16 Hz, J2=13.89 Hz)]; 170.38 (s); 171.07 (s).

### EXAMPLE 128

### 1-(4-Decyl-2,3-difluorobenzyl)piperidine-4,4-dicarboxylic acid isopropyl ester

13C NMR: (CDCl3+DMSO-d6); 100.61 MHz; ppm)

13.81 (q); 21.09 (2q); 22.23 (t); 27.15 (t); 28.42 (2t); 28.80 (t); 28.85 (t); 28.89 (t); 29.05 (t); 29.13 (t); 29.38 (t); 31.42 (t); 48.43 (s); 50.48 (t); 51.72 (2t); 69.20 (d); 114.79 [s, (d, J=11.11 Hz)]; 125.46 (d); 127.76 (d, J=2.98 Hz); 134.18[s, (d, J=12.76 Hz)]; 148.20 [s, (dd, J1=247.14 Hz, J2=12.42 Hz)]; 149.32 [s, (dd, J1=249.97 Hz, J2=14.07 Hz)]; 168.68 (s); 170.72 (s).

### EXAMPLE 129

### 1-(4-Decylbenzyl)-4-ethylcarbamoylpiperidine-4-carboxylic acid

13C NMR: (CDCl3+TFA); 100.61 MHz; ppm)

13.78 (q); 14.65 (q); 23.34 (t); 28.75 (2t); 30.00 (2t); 30.11 (t); 30.23 (t); 30.29 (t); 31.84 (t); 32.58 (t); 36.34 (t); 36.76 (t); 50.95 (t); 52.56 (s); 62.41 (2t); 124.70 (s); 130.34 (2d); 131.42 (2d); 147.23 (s); 170.79 (s); 174.73 (s)

Extra set of peaks

14.03 (q); 28.87 (2t); 31.86 (t); 36.67 (t); 49.99 (t); 50.58 (s); 61.87 (2t); 124.79 (s); 131.48 (2d); 147.17 (s); 170.14 (s); 175.49 (s)

### EXAMPLE 130

### 1-(4-Decylbenzyl)-4-propylcarbamoylpiperidine-4-carboxylic acid

13C NMR: (CDCl3+TFA); 100.61 MHz; ppm)

11.16 (q); 14.43 (q); 22.11 (t); 23.08 (t); 28.64 (2t); 29.74 (2t); 29.85 (t); 29.97 (t); 30.02 (t); 31.59 (t); 32.31 (t); 36.08 (t); 43.09 (t); 50.63 (t); 52.33 (s); 62.08 (2t); 124.58 (s); 130.06 (2d); 131.24 (2d); 146.88 (s); 170.76 (s); 175.30 (s)

Extra set of peaks

10.96 (q); 22.36 (t); 28.49 (2t); 31.57 (t); 42.97 (t); 49.67 (t); 50.29 (s); 61.53 (2t); 124.49 (s); 130.03 (2d); 131.15 (2d); 146.81 (s); 170.03 (s); 174.55 (s)

### EXAMPLE 131

### 1-(2,3-difluoro-4-nonylbenzyl)-4-ethylcarbamoyl piperidine-4-carboxylic acid

13C NMR: (CDCl3+DMSOd6); 100.61 MHz; ppm)

13.84 (q); 14.63 (q); 23.32 (t); 28.85 (2t); 29.54 (t); 29.95 (2t); 30.12 (t); 30.33 (t); 32.53 (t); 36.77 (t); 50.44 (2t); 50.99 (t); 52.21 (s); 55.14 (t); 114.51 [s, (t, J=10.34 Hz)]; 126.99 (d, J=17.03 Hz); 127.03 (d, J=17.16 Hz); 137.30 [s, (d, J=12.92 Hz, 150.79 [s, (dd, J1=249.39 Hz, J2=11.81 Hz)]; 151.64 [s, (dd, J1=250.83 Hz, J2=14.45 Hz)]; 170.13 (s); 174.22 (s) (Major Isomer)

### EXAMPLE 132

### 4-[(4-Decyl-2,3-difluorobenzyl)methylamino]cyclohexane-1,1-dicarboxylic acid diethyl ester

13C NMR: (CDCl3; 50.327 MHz; ppm)

14.69 (q); 14.78 (2q); 23.35 (t); 25.57 (br 2t); 29.39 (br 2t); 29.93 (t); 29.99 (t); 30.07 (t); 30.22 (t); 30.27 (t); 30.74 (t); 31.30 (t); 32.56 (t); 38.42 (q); 50.93 (t); 55.23 (s); 61.81 (t); 61.98 (t); 62.01 (d); 124.97 [d (t), J C—F=4.21 Hz]; 125.34 [d (t), J C—F=3.95 Hz]; 126.76 [s (d), J C—F=11.20 Hz]; 130.94 [s (d), J C—F=13.03 Hz]; 149.54 [s (dd), J1 C—F=250.65 Hz, J2 C—F=18.13 Hz]; 149.91 [s (dd), J1 C—F=249.14 Hz, J2 C—F=15.76 Hz]; 171.64 (s); 173.04 (s)

### EXAMPLE 133

### 4-[(2,3-Difluoro-4-nonylbenzyl)methylamino]cyclohexane-1,1-dicarboxylic acid diethyl ester

13C NMR: (CDCl3; 50.327 MHz; ppm)

14.69 (q); 14.78 (2q); 23.34 (t); 25.54 (br 2t); 29.39 (br 2t); 29.93 (t); 29.97 (t); 30.07 (t); 30.18 (t); 30.73 (t); 31.29 (t); 32.54 (t); 38.41 (q); 50.91 (t); 55.21 (s); 61.82 (t); 61.96 (t); 62.02 (d); 124.98 [d (t), J C—F=4.31 Hz]; 125.37 [d (t), J C—F=3.99 Hz]; 126.68 [s (d), J C—F=11.19 Hz]; 130.96 [s (d), J C—F=13.05 Hz]; 149.53 [s (dd), J1 C—F=251.01 Hz, J2 C—F=18.5 Hz]; 149.91 [s (dd), J1 C—F=249.20 Hz, J2 C—F=15.82 Hz]; 171.64 (s); 173.04 (s)

### EXAMPLE 134

### 4-(2,3-Difluoro-4-nonylbenzylamino)cyclohexane-1,1-dicarboxylic acid

13C NMR: (CDCl3+TFA; 50.327 MHz; ppm)

14.69 (q); 23.31 (t); 25.89 (2t); 29.43 (2t); 29.95 (2t); 29.99 (t); 30.02 (t); 30.17 (t); 30.41 (t); 32.53 (t); 42.10 (t); 54.42 (s); 56.44 (d); 117.29 [s (d), JC—F=11.53 Hz]; 126.33 (2d); 135.32 [s (d), JC—F=12.71 Hz]; 149.39 [s (dd), J1 C—F=245.35 Hz, J2 C—F=9.44 Hz]; 149.95 [s (dd), J1 C—F=248.55 Hz, J2 C—F=13.06 Hz]; 174.44 (s); 175.27 (s)

### EXAMPLE 135

### 4-[(2,3-Difluoro-4-nonylbenzyl)methylamino]cyclohexane-1,1-dicarboxylic acid ethyl ester

13C NMR: (DMSO-d6; 50.327 MHz; ppm)

13.93 (2q); 22.08 (t); 22.85 (br 2t); 28.00 (br 2t); 28.59 (t); 28.66 (t); 28.71 (t); 28.88 (t); 29.31 (2t); 31.25 (t); 35.85 (q); 48.02 (t); 53.17 (s); 60.97 (t); 62.17 (d); 117.20 [s (d), JC—F=10.5 Hz]; 125.41 (d); 128.08 (d); 133.09 [s (d), JC—F=12.60 Hz]; 148.01 [s (dd), J1 C—F=244.55 Hz, J2 C—F=12.38 Hz]; 149.10 [s (dd), J1 C—F=249.15 Hz, J2 C—F=13.68 Hz]; 170.03 (s); 172.36 (s)

### EXAMPLE 136

### 4-[(4-Decyl-2,3-difluorobenzyl)methylamino]cyclohexane-1,1-dicarboxylic acid ethyl ester (264)

13C NMR: (DMSO-d6; 50.327 MHz; ppm)

13.91 (2q); 22.07 (t); 22.85 (br 2t); 27.98 (br 2t); 28.57 (t); 28.66 (2t); 28.91 (t); 28.93 (t); 29.29 (2t); 31.26 (t); 35.85 (q); 48.05 (t); 53.16 (s); 60.95 (t); 62.16 (d); 117.24 [s (d), JC—F=10.49 Hz]; 125.39 (d); 128.03 (d); 133.05 [s (d), JC—F=12.78 Hz]; 147.99 [s (dd), J1 C—F=244.21 Hz, J2 C—F=12.42 Hz]; 147.99 [s (dd), J1 C—F=249.23 Hz, J2 C—F=13.63 Hz]; 170.01 (s); 172.34 (s)

### EXAMPLE 137

### 4-(4-Decyl-2,3-difluorobenzylamino)cyclohexane-1,1-dicarboxylic acid

13C NMR: (TFA+CDCl3; 100.61 MHz; ppm) 14.72 (q); 23.34 (t); 25.90 (br 2t); 29.45 (br 2t); 29.95 (t); 29.98 (2t); 30.19 (t); 30.25 (t); 30.37 (2t); 32.56 (t); 42.72 (t); 54.25 (s); 56.80 (d); 116.84 [s (d), JC—F=8.70 Hz]; 126.14 (d); 126.57 (d); 135.91 [s (d), JC—F=11.76 Hz]; 149.49 [s (dd), J1 C—F=250.51 Hz, J2 C—F=13.52 Hz]; 149.96 [s (dd), J1 C—F=252.02 Hz, J2 C—F=16.54 Hz]; 175.02 (s); 176.17 (s)

### EXAMPLE 138

### 4-[(2,3-Difluoro-4-nonylbenzyl)methylamino]cyclohexane-1,1-dicarboxylic acid

13C NMR: (CDCl3+TFA; 50.327 MHz; ppm)

12.64 (q); 20.85 (t); 23.09 (2t); 26.75 (2t); 27.35 (t); 27.42 (t); 27.50 (t); 27.65 (t); 28.25 (t); 29.37 (t); 30.02 (t); 35.69 (q); 48.51 (t); 51.61 (s); 59.96 (d); 123.28 (d); 123.97 (d); 128.59 [s (d), JC—F=12.74 Hz]; 146.90 [s (dd), J1 C—F=243.81 Hz, J2 C—F=12.42 Hz]; 147.25 [s (dd), J1 C—F=246.13 Hz, J2 C—F=13.47 Hz]; 172.42 (s); 173.46 (s) One singlet is merged with δ 123.97

### EXAMPLE 139

### 4-[(4-Decyl-2,3-difluorobenzyl)methylamino]cyclohexane-1,1-dicarboxylic acid

13C NMR: (DMSO; 100.61 MHz; ppm)

13.90 (q); 22.07 (t); 24.42 (br, 2t); 27.88 (br, 2t); 28.53 (t); 28.65 (t); 28.90 (t); 28.92 (t); 29.44 (t); 30.39 (t); 30.67 (t); 31.26 (t); 36.97 (q); 49.88 (t); 52.80 (s); 61.07 (d); 124.68 (d); 125.28 (d); 125.73 [s (d), JC—F=10.29 Hz]; 129.72 [s (d), JC—F=12.69 Hz]; 148.01 [s (dd), J1 C—F=243.42 Hz, J2 C—F=12.37 Hz]; 148.42 [s (dd), J1 C—F=245.85 Hz, J2 C—F=13.78 Hz]; 173.67 (s); 174.70 (s)

### EXAMPLE 140

### 1-[4-(4-Phenyl-5-trifluoromethylthiophen-2-ylmethoxy)benzyl]-piperidine-4,4-dicarboxylic acid

13C NMR: (TFA+CDCl3; 100.61 MHz; ppm)

28.33 (2t); 50.43 (2t); 51.74 (s); 62.38 (t); 65.58 (t); 116.77 (2d); 120.41 (s); 123.18 [s (q), JC—F=269.92 Hz]; 126.56 [s (q), JC—F=36.76 Hz]; 129.09 (2d); 129.15 (d); 129.39 (2d); 130.97 (d); 133.28 (2d); 134.97 (s); 140.89 (s); 144.96 [s (q), JC—F=2.75 Hz]; 160.68 (s); 174.45 (s); 174.70 (s)

### EXAMPLE 141

### 1-(4-Decylbenzyl)azetidine-3,3-dicarboxylic acid

13C NMR: (TFA+CDCl3; 100.61 MHz; ppm)

14.68 (q); 23.35 (t); 30.01 (2t); 30.11 (t); 30.25 (t); 30.30 (t); 31.85 (t); 32.59 (t); 36.34 (t); 48.09 (s); 58.75 (2t); 60.33 (t); 124.92 (s); 130.48 (2d); 130.58 (2d); 147.29 (s); 171.07 (2s)

### EXAMPLE 142

### 2-[4-(3-Chloro-4-decyloxybenzylamino)but-2-ynyl]-2-methyl malonic acid

13C NMR: (CDCl3+TFA); 100.61 MHz; δ ppm)

14.71 (q); 20.67 (q); 23.35 (t); 26.53 (t); 26.61 (t); 29.59 (t); 29.97 (t); 29.99 (t); 30.21 (2t); 32.58 (t); 36.97 (t); 50.49 (t); 53.93 (s); 70.16 (t); 72.36 (s); 86.41 (s); 114.36 (d); 121.57 (s); 124.61 (s); 130.31 (d); 132.40 (d); 157.00 (s); 176.25 (2s).

### EXAMPLE 143

### 2-{4-[(2-Fluoro-4′-heptylbiphenyl-4-ylmethyl)amino]but-2-ynyl}-2-methylmalonic acid

13C NMR: (TFA+CDCl3; 100.61 MHz; ppm)

14.69 (q); 20.64 (q); 23.36 (t); 26.60 (t); 29.88 (t); 30.04 (t); 32.08 (t); 32.52 (t); 36.41 (t); 37.41 (t); 50.70 (t); 54.02 (s); 72.28 (s); 86.59 (s); 118.35 (d (d), JC—F=23.34 Hz); 126.50 (d); 129.43 (4d); 132.22 (s); 132.37 (s (d), JC—F=13.29 Hz); 132.68 (d (d), JC—F=3.30 Hz); 144.31 (s); 160.56 (s (d) JC—F=251.00 Hz); 176.50 (2s) One singlet is merged with the doublet at ˜130

### EXAMPLE 144

### 2-{4-[Isopropyl-(4-nonylbenzyl)amino]but-2-ynyl}-2-methylmalonic acid

13C NMR: (TFA+CDCl3; 100.61 MHz; ppm)

14.68 (q); 18.05 (q); 18.15 (q); 20.96 (q); 23.32 (t); 26.87 (t); 29.98 (t); 30.10 (t); 30.17 (t); 31.81 (t); 32.55 (t); 36.33 (t); 40.19 (t); 54.01 (s); 55.12 (t); 57.63 (d); 71.46 (s); 87.95 (s); 125.49 (s); 130.42 (2d); 131.00 (2d); 146.96 (s); 176.50 (2s)

Probably one triplet is merged at ˜30.

### EXAMPLE 145

### 2-{4-[Cyclopropyl-(4-nonylbenzyl)amino)but-2-ynyl}-2-methylmalonic acid

13C NMR: (TFA+CDCl3; 100.61 MHz; ppm)

5.42 (t); 6.62 (t); 14.67 (q); 21.21 (q); 23.26 (t); 27.23 (t); 29.91 (t); 29.96 (t); 30.06 (t); 30.13 (t); 31.84 (t); 32.48 (t); 36.33 (t); 38.35 (d); 45.12 (t); 53.79 (s); 58.87 (t); 71.47 (s); 88.09 (s); 124.87 (s); 130.03 (2d); 132.21 (2d); 146.52 (s); 175.41 (2s)

### EXAMPLE 146

### 2-{4-[(2-Fluoro-4′-hexylbiphenyl-4-ylmethyl)methylamino]but-2-ynyl}-2-methylmalonic acid

13C NMR: (DMSO-d6; 100.61 MHz; ppm)

13.94 (q); 20.36 (q); 22.07 (t); 26.01 (t); 28.39 (t); 30.86 (t); 31.12 (t); 34.85 (t); 40.86 (q); 44.66 (t); 52.25 (s); 57.86 (t); 75.99 (s); 82.56 (s); 116.41 (d (d), JC—F=23.04 Hz); 125.46 (d); 127.10 (s (d), JC—F=13.10 Hz); 128.51 (2d); 128.56 (2d); 130.42 (d); 132.24 (s); 139.24 (s); 142.06 (s); 158.95 (s (d), JC—F=245.84 Hz); 172.93 (2s)

### EXAMPLE 147

### 2-Methyl-2-{4-[methyl-(4-nonylbenzyl)amino]but-2-ynyl}malonic acid

13C NMR: (CDCl3+TFA); 100.61 MHz; δ ppm)

14.74 (q); 21.06 (q); 23.33 (t); 26.96 (t); 29.98 (t); 30.02 (t); 30.12 (t); 30.19 (t); 31.88 (t); 32.54 (t); 36.35 (t); 40.21 (q); 44.74 (t); 53.79 (s); 58.87 (t); 70.69 (s); 88.71 (s); 125.92 (s); 130.17 (2d); 131.30 (2d); 146.45 (s); 175.02 (s); 175.07 (s).

### EXAMPLE 148

### 2-{4-[(2,3-Difluoro-4-nonyl benzyl)methylamino]but-2-ynyl}-2-methyl malonic acid

13C NMR: (CDCl3+TFA); 100.61 MHz; δ ppm)

14.51 (q); 20.67 (q); 23.40 (t); 26.68 (t); 29.66 (t); 30.07 (3t); 30.23 (t); 30.45 (t); 32.65 (t); 40.80 (q); 46.69 (t); 53.15 (t); 54.21 (s); 70.72 (s); 88.61 (s); 114.53 [s, (d, J=11.28 Hz)]; 126.93 (d, J=3.27 Hz); 127.29 (d, J=3.95 Hz); 137.80 [s, (d, J=12.89 Hz)]; 149.95 [s, (dd), J1=247.47 Hz, J2=9.47 Hz)]; 150.64 [s, (dd), J1=258.90 Hz J2=22.21 Hz]; 177.26 (2s).

### EXAMPLE 149

### 2-{4-[Ethyl-(4-nonylbenzyl)amino]but-2-ynyl}-2-methyl malonic acid

13C NMR: (CDCl3+TFA); 100.61 MHz; δ ppm)

10.00 (q); 14.71 (q); 20.87 (q); 23.33 (t); 26.80 (t); 29.98 (2t); 30.11 (t); 30.17 (t); 31.83 (t); 32.55 (t); 36.34 (t); 41.07 (t); 49.22 (t); 53.92 (s); 57.77 (t); 70.81 (s); 87.79 (s); 125.07 (s); 130.39 (2d); 131.16 (2d); 147.06 (s); 176.47 (2s).

### EXAMPLE 150

### 2-{4-[(2,3-Difluoro-4-nonyl benzyl)amino]-but-2-ynyl}-2-methyl malonic acid

13C NMR: (CDCl3+TFA); 100.61 MHz; δ ppm)

14.58 (q); 20.41 (q); 23.38 (t); 26.48 (t); 29.59 (t); 30.00 (t); 30.04 (2t); 30.18 (t); 30.42 (t); 32.62 (t); 38.15 (t); 45.21 (t); 54.08 (s); 72.19 (s); 86.47 (s); 115.68 [s, (d, J=11.55 Hz); 126.97 (d); 127.00 (d); 137.11 [s, (d, J=12.90 Hz)]; 149.76 [s, (dd, J1=254.97 Hz, J2=17.30 Hz)]; 150.33 [s, (dd, J1=246.81 Hz, J2=11.17 Hz)]; 177.18 (2s).

### EXAMPLE 151

### 2-[4-(4-Decyl-2,3-difluorobenzylamino)but-2-ynyl]-2-methylmalonic acid

13C NMR: (TFA+CDCl3; 100.61 MHz; ppm)

14.67 (q); 20.51 (q); 23.38 (t); 26.51 (t); 29.56 (t); 29.96 (t); 30.02 (2t); 30.20 (t); 30.29 (t); 30.38 (t); 32.60 (t); 38.10 (t); 45.11 (t); 54.01 (s); 72.20 (s); 86.41 (s); 115.68 [s (d), JC—F=11.42 Hz]; 126.25 (d); 126.89 (d); 136.92 [s (d); J=12.79 Hz]; 149.68 [s (dd), J1 C—F=251.739 Hz, J2 C—F=13.95 Hz]; 150.26 [s (dd), J1 C—F=256.293 Hz, J2 C—F=20.94 Hz]; 176.93 (2s)

### EXAMPLE 152

### 2-{4-[(4-Decylbenzyl)methylamino]but-2-ynyl}-2-methylmalonic acid

13C NMR: (TFA+CDCl3; 100.61 MHz; ppm)

14.71 (q); 20.85 (q); 23.34 (t); 26.79 (t); 29.99 (t); 30.00 (t); 30.10 (t); 30.22 (t); 30.27 (t); 31.84 (t); 32.56 (t); 36.34 (t); 40.31 (q); 45.20 (t); 53.86 (s); 59.44 (t) 72.84 (s); 88.47 (s); 125.13 (s); 130.36 (2d); 131.26 (2d); 147.03 (s); 175.78 (2s)

### EXAMPLE 153

### 2-{4-[(3-Chloro-4-nonyloxy benzyl)methylamino]but-2-ynyl}-2-methyl malonic acid

13C NMR: (CDCl3+TFA); 100.61 MHz; δ ppm)

14.69 (q); 20.91 (q); 23.33 (t); 26.52 (t); 26.80 (t); 29.56 (t); 29.90 (t); 29.95 (t); 30.15 (t); 32.53 (t); 40.41 (q); 45.22 (t); 53.87 (s); 58.66 (t); 70.16 (t); 70.79 (s); 80.30 (s); 114.40 (d); 120.43 (s); 124.64 (s); 131.07 (d); 133.04 (d); 157.30 (s); 176.05 (2s).

### EXAMPLE 154

### 2-Ethyl-2-{4-[(2-fluoro-4′-hexylbiphenyl-4-ylmethyl)amino]but-2-ynyl}malonic acid

13C NMR: (TFA+CDCl3; 100.61 MHz; ppm)

9.18 (q); 14.72 (q); 23.28 (t); 24.45 (t); 28.23 (t); 29.73 (t); 32.02 (t); 32.39 (t); 36.38 (t); 37.06 (t); 50.32 (t); 58.44 (s); 72.29 (s); 86.47 (s); 118.34 (d (d), JC—F=24.36 Hz); 126.56 (d); 129.37 (4d); 129.85 (s (d), JC—F=7.56 Hz); 132.01 (s (d), JC—F=13.20 Hz); 132.27 (d); 132.52 (s); 144.16 (s); 160.46 (s (d), JC—F=250.78 Hz); 175.77 (2s)

### EXAMPLE 155

### 2-Methyl-2-{4-[4-(4-phenyl-5-trifluoromethylthiophen-2-ylmethoxy)benzyamino]but-2-ynyl}malonic acid

13C NMR: (TFA+CDCl3; 100.61 MHz; ppm)

20.50 (q); 26.54 (t); 37.48 (t); 51.64 (t); 54.11 (s); 65.70 (t); 72.29 (s); 86.48 (s); 116.93 (2d); 121.83 (s); 123.25 [s (q), JC—F=269.755 Hz]; 126.75 [s (q), JC—F=36.80 Hz]; 129.14 (2d); 129.20 (d); 129.44 (d); 129.45 (d); 131.06 (d); 132.37 (2d); 135.08 (s); 140.92 (s); 145.06 [s (q), JC—F=2.91 Hz]; 160.55 (s); 177.06 (2s)

Biological Testing

Human S1P1-5 receptor subtypes were stably expressed in HEK 293 or CHOK1 cells following transfection with corresponding plasmid constructs. Although the native cells somewhat respond to S1P, the level of expression and responsiveness of the antibiotic-resistant transfected cell lines that were selected is much higher.

When cultivated cells reached 80% confluence, they were collected and, after lysis, cell membranes were collected by centrifugation and washed in buffer containing antiproteases.

Binding assays were performed using 5-20 μg of cell membranes suspended in 20 mM tris-HCl pH 7.4 containing 15 mM NaF and 2.5 mM deoxypyridoxine in a final volume of 250 μl. The radioligand was 0.5 nM 3H-D-erythro-dihydro-S1P incubated in the presence of bovine serum albumin for 1 h after or 2 h-preincubation. Non specific binding was defined from incubations in the presence of 5 μM S1P.

GTP-γ-35 S binding was performed using ˜5 μg protein of cell membranes suspended in 50 mM tris-HCl pH 7.5 containing 10 mM Mg Cl2, 100 mM NaCl and 10 μM GDP. The radioligand was 0.025 nM [35S] GTP-γ-S and non specific binding determined in the presence of 10 μM non-radioactive GTP-γ-S. S1P and receptor agonists enhance the specific binding whereas inverse agonists reduce it. The maximal stimulation elicited by S1P was taken as a reference to define full or partial agonism and calculate the intrinsic activity (i.a.) of compounds.

Typical results shown in Table 1 indicate that compounds of the invention are able to activate S1P1 (and sometimes S1P2) receptors with a potency similar to that of S1P itself (i.e. with full intrinsic activity and at nanomolar concentrations) without affecting significantly S1P3 receptor.

TABLE 1
Agonist activity of compounds and on S1P1, S1P3 and S1P2 receptors

| | EC50 (nM) | EC50 (nM) | EC50 (nM) |
|---|---|---|---|
| | S1P1 | S1P3 | S1P2 |
| 3 | 6 (i.a = 1) | >1000 (i.a~0.3) | 4 (i.a = 1) |
| 12 | 13 (i.a = 1) | >1000 (i.a~0.3) | 5 (i.a = 1) |

1. A compound of formula (I): 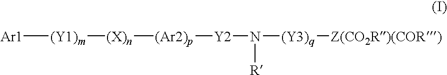
wherein
Ar1 represents an Aryl or Heteroaryl group optionally substituted by 1 up to 5 R group(s),
wherein, each R, identical or different, is selected from the group consisting in Halogen atom, perhalogenoalkyl, -Alkyl, —OAlkyl, —OH, —COOR7, —CONR7R8, -Alkyl-Hal, —OAlkyl-Hal, NO2, —CN, —NR7R8, -AlkylAryl, -Aryl, —S(O)lR7, -Alkenyl, —Si(Alkyl)₃;
wherein R7 and R8, identical or different, are chosen from the group consisting in H Alkyl; Cycloalkyl; Aryl; -AlkylAryl; Heteroaryl; wherein Alkyl, Cycloalkyl, and/or Aryl is(are) optionally substituted by one or more identical or different Halogen atom, polyfluoroalkyl, Aryl, —COOR7
or R7 and R8 form together with the N atom to which they are attached a Heterocycle;
l is 0, 1 or 2;
Y1 represents an -Alkyl-chain, optionally substituted by one or more R as defined above, and optionally comprising one or more unsaturation(s) and/or heteroatom(s) and/or a residue chosen from the group consisting in -Alkenyl-, -Alkynyl-, —CH═N—, —N═CH—, —CH═N—O—, —O—N═CH—, —C(═O)—, —C(═O)(OR7)-, —C(═O)(NR7)-, —N═N—, —S(O)l-;
m is 0 or 1;
X represents a heteroatom;
n is 0 or 1;
—Ar2- represents an -Aryl-group optionally substituted by one or more R group as defined above or wherein 2 R may form together with the atoms to which they are attached a fused cyclic, aryl or heteroaryl ring;
p is 0 or 1;
—Y2- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7), —N═N—, —S(O)l-;
R′represents a hydrogen atom, or a -Cycloalkyl or an -Alkyl chain, the cycloalkyl or alkyl being optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)O—, —C(═O)(NR7)-, —N═N—, —S(O)l-, such as -Alkenyl-Alkyl, -Alkynyl-Alkyl, —CH═N-Alkyl, —N═CH-Alkyl, —CH═N—O-Alkyl, —O—N═CH-Alkyl, —C(═O)-Alkyl, —C(═O)O-Alkyl, —C(═O)(NR7)-Alkyl, —N═N-Alkyl, —S(O)l-Alkyl;
—Y3- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7)-, —N═N—, —S(O)l-;
q is 0 or 1;
Z represents a -Cycloalkyl< group or an -Aryl<, -Heteroaryl< group or a —C(alkyl)< group, or R′ and Z form together with the N atom to which they are attached a Heterocyclic or Heteroaryl group;
wherein (CO2R″) and (COR′″) can be attached to the same atom or two adjacent atoms;
—R″ represents a hydrogen atom or an Alkyl chain, optionally substituted by one or more R as defined above, and optionally comprising one or more unsaturation(s);
—R′″ represents —OH, —OAlkyl, H, —NR7OR8, —NR7R8, natural or synthetic amino acids, wherein R7 and R8 are defined as above;
or R″ and R′″ form together a ring with the atom(s) to which they are attached to form a cyclic intramolecular bis carbonyl group, including Meldrum acid derivatives;
as well as their enantiomers, diatereoisomers, geometrical isomers, mixtures thereof, free forms and pharmaceutically acceptable salts, hydrates, solvates and esters
with the exception of compounds where:
m=n=p=0, q=0 or 1, R′═H or Alkyl, Y2=-Alkyl-, Y3=-Alkyl-, Z=−C(Alkyl)< and Ar1=unsubstituted phenyl or phenyl substituted with one or more identical R where R is Halogen; and
m=n=p=0, q=1, R′═H, Y2=—CH₂—, Y3=—CH₂—, Z=-cyclobutyl< and Ar1=unsubstituted phenyl.

2. Compound according to claim 1 represented by the following general formula (II): 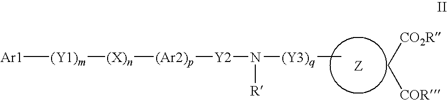
in which Ar1, Y1, X, Ar2, Y2, R, R′, Y3, R″, R′″, m, n, p, q are defined as in formula (I) and 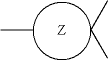
represents a -Cycloalkyl< group or an -Aryl<, -Heteroaryl< group;
wherein (CO2R″) and (COR′″) can be attached to the same atom or two adjacent atoms of the Z ring;
as well as their enantiomers, diatereoisomers, geometrical isomers, mixtures thereof, free forms and pharmaceutically acceptable salts, hydrates, solvates and esters.

3. Compound according to claim 1, wherein:
Ar1 represents an Aryl or Heteroaryl group optionally substituted by 1 up to 5 R group(s),
wherein each R, identical or different, is selected from the group consisting in Halogen atom, perhalogenoalkyl, -Alkyl, —OAlkyl;
Y1 represents an -Alkyl-chain or a —S-Alkyl-chain;
m is 0 or 1;
X represents a heteroatom;
n is 0 or 1;
—Ar2- represents an -Aryl-group;
p is 0 or 1;
—Y2- represents an -Alkyl-chain;
—R′ represents a hydrogen atom or a cycloalkyl or an alkyl chain;
q is 0 or 1;
Y3 represents an alkynyl chain;
Z represents a —C(alkyl)< group or 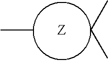
represents a -Cycloalkyl< group;
—R″ represents a hydrogen atom; —R′″ represents —OH, —OAlkyl, —NR7R8;
(CO2R″) and (COR′″) are attached to the same atom of the Z ring;
as well as their enantiomers, diatereoisomers, geometrical isomers, mixtures thereof, free forms and pharmaceutically acceptable salts, hydrates, solvates and esters.

4. Compound according to claim 1, wherein Ar1 represents a Phenyl or Thienyl group optionally substituted by 1 up to 5 R group(s),
wherein each R, identical or different, is selected from the group consisting in Halogen atom, perhalogenoalkyl Alkyl, —OAlkyl.

5. Compound according to claim 1, wherein Y1 represents an -Alkyl-chain or a —S-Alkyl-chain.

6. Compound according to claim 1, wherein m is 0.

7. Compound according to claim 1, wherein X represents an Oxygen atom.

8. Compound according to claim 1, wherein n is 0.

9. Compound according to claim 1, wherein —Ar2- represents an -Phenyl-group.

10. Compound according to claim 1, wherein p is 0.

11. Compound according to claim 1, wherein —Y2- represents an -Alkyl-chain.

12. Compound according to claims claim 1, wherein —R′ represents a hydrogen atom or a cycloalkyl or an alkyl chain, or —COOAlkyl.

13. Compound according to claim 1, wherein q is 0.

14. Compound according to 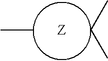
claim 2, wherein represents a -Cycloalkyl< group wherein (CO2R″) and (COR′″) are attached to the same atom of the Z ring.

15. Compound according to claim 1, wherein —R″ represents a hydrogen atom.

16. Compound according to claim 1, wherein —R′″ represents —OH, —OAlkyl, —NR7R8.

17. Compound according to claim 1, selected from the group consisting in:
3-(4-octylbenzylamino)cyclopentane-1,1-dicarboxylic acid
3-(4-octylbenzylamino)cyclopentane-1,1-dicarboxylic acid ethyl ester
3-(4-nonylbenzylamino)cyclopentane-1,1-dicarboxylic acid
3-(4-octyloxybenzylamino)cyclopentane-1,1-dicarboxylic acid
3-(3-methoxy-4-octyloxybenzylamino)cyclopentane-1,1-dicarboxylic acid
3-(3-methoxy-4-butyloxybenzylamino)cyclopentane-1,1-dicarboxylic acid
3-(3-methoxy-4-hexylyloxybenzylamino)cyclopentane-1,1-dicarboxylic acid
3-(3-bromo-4-octyloxy-5-methoxybenzylamino)cyclopentane-1,1-dicarboxylic acid
3-(3-methoxy-4-octyloxy-5-methylbenzylamino)cyclopentane-1,1-dicarboxylic acid
3-(3-chloro-4-octyloxy-5-methoxybenzylamino)cyclopentane-1,1-dicarboxylic acid
3-(4-nonylbenzylamino)cyclopentane-1,1-dicarboxylic acid
3-(4-decylbenzylamino)cyclopentane-1,1-dicarboxylic acid
3-(3-methoxy-4-nonyloxybenzylamino)cyclopentane-1,1-dicarboxylic acid
3-(3-chloro-4-nonyloxybenzylamino)cyclopentane-1,1-dicarboxylic acid
3-[4-[4-(4-methoxyphenyl)butoxy]benzylamino]cyclopentane-1,1-dicarboxylic acid
3-[4-[4-(3-chloro-4-methoxyphenyl)butoxy]benzylamino]-cyclopentane-1,1-dicarboxylic acid
3-(4-nonylbenzylamino)cyclopentane-1,1-dicarboxylic acid ethyl ester hydrochloride
3-{4-[3-fluoro-(4-hexylphenyl)benzyl]amino}cyclopentane-1,1-dicarboxylic acid hydrochloride
1-(2,2,2-trifluoroethylcarbamoyl)-3-(4-nonylbenzylamino)cyclopentanecarboxylic acid hydrochloride (isomer A)
1-(2,2,2-trifluoroethylcarbamoyl)-3-(4-nonylbenzylamino)cyclopentanecarboxylic acid hydrochloride (isomer B)
3-(4-decyloxybenzylamino)cyclopentane-1,1-dicarboxylic acid hydrochloride
1-(methylcarbamoyl)-3-(4-nonylbenzylamino)cyclopentanecarboxylic acid hydrochloride (isomer A)
1-(methylcarbamoyl)-3-(4-nonylbenzylamino)cyclopentanecarboxylic acid hydrochloride (isomer B)
3-(methyl-4-nonylbenzylamino)cyclopentane-1,1-dicarboxylic acid hydrochloride
3-[4-(4-phenylsulfanylbutoxy)benzylamino]cyclopentane-1,1-dicarboxylic acid hydrochloride
3-[4-[4-(4-ethoxy-3-chlorophenyl)butoxy]benzylamino]cyclopentane-1,1-dicarboxylic acid hydrochloride
2-methyl-2-[4-(4-nonylbenzylamino)but-2-ynyl]malonic acid hydrochloride
2-[4-(3-chloro-4-nonyloxybenzylamino)but-2-ynyl]-2-methylmalonic acid hydrochloride
2-methyl-2-[4-(4-octylbenzylamino)but-2-ynyl]malonic acid hydrochloride
2-[4-(4-decylbenzylamino)but-2-ynyl]-2-methylmalonic acid hydrochloride
2-[3-fluoro-4-(4-hexylphenyl)benzylamino]but-2-ynyl]-2-methylmalonic acid hydrochloride
3-[(2-Fluoro-4′-heptylbiphenyl-4-ylmethyl)amino]cyclopentane-1,1-dicarboxylic acid
1-Carbamoyl-3-(4-nonylbenzylamino)cyclopentanecarboxylic acid—Isomer B
3-(3-Chloro-4-decyloxybenzylamino)cyclopentane-1,1-dicarboxylic acid
3-(3-Chloro-4-octyloxybenzylamino)cyclopentane-1,1-dicarboxylic acid
1-Carbamoyl-3-(4-nonylbenzylamino)cyclopentanecarboxylic acid—Isomer A
3-[Cyclopropyl-(4-nonylbenzyl)amino]cyclopentane-1,1-dicarboxylic acid
3-[(2-Fluoro-4′-heptylbiphenyl-4-ylmethyl)methylamino]cyclopentane-1,1-dicarboxylic acid
3-(2,3-Difluoro-4-nonylbenzylamino)cyclopentane-1,1-dicarboxylic acid
3-(2,3-Difluoro-4-nonylbenzylamino)cyclopentane-1,1-dicarboxylic acid ethyl ester
3-(4-Nonyl-benzylamino)cyclopentane-1,1-dicarboxylic acid isobutyl ester
3-[Methyl-(4-nonylbenzyl)amino]cyclopentane-1,1-dicarboxylic acid ethyl ester
3-(4-Nonylbenzylamino)cyclopentane-1,1-dicarboxylic acid methyl ester
3-(4-Nonylbenzylamino)cyclopentane-1,1-dicarboxylic acid benzyl ester
3-[(2-Fluoro-4′-octylbiphenyl-4-ylmethyl)amino]cyclopentane-1,1-dicarboxylic acid ethyl ester
3-[(2-Fluoro-4′-octylbiphenyl-4-ylmethyl)amino]cyclopentane-1,1-dicarboxylic acid
3-[Cyclopropyl-(2-fluoro-4′-heptylbiphenyl-4-ylmethyl)amino]cyclopentane-1,1-dicarboxylic acid
3-(4-Nonylbenzylamino)cyclopentane-1,1-dicarboxylic acid isopropylester
3-(2,3-Difluoro-4-nonylbenzylamino)-1-methylcarbamoylcyclopentane carboxylic acid—Isomer A
3-(2,3-Difluoro-4-nonylbenzylamino)-1-methylcarbamoylcyclopentane carboxylic acid—Isomer B
3-(4-Nonylbenzylamino)-1-propylcarbamoylcyclopentanecarboxylic acid—Isomer A
3-(4-Nonylbenzylamino)-1-propylcarbamoylcyclopentanecarboxylic acid—Isomer B
3-(4-Decyl-2,3-difluorobenzylamino)cyclopentane-1,1-dicarboxylic acid
3-(4-Decylbenzylamino)-1-methylcarbamoylcyclopentanecarboxylic acid—Isomer A
3-(4-Decylbenzylamino)-1-methylcarbamoylcyclopentanecarboxylic acid—Isomer B
1-Ethylcarbamoyl-3-(4-nonylbenzylamino)cyclopentanecarboxylic acid—Isomer A
1-Ethylcarbamoyl-3-(4-nonylbenzylamino)cyclopentanecarboxylic acid—Isomer B
1-Isopropylcarbamoyl-3-(4-nonylbenzylamino)cyclopentanecarboxylic acid-Isomer A
1-Isopropylcarbamoyl-3-(4-nonylbenzylamino)cyclopentanecarboxylic acid-Isomer B
3-(4-Decyl-2,3-difluorobenzylamino)-1-methylcarbamoylcyclopentanecarboxylic acid—Isomer A
3-[(2,3-Difluoro-4-nonylbenzyl)methylamino]-1-methylcarbamoylcyclopentanecarboxylic acid—Isomer A
3-[(2,3-Difluoro-4-nonylbenzyl)methylamino]-1-methylcarbamoylcyclopentanecarboxylic acid—Isomer B
3-[(4-Decylbenzyl)methylamino]cyclopentane-1,1-dicarboxylic acid
3-(4-Nonylbenzylamino)cyclopentane-1,1-dicarboxylic acid butyl ester
3-(4-Nonylbenzylaminocyclopentane-1,1-dicarboxylic acid propyl ester
1-(3,4-Difluorophenyl carbamoyl)-3-(4-nonylbenzylamino)cyclopentanecarboxylic acid
3-[(2,3-Difluoro-4-nonylbenzyl)methylamino]cyclopentane-1,1-dicarboxylic acid
3-(4-Decyl-2,3-difluorobenzylamino)cyclopentane-1,1-dicarboxylic acid isopropyl ester
3-(4-Decylbenzylamino)cyclopentane-1,1-dicarboxylic acid isopropyl ester
3-[(2-Fluoro-4′-heptylbiphenyl-4-ylmethyl)amino]-1-methylcarbamoylcyclopentanecarboxylic acid—Isomer A
3-[(2-Fluoro-4′-heptylbiphenyl-4-ylmethyl)amino]-1-methylcarbamoylcyclopentanecarboxylic acid—Isomer B
3-(4-Decylbenzylamino)cyclopentane-1,1-dicarboxylic acid enantiomer A
3-(4-Decylbenzylamino)cyclopentane-1,1-dicarboxylic acid enantiomer B
3-[(3-Chloro-4-decyloxybenzyl)methylamino]cyclopentane-1,1-dicarboxylic acid
3-[(3-Chloro-4-nonylloxybenzyl)methylamino]cyclopentane-1,1-dicarboxylic acid
1-Dimethylcarbamoyl-3-(4-nonylbenzylamino)cyclopentanecarboxylic acid
1-Ethylcarbamoyl-3-(4-octylbenzylamino)cyclopentanecarboxylic acid—Isomer A
1-Ethylcarbamoyl-3-(4-octylbenzylamino)cyclopentanecarboxylic acid—Isomer B
1-Methylcarbamoyl-3-(4-octylbenzylamino)cyclopentanecarboxylic acid—Isomer A
1-Methylcarbamoyl-3-(4-octylbenzylamino)cyclopentanecarboxylic acid—Isomer B
3-(4-octylbenzylamino)cyclopentane-1,1-dicarboxylic acid isopropyl ester
3-[(4-Decyl-2,3-difluorobenzyl)methylamino]cyclopentane-1,1-dicarboxylic acid
3-(4-Decyl-2,3-difluorobenzylamino)cyclopentane-1,1-dicarboxylic acid enantiomer B
3-(4-Decyl-2,3-difluorobenzylamino)cyclopentane-1,1-dicarboxylic acid enantiomer A
3-[(2-Fluoro-4′-heptylbiphenyl-4-ylmethyl)amino]cyclopentane-1,1-dicarboxylic acid enantiomer B
3-(4-Decylbenzylamino)cyclopentane-1,1-dicarboxylic acid ethyl ester—Isomer A
3-(4-Decylbenzylamino)cyclopentane-1,1-dicarboxylic acid ethyl ester—Isomer B
3-[(4-Decylbenzyl)methylamino]cyclopentane-1,1-dicarboxylic acid ethyl ester
3-[(4-Decyl-2,3-difluorobenzyl)methylamino]cyclopentane-1,1-dicarboxylic acid enantiomer B
3-[(4-Decyl-2,3-difluorobenzyl)methylamino]cyclopentane-1,1-dicarboxylic acid ethyl ester enantiomer B
3-[(4-Decyl-2,3-difluorobenzyl)methylamino]cyclopentane-1,1-dicarboxylic acid enantiomer A
3-[(4-Decyl-2,3-difluorobenzyl)methylamino]cyclopentane-1,1-dicarboxylic acid ethyl ester enantiomer A
3-(4-Decyl-2,6-difluorobenzylamino)cyclopentane-1,1-dicarboxylic acid
3-[(4-Decyl-2,6-difluorobenzyl)methylamino]cyclopentane-1,1-dicarboxylic acid
3-[(4-Decyl-2,3-difluorobenzyl)ethoxycarbonylamino]cyclopentane-1,1-dicarboxylic acid
3-[(4-Decyl-2,3-difluorobenzyl)ethoxycarbonylamino]cyclopentane-1,1-dicarboxylic acid ethyl ester—Isomer A
3-[(4-Decyl-2,3-difluorobenzyl)ethoxycarbonylamino]cyclopentane-1,1-dicarboxylic acid ethyl ester—Isomer B
3-[(4-Decyl-2,3-difluorobenzyl)methoxycarbonylamino]cyclopentane-1,1-dicarboxylic acid
3-[(4-Decyl-2,3-difluorobenzyl)methoxycarbonylamino]cyclopentane-1,1-dicarboxylic acid ethyl ester
2-Methyl-2-[4-(4-nonylbenzylamino)but-2-enyl]malonic acid
3-[2-(4-Octylphenyl)ethylamino]cyclopentane-1,1-dicarboxylic acid
1-(1-Carboxy-2-phenylethylcarbamoyl)-3-(4-nonylbenzylamino)cyclopentane-carboxylic acid
3-{4-[5-(3-Chloro-4-ethoxyphenyl)pentyloxy]benzylamino}cyclopentane-1,1-dicarboxylic acid
3-[4-(4-Phenyl-5-trifluoromethylthiophen-2-ylmethoxy)benzylamino]cyclopentane-1,1-dicarboxylic acid enantiomer B
3-{Methyl-[4-(4-phenyl-5-trifluoromethylthiophen-2-ylmethoxy)benzyl]amino}cyclopentane-1,1-dicarboxylic acid
3-[(2-Fluoro-4′-heptylbiphenyl-4-ylmethyl)amino]cyclobutane-1,1-dicarboxylic acid
3-(4-Nonylbenzylamino)cyclobutane-1,1-dicarboxylic acid
3-(4-Decylbenzylamino)cyclobutane-1,1-dicarboxylic acid
3-[(4-Decylbenzyl)methylamino]cyclobutane-1,1-dicarboxylic acid
3-(4-Decyl-2,3-difluorobenzylamino)cyclobutane-1,1-dicarboxylic acid
3-[(2-Fluoro-4′-heptylbiphenyl-4-ylmethyl)amino]-1-methylcarbamoylcyclobutanecarboxylic acid—Isomer A
3-[(2-Fluoro-4′-heptylbiphenyl-4-ylmethyl)amino]-1-methylcarbamoylcyclobutanecarboxylic acid—Isomer B
3-[(4-Decylbenzyl)methylamino]cyclobutane-1,1-dicarboxylic acid ethyl ester
3-[(4-Decyl-2,3-difluorobenzyl)methylamino]cyclobutane-1,1-dicarboxylic acid
3-[(4-Decyl-2,3-difluorobenzyl)methylamino]cyclobutane-1,1-dicarboxylic acid ethyl ester
3-[4-(4-Phenyl-5-trifluoromethylthiophen-2-ylmethoxy)benzylamino]cyclobutane-1,1-dicarboxylic acid
1-(4-Decylbenzyl)piperidine-4,4-dicarboxylic acid
1-(4-Nonylbenzyl)piperidine-4,4-dicarboxylic acid
1-(4-Decyl-2,3-difluorobenzyl)piperidine-4,4-dicarboxylic acid
1-(4-Octylbenzyl)piperidine-4,4-dicarboxylic acid
1-(3-Chloro-4-nonyloxybenzyl)piperidine-4,4-dicarboxylic acid
1-(2-Fluoro-4′-heptylbiphenyl-4-ylmethyl)piperidine-4,4-dicarboxylic acid
1-(4-Decylbenzyl)piperidine-4,4-dicarboxylic acid ethyl ester
1-(3-Chloro-4-decyloxybenzyl)piperidine-4,4-dicarboxylic acid ethyl ester
1-(4-Decyl-2,3-difluorobenzyl)piperidine-4,4-dicarboxylic acid ethyl ester
1-(2-Fluoro-4′-heptylbiphenyl-4-ylmethyl)piperidine-4,4-dicarboxylic acid ethyl ester
1-(4-Decyl-2,3-difluorobenzyl)piperidine-4,4-dicarboxylic acid methyl ester
1-(4-Decyl-2,3-difluorobenzyl)piperidine-4,4-dicarboxylic acid isopropyl ester
1-(4-Decylbenzyl)-4-ethylcarbamoylpiperidine-4-carboxylic acid
1-(4-Decylbenzyl)-4-propylcarbamoylpiperidine-4-carboxylic acid
1-(2,3-difluoro-4-nonylbenzyl)-4-ethylcarbamoylpiperidine-4-carboxylic acid
4-[(4-Decyl-2,3-difluorobenzyl)methylamino]cyclohexane-1,1-dicarboxylic acid diethyl ester
4-[(2,3-Difluoro-4-nonylbenzyl)methylamino]cyclohexane-1,1-dicarboxylic acid diethyl ester
4-(2,3-Difluoro-4-nonylbenzylamino)cyclohexane-1,1-dicarboxylic acid
4-[(2,3-Difluoro-4-nonylbenzyl)methylamino]cyclohexane-1,1-dicarboxylic acid ethyl ester
4-[(4-Decyl-2,3-difluorobenzyl)methylamino]cyclohexane-1,1-dicarboxylic acid ethyl ester
4-(4-Decyl-2,3-difluorobenzylamino)cyclohexane-1,1-dicarboxylic acid
4-[(2,3-Difluoro-4-nonylbenzyl)methylamino]cyclohexane-1,1-dicarboxylic acid
4-[(4-Decyl-2,3-difluorobenzyl)methylamino]cyclohexane-1,1-dicarboxylic acid
1-[4-(4-Phenyl-5-trifluoromethylthiophen-2-ylmethoxy)benzyl]-piperidine-4,4-dicarboxylic acid
1-(4-Decylbenzyl)azetidine-3,3-dicarboxylic acid
2-[4-(3-Chloro-4-decyloxybenzylamino)but-2-ynyl]-2-methylmalonic acid
2-{4-[(2-Fluoro-4′-heptylbiphenyl-4-ylmethyl)amino]but-2-ynyl}-2-methylmalonic acid
2-{4-[Isopropyl(4-nonylbenzyl)amino]but-2-ynyl}-2-methylmalonic acid
2-{4-[Cyclopropyl(4-nonylbenzyl)amino]but-2-ynyl}-2-methylmalonic acid
2-{4-[(2-Fluoro-4′-hexylbiphenyl-4-ylmethyl)methylamino]but-2-ynyl}-2-methylmalonic acid
2-Methyl-2-{4-[methyl(4-nonylbenzyl)amino]but-2-ynyl}malonic acid
2-{4-[(2,3-Difluoro-4-nonylbenzyl)methylamino]but-2-ynyl}-2-methylmalonic acid.
2-{4-[Ethyl-(4-nonylbenzyl)amino]but-2-ynyl}-2-methylmalonic acid
2-{4-[(2,3-Difluoro-4-nonylbenzyl)amino]but-2-ynyl}-2-methylmalonic acid
2-[4-(4-Decyl-2,3-difluorobenzylamino)but-2-ynyl]-2-methylmalonic acid
2-{4-[(4-Decylbenzyl)methylamino]but-2-ynyl}-2-methylmalonic acid
2-{4-[(3-Chloro-4-nonyloxybenzyl)methylamino]but-2-ynyl}-2-methylmalonic acid.
2-Ethyl-2-{4-[(2-fluoro-4′-hexylbiphenyl-4-ylmethyl)amino]but-2-ynyl}malonic acid
2-Methyl-2-{4-[4-(4-phenyl-5-trifluoromethylthiophen-2-ylmethoxy)benzylamino]but-2-ynyl}malonic acid
as well as their enantiomers, diatereoisomers, geometrical isomers, mixtures thereof,
free forms and pharmaceutically acceptable salts, hydrates, solvates and esters.

18. Compound according to claim 1, selected from the group consisting in:
3-(4-nonylbenzylamino)cyclopentane-1,1-dicarboxylic acid
3-(4-decylbenzylamino)cyclopentane-1,1-dicarboxylic acid
3-(methyl-4-nonylbenzylamino)cyclopentane-1,1-dicarboxylic acid hydrochloride
3-(2,3-Difluoro-4-nonylbenzylamino)cyclopentane-1,1-dicarboxylic acid
3-[Methyl-(4-nonylbenzyl)amino]cyclopentane-1,1-dicarboxylic acid ethyl ester
3-(4-Decyl-2,3-difluorobenzylamino)cyclopentane-1,1-dicarboxylic acid
3-[(2,3-Difluoro-4-nonylbenzyl)methylamino]cyclopentane-1,1-dicarboxylic acid
3-[(4-Decyl-2,3-difluorobenzyl)methylamino]cyclopentane-1,1-dicarboxylic acid
3-[(4-Decyl-2,3-difluorobenzyl)methylamino]cyclopentane-1,1-dicarboxylic acid enantiomer B
3-[(4-Decyl-2,3-difluorobenzyl)methylamino]cyclopentane-1,1-dicarboxylic acid enantiomer B
3-[(4-Decyl-2,3-difluoro-benzyl)methylamino]-cyclopentane-1,1-dicarboxylic acid ethyl ester enantiomer B
3-[(4-Decyl-2,3-difluoro-benzyl)methylamino]-cyclopentane-1,1-dicarboxylic acid ethyl ester enantiomer A
3-[(4-Decyl-2,6-difluorobenzyl)methylamino]cyclopentane-1,1-dicarboxylic acid
3-[(4-Decyl-2,3-difluorobenzyl)methylamino]cyclobutane-1,1-dicarboxylic acid
3-[(4-Decyl-2,3-difluorobenzyl)methylamino]cyclobutane-1,1-dicarboxylic acid ethyl ester
1-(4-Decyl-2,3-difluorobenzyl)piperidine-4,4-dicarboxylic acid
1-(2-Fluoro-4′-heptylbiphenyl-4-ylmethyl)piperidine-4,4-dicarboxylic acid
1-(4-Decyl-2,3-difluorobenzyl)piperidine-4,4-dicarboxylic acid ethyl ester
1-(4-Decyl-2,3-difluorobenzyl)piperidine-4,4-dicarboxylic acid methyl ester
1-(4-Decyl-2,3-difluorobenzyl)piperidine-4,4-dicarboxylic acid isopropyl ester
1-(2,3-difluoro-4-nonylbenzyl)-4-ethylcarbamoyl piperidine-4-carboxylic acid
as well as their enantiomers, diastereoisomers, geometrical isomers, mixtures thereof, free forms and pharmaceutically acceptable salts, hydrates, solvates and esters.

19. Process of preparation of a compound according to claim 1, further comprising the step of reacting a compound of formula (III): 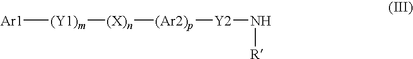
with a compound of formula (IV):
O═(Y3′)_{q}-Z(CO₂R″)(COR′″) (IV)
wherein Ar1, Y1, X, Ar2, Y2, R′, Z, R″, R′″, m, n, p, q are defined as in formula (I), provided that when q=0, the keto function is attached to a carbon atom of Z, and when q=1, Y3′ is such that —CH2-Y3′- corresponds to Y3 as defined in formula (I).

20. Process according to claim 19, wherein said reaction is carried out under reductive amination conditions.

21. Process of preparation of a compound according to claim 1, comprising the step of reacting a compound of formula (V):
Ar1(Y1)ₘ-(X)ₙ-(Ar2)ₚ-Y2-SO2Alkyl (V)
with a compound of formula (IX): 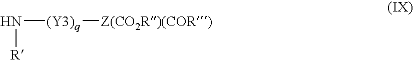
wherein Ar1, Y1, X, Ar2, Y2, R′, Y3, Z, R″, R′″, m, n, p, q are defined as in formula (I).

22. Process according to claim 21, wherein said reaction is carried out under nucleophilic displacement conditions.

23. Process of preparation of a compound according to claim 1, comprising the step of reacting a compound of formula (VII):
Ar1-(Y1)ₘ-(X)ₙ-(Ar2)ₚ—Y2=O (VII)
with a compound of formula (X):
H2N(Y3′)_{q}-Z-(CO2R″)(COR′″) (X)
wherein Ar1, Y1, X, Ar2, Y2, Z, R″, R′″, m, n, p, q are defined as in formula (I), Y3′ is such that —CH2-Y3′- corresponds to Y3 as defined in formula (I).

24. Process according to claim 23, wherein said reaction is carried out under reductive conditions.

25. Process according to claim 19 to 22 claim 19, wherein said coupling reaction is followed by further modifying Ar1, Y1, X, Ar2, Y2, R′, Z, R″, R′″ of the compound of formula (I) obtained so as to obtain the desired compound of formula (I).

26. Process according to claim 19, which further comprises the additional step of isolating the compound of formula (I).

27. A pharmaceutical composition comprising a compound of formula (I) according to claim 1 with a pharmaceutically acceptable excipient or carrier.

28. Use of a compound of formula (I) 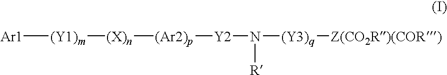
wherein
Ar1 represents an Aryl or Heteroaryl group optionally substituted by 1 up to 5 R group(s),
wherein, each R, identical or different, is selected from the group consisting in Halogen atom, perhalogenoalkyl, -Alkyl, —OAlkyl, —OH, —COOR7, —CONR7R8, -Alkyl-Hal, —OAlkyl-Hal, NO2, —CN, —NR7R8, -AlkylAryl, -Aryl, —S(O)lR7, -Alkenyl, —Si(Alkyl);
wherein R7 and R8, identical or different, are chosen from the group consisting in H Alkyl; Cycloalkyl; Aryl; -AlkylAryl; Heteroaryl; wherein Alkyl, Cycloalkyl, and/or Aryl is(are) optionally substituted by one or more identical or different Halogen atom, polyfluoroalkyl, Aryl, —COOR7
or R7 and R8 form together with the N atom to which they are attached a Heterocycle;
l is 0, 1 or 2;
Y1 represents an -Alkyl-chain, optionally substituted by one or more R as defined above, and optionally comprising one or more unsaturation(s) and/or heteroatom(s) and/or a residue chosen from the group consisting in -Alkenyl-, -Alkynyl-, —CH═N—, —N═CH—, —CH═N—O—, —O—N═CH—, —C(═O)—, —C(═O)(OR7)-, —C(═O)(NR7)-, —N═N—, —S(O)l-;
m is 0 or 1;
X represents a heteroatom;
n is 0 or 1;
—Ar2- represents an -Aryl-group optionally substituted by one or more R group as defined above or wherein 2 R may form together with the atoms to which they are attached a fused cyclic, aryl or heteroaryl ring;
p is 0 or 1;
—Y2- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7), —N═N—, —S(O)l-;
—R′ represents a hydrogen atom, or a -Cycloalkyl or an -Alkyl chain, the cycloalkyl or alkyl being optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7)-, —N═N—, —S(O)l-, such as -Alkenyl-Alkyl, -Alkynyl-Alkyl, —CH═N-Alkyl, —N═CH-Alkyl, —CH═N—O-Alkyl, —O—N═CH-Alkyl, —C(═O)-Alkyl, —C(═O)O-Alkyl, —C(═O)(NR7)-Alkyl, —N═N-Alkyl, —S(O)l-Alkyl;
—Y3- represents an -Alkyl-chain, optionally substituted by one or more R as defined above, and optionally comprising one or more unsaturation(s) or heteroatom(s) and/or a residue chosen from the group consisting in -Alkenyl-, -Alkynyl-, —CH═N—, —N═CH—, —CH═N—O—, —O—N═CH—, —C(═O)—, —C(═O)O—, —C(═O)(NR7)-, —N═N—, —S(O)l-;
q is 0 or 1;
Z represents a -Cycloalkyl< group or an -Heterocyclic<, -Aryl<, -Heteroaryl< group or a —CH<, —C(alkyl)< group, or R′ and Z form together with the N atom to which they are attached a Heterocyclic or Heteroaryl group;
wherein (CO2R″) and (COR′″) can be attached to the same atom or two adjacent atoms;
—R″ represents a hydrogen atom or an Alkyl chain, optionally substituted by one or more R as defined above, and optionally comprising one or more unsaturation(s);
—R′″ represents —OH, —OAlkyl, H, —NR7OR8, —NR7R8, natural or synthetic amino acids, wherein R7 and R8 are defined as above;
or R″ and R′″ form together a ring with the atom(s) to which they are attached to form a cyclic intramolecular bis carbonyl group, including Meldrum acid derivatives;
as well as their enantiomers, diatereoisomers, geometrical isomers, mixtures thereof, free forms and pharmaceutically acceptable salts, hydrates, solvates and esters for the preparation of a medicament acting as an agonist at human S1P1 receptors selectively, for administration to a patient in the need thereof.

29. Use according to claim 28, wherein said compound is defined as in claim 1.

30. Use according to claim 28, wherein said compound interacts with sphingosine phosphate receptor.

31. Use according to claim 28, wherein said compound acts as an agonist at sphingosine phosphate receptor.

32. Use according to claim 28, wherein said compound is suitable for decreasing circulating lymphocytes in blood in a patient in the need thereof.

33. Use according to claim 28, wherein said compound is for treating and/or preventing transplant rejection, immune disorders, autoimmune disorders, inflammatory and chronic inflammatory conditions that include rheumatoid arthritis, asthma, pollinosis, psoriasis, myocarditis, atopic dermatitis, lymphocytic leukemias, lymphomas, multiple sclerosis, lupus erythematosus, inflammatory bowel diseases, diabetes mellitus, glomerulo-nephritis, atherosclerosis, multiorgan failure, sepsis, pneumonia as well as disorders related to impaired vascular integrity, deregulated angiogenesis.

34. Use according to claim 28, wherein said medicament is for treating and/or preventing tissue graft rejection.
